(19) United States
(12) Patent Application Publication
(10) Pub. No.: US 2018/0042765 A1
(43) Pub. Date: Feb. 15, 2018

(54) METHODS AND DEVICES FOR TREATING POSTERIOR OCULAR DISORDERS

(71) Applicant: CLEARSIDE BIOMEDICAL, INC., Alpharetta, GA (US)

(72) Inventors: Glenn NORONHA, Alpharetta, GA (US); Christopher John BROOKS, Glen Cove, NY (US); Rafael Victor ANDINO, Grayson, GA (US); Samirkumar PATEL, Alpharetta, GA (US); Daniel WHITE, Alpharetta, GA (US)

(21) Appl. No.: 15/319,045

(22) PCT Filed: Jun. 17, 2015
(86) PCT No.: PCT/US15/36299

§ 371 Date: Dec. 15, 2016

Related U.S. Application Data
(60) Provisional application No. 62/013,209, filed on Jun. 17, 2014.
(60) Provisional application No. 62/018,148, filed on Jun. 27, 2014.
(60) Provisional application No. 62/063,792, filed on Oct. 14, 2014.
(60) Provisional application No. 62/155,367, filed on Apr. 30, 2015.
(60) Provisional application No. 62/156,802, filed on May 4, 2015.

(51) Int. Cl. A61F 9/00

(52) U.S. Cl. CPC A61F 9/0008; A61K 9/0048

(57) ABSTRACT

The present invention relates to a methods and devices for treating uveitis, macular edema associated with uveitis and macular edema associated with retinal vein occlusion in a human subject in need thereof. In certain aspects, devices provided herein include a medicament container defining a lumen configured to contain a medicament, a distal end portion of the medicament container including a coupling portion configured to be removably coupled to a needle assembly, a proximal end portion of the medicament container including a flange and a longitudinal shoulder; a piston assembly including a distal end portion movably disposed within the lumen of the medicament container; and a handle coupled to a proximal end portion of the piston assembly.

### CROSS-REFERENCE TO RELATED APPLICATIONS

This application claims priority to and the benefit of U.S. Provisional Patent Application Ser. No. 62/156,802 entitled, “Methods and Devices for Treating Posterior Ocular Disorders,” filed May 4, 2015; U.S. Provisional Patent Application Ser. No. 62/063,792 entitled, “Apparatus and Methods for Ocular Injection,” filed Oct. 14, 2014; U.S. Provisional Patent Application Ser. No. 62/018,148 entitled, “Methods and Devices for Treating Posterior Ocular Disorders,” filed Jun. 27, 2014; and U.S. Provisional Patent Application Ser. No. 62/013,209 entitled, “Methods and Devices for the Treatment of Macular Edema Associated with Uveitis,” filed Jun. 17, 2014, the disclosures of which are incorporated herein by reference in their entireties.

This application also claims priority to and the benefit of U.S. Provisional Patent Application Ser. No. 62/155,367 entitled, “Methods and Devices for Treating Posterior Ocular Disorders,” filed Apr. 30, 2015, the disclosure of which is incorporated herein by reference in its entirety.

### BACKGROUND OF THE INVENTION

Uveitis is the most common form of inflammation of the choroid and surrounding tissues in the eye, and one of the most frequent causes of blindness in the developed world. Uveitis, which can affect both eyes and is often initially diagnosed in individuals 20 to 50 years, currently accounts for 10% of vision loss/blindness in the United States and 15% worldwide, mainly occurring in the 20-50 year age group. According to studies measuring incidence and prevalence of uveitis, more than 160,000 people are diagnosed with uveitis in the United States each year. Uveitis can be infectious, meaning it is caused by an immune response to fight an infection inside the eye, or non-infectious. Non-infectious uveitis accounts for approximately 80% of all uveitis cases.

Prolonged or severe inflammation in the back of the eye can result in the breakdown of cells at the interface of the retina and choroid, resulting in the leakage and accumulation of fluid in the macular region of the retina. This build-up of fluid can cause abnormal swelling of the macula, or macular edema, which can rapidly result in distortion of vision and eventually blindness. Because of the macula's critical role in central vision, macular edema can rapidly result in distortion of vision and eventually blindness. Macular edema is the most frequent cause of visual impairment among patients with uveitis. Because uveitis can become chronic or recurrent if not adequately treated, some patients may become refractory, or unresponsive, to treatment, leading to irreversible blindness. Further, macular edema may persist even with successful control of the inflammatory response.

Corticosteroids are currently regarded as the most effective treatment for non-infectious uveitis. Although used to treat some uveitis patients, corticosteroid eye drops are generally ineffective in treating patients with macular edema associated with uveitis because they cannot reach the choroid and retina in effective concentrations. Oral or other systemically administered corticosteroids and immunosuppressive agents can be effective in treating patients with macular edema associated with uveitis, but their long-term use is associated with harmful side effects.

RVO is a condition that affects vision, resulting from a blockage in one of the veins returning blood flow from the retina. RVO is the second most common cause of vision loss due to retinal vascular disease. RVO affects 16.4 million adults worldwide, according to a 2010 study published in the journal Ophthalmology (Rogers et al. (2010). Ophthalmology 117, pp. 313-319)). Inadequately treated macular edema associated with RVO can cause significant loss in visual acuity and eventually lead to blindness.

The present invention provides novel methodology and devices for the treatment of macular edema associated with uveitis, macular edema following retinal vein occlusion (RVO), thereby addressing key needs in the field of ocular therapeutics.

### SUMMARY OF THE INVENTION

This invention is generally related to ophthalmic therapies, and more particularly to methods and devices that allow for infusion of a fluid drug formulation into ocular tissues for targeted, localized treatment, for example, for the treatment of macular edema associated with uveitis or macular edema following retinal vein occlusion.

In one aspect, the present invention relates to a method of treating macular edema associated with uveitis in a human subject in need thereof. The method includes non-surgically administering an effective amount of a drug formulation to the suprachoroidal space (SCS) of the eye of the human subject in need of treatment for at least one dosing session. In another embodiment, the method is carried out over multiple dosing sessions, for example, dosing sessions that are spaced apart by from about 14 days, about 30 days (e.g., monthly), about 60 days (e.g., every other month), about 90 days (e.g., every third month or every 12 weeks) or about every 180 days.

In one embodiment, the drug formulation comprises an anti-inflammatory drug. In a further embodiment, the drug is a steroid. In even a further embodiment, the steroid is triamcinolone. The uveitis, in one embodiment is non-infectious uveitis or infectious uveitis. The uveitis (infectious or non-infectious), in one embodiment, is intermediate, posterior or pan uveitis.

In another aspect, the present invention relates to a method of treating macular edema associated with retinal vein occlusion (RVO) in a human subject in need thereof. The method includes non-surgically administering an effective amount of a drug formulation to the suprachoroidal space (SCS) of the eye of the human subject in need of treatment for at least one dosing session. In one embodiment, the drug formulation comprises an anti-inflammatory drug. In a further embodiment, the drug is a steroid, e.g., triamcinolone. In another embodiment, SCS administration of an anti-inflammatory drug formulation is combined with intravitreal injection of a VEGF modulator to treat a patient in need of macular edema associated with RVO treatment. In a further embodiment, the SCS and intravitreal injections are carried out in the same at least one dosing session.

In one embodiment of the method for treating macular edema associated with uveitis and/or the method for treating macular edema associated with retinal vein occlusion (RVO), subsequent to at least one dosing session, e.g., from about 1 week to about 14 weeks after at least one dosing dession, e.g., about 12 weeks after a dosing session, the patient experiences an improvement in visual acuity as measured by best corrected visual acuity of ≧10 letters, ≧15 letters or ≧25 letters, as compared to patient's visual acuity prior to the at least one dosing session. In one embodiment of the method for treating macular edema associated with uveitis, subsequent to at least one dosing session, e.g., from about 1 week to about 14 weeks after at least one dosing dession, e.g., about 4 weeks, about 8 weeks or about 12 weeks after at least one dosing session, the patient experiences a decrease in retinal thickness (e.g., central subfield thickness) as compared to the patient's retinal thickness prior to the at least one dosing session. In one embodiment, the decrease in retinal thickness is ≧25 μm, ≧50 μm, ≧75 μm or ≧100 μm. In some embodiments, the methods set forth herein are carried out by inserting a distal end portion of a needle of a medical injector into a target tissue to define a delivery passageway within the target tissue and such that a distal end surface of a hub of the medical injector is in contact with a target surface of the target tissue. A force is exerted (e.g., a manual force by a user) on an actuator of the medical injector when the distal end surface of the hub is in contact with the target surface. The medical injector is configured such that the force is sufficient to move a distal end portion of the actuator within the medicament container when the distal end portion of the needle is disposed within a first region of the target tissue. The medical injector is configured such that the force is insufficient to move the distal end portion of the actuator within the medicament container when the distal end portion of the needle is disposed within a second region of the target tissue. In some embodiments, the force has a magnitude of less than about 6 N. A substance, e.g., a drug formulation, in response to the exertion, is conveyed from the medicament container into the target tissue via the needle when the distal end portion of the needle is disposed within the first region of the target tissue. The first region can be, for example, a suprachoroidal space of the eye, a lower portion of the sclera and/or an upper portion of the choroid. In some embodiments, the first region can be a retina of the eye.

In some embodiments of the methods provided herein, a distal end portion of a needle of a medical injector is inserted into a target tissue to define a delivery passageway within the target tissue. The insertion is performed such that a centerline of the needle and a surface line tangent to a target surface of the target tissue define an angle of entry of between about 75 degrees and about 105 degrees. A distal end surface of a hub of the medical injector is placed into contact with a target surface of the target tissue to fluidically isolate the delivery passageway. After the distal end surface of the hub is placed into contact with the target surface, a substance, e.g., drug formulation, is conveyed into the target tissue via the needle.

In some embodiments, a distal end portion of a needle of a medical injector is inserted into an eye to define a delivery passageway within a sclera of the eye. After the distal end portion of the needle is inserted into the eye, a force (e.g., a manual force by a user) is applied to the medical injector when a distal tip of the needle is disposed within at least one of a suprachoroidal space or a lower portion of the sclera, the force being insufficient to convey the substance from the medicament container via the needle when the distal tip of the needle is disposed within an upper portion of the sclera of the eye.

### BRIEF DESCRIPTION OF THE DRAWINGS

FIG. 1 is a cross-sectional view of an illustration of the human eye.

FIG. 2 is a cross-sectional view of a portion of the human eye of FIG. 1 taken along the line 2-2.

FIGS. 3 and 4 are cross-sectional views of a portion of the human eye of FIG. 1 taken along the line 3-3, illustrating the suprachoroidal space without and with, respectively, the presence of a fluid.

FIG. 5 is a perspective view of a medical injector according to an embodiment.

FIG. 6 is a partially exploded view of the medical injector of FIG. 5.

FIG. 7 is an exploded view of the medical injector of FIG. 5, shown without a needle cap.

FIG. 8 is a front view of a handle included in the medical injector of FIG. 5.

FIG. 9 is a cross-sectional view of the handle of FIG. 8 taken along the line 9-9.

FIG. 10 is a perspective view of a barrel included in the medical injector of FIG. 5.

FIG. 11 is an exploded view of a needle hub included in the medical injector of FIG. 5.

FIG. 12 is a front view of the needle hub of FIG. 9.

FIG. 13 is an enlarged view of a portion of the needle hub of FIG. 12, identified by the region Z₁.

FIG. 14 is a rear perspective view of a needle cap included in the medical injector of FIG. 5.

FIG. 15 is a front view of the medical injector of FIG. 5.

FIG. 16 is a cross-sectional view of the medical injector of FIG. 5, taken along the like 16-16 in FIG. 15.

FIG. 17 is a view of the medical injector of FIG. 5 in use during an injection procedure into the human eye.

FIG. 18 is an enlarged view of a portion of the medical injector of FIG. 5 and the human eye, identified in FIG. 17 by the region Z₂.

FIG. 19 is an exploded view of a needle hub configured for use with the medical injector of FIG. 5, according to an embodiment.

FIG. 20 is a front view of the needle hub of FIG. 19.

FIG. 21 is a flowchart illustrating a method of using a medical injector to inject a medicament into an eye.

FIG. 22 is a graph of mean change in intraocular pressure vs. hours or weeks post-treatment.

FIG. 23 is a graph of improvement in best corrected visual acuity (mean change in visual acuity score (letters read) from baseline (base logMAR) vs. weeks post-treatment. 0.1 logMAR=1 line=5 letters.

FIG. 24 is a plot of mean reduction in retinal thickness vs. weeks post-treatment.

FIG. 25 are optical coherence tomography images of the eyes of a bilateral chronic uveitis patient with macular edema, prior to (top images) and subsequent to (bottom images) SCS TA injection (left 2 images) or sub-tenon TA injection (right 2 images).

FIG. 26 are optical coherence tomography images of the eyes of a bilateral chronic uveitis patient with macular edema, prior to (top images) and subsequent to (bottom images) SCS TA injection (right 2 images, right eye) or Ozurdex (dexamethasone 0.7 mg intravitreal implant) (left 2 images, left eye).

FIG. 27 illustrates distribution in various parts of the eye following intravitreal and SCS injection of Triesence in rabbits.

FIG. 28A-F illustrate distribution of TA in various parts of the eye (28A: sclera-choroid-outer retina; FIG. 28B: inner retina; FIG. 28C: vitreous; FIG. 28D: aqueous humour; FIG. 28E: lens; FIG. 28F: iris-ciliary body) following intravitreal and SCS injection of Triesence.

FIGS. 29 and 30 illustrate TA concentration for Trisence and CLS-TA over a 90-day period in either the sclera-choroid-outer retina (FIG. 29) or the inner retina (FIG. 30).

FIG. 31 is a bar graph showing the cumulative ophthalmoscopy inflammation scores (mean±SD) for each treatment group. Group 1: Negative control (LPS/BSS SCS); Group 2: Oral high dose prednisone (LPS/Prednisonel mg/kg/day PO); c: Group 2 mean cumulative inflammation score on Day 3 was significantly lower than Group 1 (p<0.034); Group 3: CLS-TA (LPS/2 mg CLS-TA) a: Group 3 mean cumulative inflammation score on Day 1 was significantly lower than Group 1 (p=0.04); b: Group 3 mean cumulative inflammation score on Day 2 was significantly lower than Group 1 (p=0.023); d: Group 3 mean cumulative inflammation score on Day 3 was significantly lower than Group 1 (p<0.034); Group 4: Oral low dose prednisone (LPS/Prednisone 0.1 mg/kg/day PO).

FIG. 32 is a bar graph showing the intraocular pressure (mmHg; mean±SD) for each treatment group over the study period. Group 1: Negative control (LPS/BSS SCS); Group 2: Oral high dose prednisone (LPS/Prednisone 1 mg/kg/day PO); Group 3: CLS-TA (LPS/2 mg CLS-TA); Group 4: Oral low dose prednisone (LPS/Prednisone 0.1 mg/kg/day PO); Group 4 mean IOP on Day 3 was significantly lower than Groups 1, 2, and 3 (P<0.0065).

FIG. 33 are graphs showing the mean histologic score for various treatment groups for the anterior segment (left) and posterior segment (right).

### DETAILED DESCRIPTION OF THE INVENTION

Methods, devices and drug formulations are provided herein for treating posterior ocular disorders, for example macular edema associated with uveitis (e.g., infectious or non-infectious uveitis) and macular edema associated with retinal vein occlusion (RVO) in a human subject in need thereof. In one embodiment, the RVO is branch retinal vein occlusion (BRVO), hemiretinal vein occlusion (HRVO) or central retinal vein occlusion (CRVO). In one embodiment, the uveitis is intermediate, posterior or pan uveitis, and can be infectious or non-infectious uveitis.

Uveitis is one of the most frequent causes of blindness in the developed world. Based on prevalence data published in the journal Ophthalmology in 2004 and United States census data for 2010, it is estimated that approximately 350,000 individuals in the United States suffer from some form of uveitis. Uveitis can be either infectious or non-infectious. Non-infectious uveitis accounts for approximately 80% of all uveitis cases. Macular edema associated with uveitis is the predominant cause of blindness or visual impairment among patients with uveitis, accounting for approximately 30% of cases of blindness in uveitis patients. Because uveitis can become chronic or recurrent if not adequately treated, some patients may become refractory, or unresponsive, to treatment, leading to irreversible blindness. Currently, there are no FDA approved treatments specifically indicated for macular edema associated with non-infectious uveitis.

Intravitreal injections result in drugs diffusing throughout the eye, including into the lens, iris and ciliary body at the front of the eye, which for some drugs, has been associated with safety issues, such as cataracts and elevated intraocular pressure (TOP) levels. Specifically, intravitreal administration of triamcinolone (TA) has been associated with cataracts and increases in IOP levels in 20% to 60% of patients. Because SCS injection of drugs appears to result in drug remaining localized in the retina and choroid without substantial diffusion to the vitreous or the front portion of the eye, without wishing to be bound by theory, it is thought that SCS injection has the potential to reduce the incidence of these side effects.

Current treatments for retinal vein occlusion (RVO) require monthly intravitreal injections of anti-VEGF drugs. For patients experiencing macular edema associated with RVO, without wishing to be bound by theory, it is thought that a combination of a standard intravitreal injection of an anti-VEGF drug that addresses the vascular aspect of the disease, together with an SCS injection of an anti-inflammatory compound (such as a steroid, e.g., triamcinolone), which addresses the inflammatory aspect of RVO, may have similar or better efficacy with a reduction in the frequency of required anti-VEGF treatments from 30 days to 90 days. Accordingly, the dual strategy to useful for optimization of treatment of a patient.

The methods and devices provided herein, for example, for the treatment of macular edema associated with uveitis, macular edema associated with RVO, wet AMD and/or diabetic macular edema (DME), in one embodiment, are used to restore or improve visual function primarily by reducing the macular edema affecting the retina, the tissue that lines the inside of the eye and is the part of the eye primarily responsible for vision, and the choroid, the layer adjacent to the retina that supplies the retina with blood, oxygen and nourishment. Macular edema is the build-up of fluid that can cause abnormal swelling of the macula, the portion of the retina responsible for central vision and color perception. This swelling can rapidly result in deterioration of vision and can eventually lead to blindness.

As used herein, “non-surgical” ocular drug delivery devices and methods refer to methods and devices for drug delivery that do not require general anesthesia and/or retrobulbar anesthesia (also referred to as a retrobulbar block). Alternatively or additionally, a “non-surgical” ocular drug delivery method is performed with an instrument having a diameter of 28 gauge or smaller. Alternatively or additionally, “non-surgical” ocular drug delivery methods do not require a guidance mechanism that is typically required for ocular drug delivery via a shunt or cannula.

The non-surgical posterior ocular disorder treatment methods and devices described herein are particularly useful for the local delivery of drugs to the posterior region of the eye, for example the retinochoroidal tissue, macula, retinal pigment epithelium (RPE) and optic nerve in the posterior segment of the eye. In another embodiment, the non-surgical methods and microneedles provided herein can be used to target drug delivery to specific posterior ocular tissues or regions within the eye or in neighboring tissue. In one embodiment, the methods described herein deliver drug specifically to the sclera, the choroid, the Brach's membrane, the retinal pigment epithelium, the subretinal space, the retina, the macula, the optic disk, the optic nerve, the ciliary body, the trabecular meshwork, the aqueous humor, the vitreous humor, and/or other ocular tissue or neighboring tissue in the eye of a human subject in need of treatment. The methods and microneedles provided herein, in one embodiment, can be used to target drug delivery to specific posterior ocular tissues or regions within the eye or in neighboring tissue.

In one embodiment of the methods described herein, a patient in need of treatment of macular edema associated with uveitis (e.g., infectious, non-infectious, intermediate, posterior or pan uveitis), macular edema associated with RVO (e.g., branch retinal vein occlusion (BRVO), hemiretinal vein occlusion (HRVO), or central retinal vein occlusion (CRVO)) is non-surgically administered a drug, e.g., an anti-inflammatory drug (e.g., triamcinolone) or a vascular endothelial growth factor (VEGF) modulator (e.g., VEGF antagonist) to the suprachoroidal space of one or both eyes for at least one dosing session. Non-surgical administration, in one embodiment, is achieved by inserting a microneedle into one or both eyes of the patient, for example the sclera, and injecting or infusing a drug formulation through the inserted microneedle and into the suprachoroidal space of the eye. In one embodiment, the effective amount of the drug administered to the SCS provides higher thereapeutic efficacy of the drug, compared to the therapeutic efficacy of the drug when the identical dosage is administered intravitreally, topically, intracamerally, parenterally or orally. In one embodiment, the microneedle drug delivery methods described herein precisely deliver the drug into the SCS for subsequent local delivery to nearby posterior ocular tissues (e.g., the retina and choroid) in need of treatment. The drug may be released into the ocular tissues from the infused volume (or, e.g., from microparticles or nanoparticles in the drug formulation) for an extended period, e.g., several hours or days or weeks or months, after the non-surgical drug administration has been completed. This beneficially can provide increased bioavailability of the drug relative, for example, to delivery by topical application of the drug formulation to ocular tissue surfaces, or increased bioavailability compared to oral, parenteral on intravitreal administration of the same drug dosage.

With the methods and microneedle devices described herein, the SCS drug delivery methods advantageously include precise control of the depth of insertion into the ocular tissue, so that the microneedle tip can be placed into the eye so that the drug formulation flows into the suprachoroidal space and into one or more posterior ocular tissues surrounding the SCS, e.g., the choroid and retina. In one embodiment, insertion of the microneedle is in the sclera of the eye. In one embodiment, drug flow into the SCS is accomplished without contacting underlying tissues with the microneedle, such as choroid and retina tissues.

The methods provided herein, in one embodiment, achieve delivery of drug to the suprachoroidal space, thereby allowing drug access to posterior ocular tissues (e.g., the choroid and retina) not obtainable via topical, parenteral, intracameral or intravitreal drug delivery. Because the methods provided herein deliver drug to the posterior ocular tissue for the treatment of a posterior ocular disorder, the suprachoroidal drug dose sufficient to achieve a therapeutic response and/or the frequency of dosing in a human subject treated with the methods provided herein is less than the intravitreal, topical, parenteral or oral drug dose or dosing schedule sufficient to elicit the same or substantially the same therapeutic response. In one embodiment, the SCS delivery methods described herein allow for decreased drug dose of the posterior ocular disorder treating drug, compared to the intravitreal, topical, intracameral parenteral or oral drug dose sufficient to elicit the same or substantially the same therapeutic response. In a further embodiment, the suprachoroidal drug dose sufficient to elicit a therapeutic response is 75% or less, or 50% or less, or 25% or less than the intravitreal, topical parenteral or oral drug dose sufficient to elicit a therapeutic response. The therapeutic response, in one embodiment, is a reduction in severity of a symptom/clinical manifestation of the posterior ocular disorder (macular edema associated with uveitis, macular edema associated with RVO, wet AMD)for which the patient is undergoing treatment, or a reduction in number of symptom(s)/clinical manifestation(s) of the posterior ocular disorder for which the patient is undergoing treatment.

The term “suprachoroidal space,” is used interchangeably with suprachoroidal, SCS, suprachoroid and suprachoroidia, and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the suprachoroidal space and the adjacent tissues. Those skilled in the art will appreciate that the suprachoroidal space frequently is expanded by fluid buildup because of some disease state in the eye or as a result of some trauma or surgical intervention. In the present description, however, the fluid buildup is intentionally created by infusion of a drug formulation into the suprachoroid to create the suprachoroidal space (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

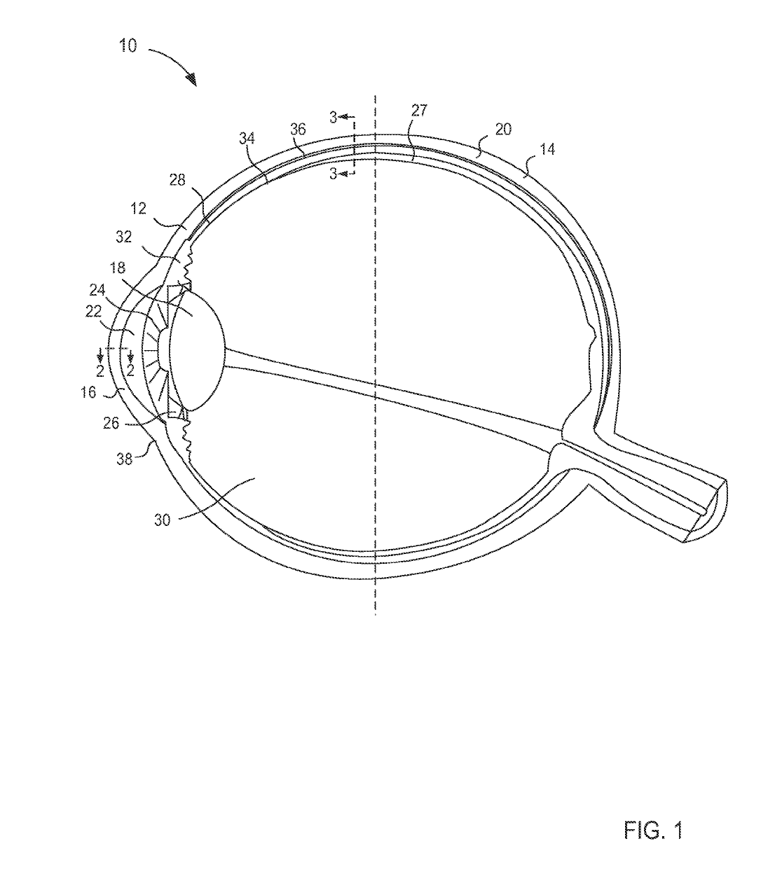

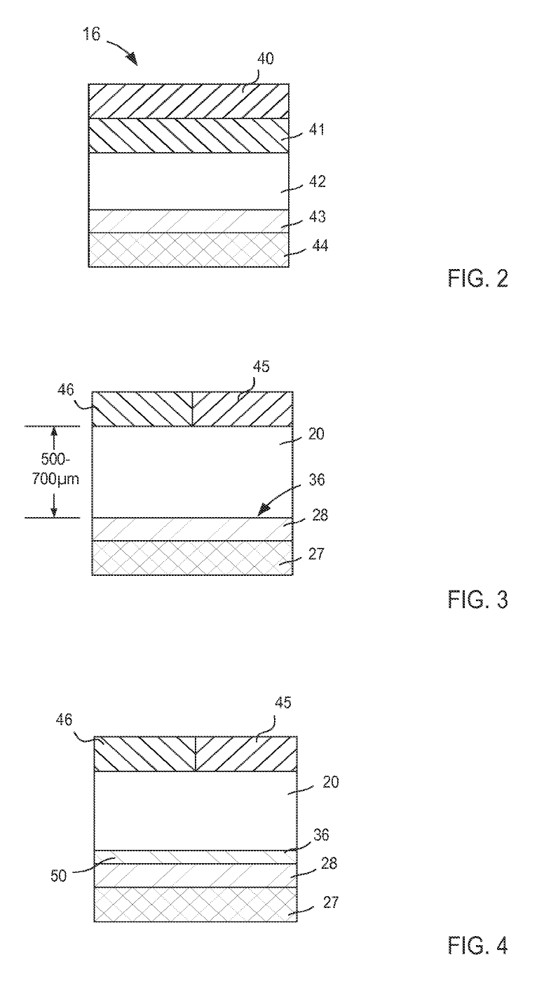

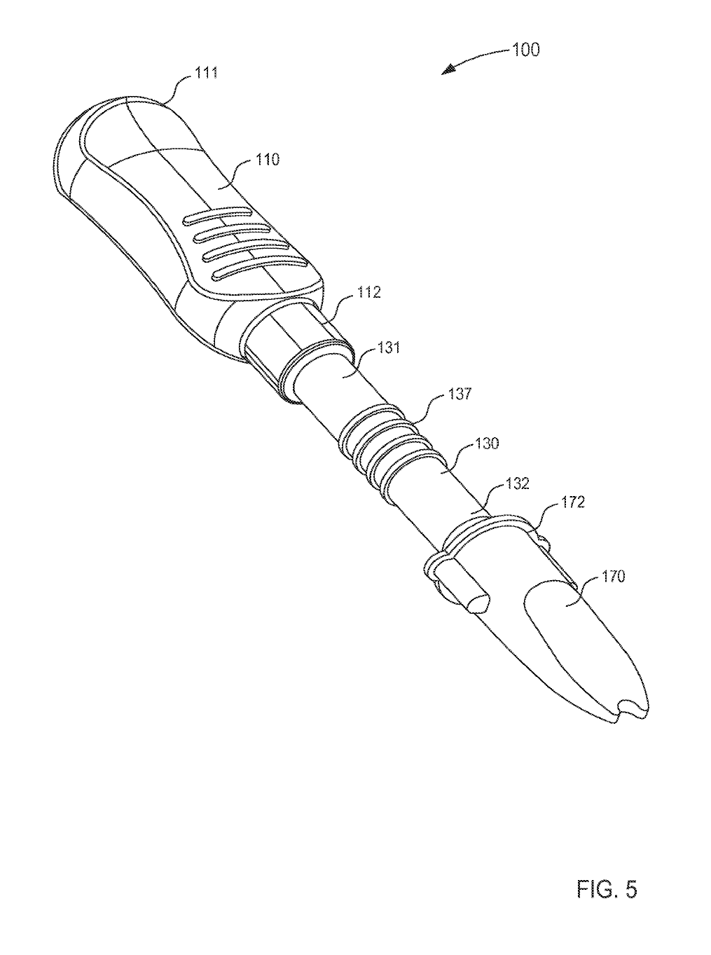

As used herein, “ocular tissue” and “eye” include both the anterior segment of the eye (i.e., the portion of the eye in front of the lens) and the posterior segment of the eye (i.e., the portion of the eye behind the lens). For reference, FIGS. 1-4 are a various views of a human eye 10 (with FIGS. 2-4 being cross-sectional views). While specific regions are identified, those skilled in the art will recognize that the proceeding identified regions do not constitute the entirety of the eye 10, rather the identified regions are presented as a simplified example suitable for the discussion of the embodiments herein. The eye 10 includes both an anterior segment 12 (the portion of the eye in front of and including the lens) and a posterior segment 14 (the portion of the eye behind the lens). The anterior segment 12 is bounded by the cornea 16 and the lens 18, while the posterior segment 14 is bounded by the sclera 20 and the lens 18. The anterior segment 12 is further subdivided into the anterior chamber 22, between the iris 24 and the cornea 16, and the posterior chamber 26, between the lens 18 and the iris 24. The cornea 16 and the sclera 20 collectively form a limbus 38 at the point at which they meet. The exposed portion of the sclera 20 on the anterior segment 12 of the eye is protected by a clear membrane referred to as the conjunctiva 45 (see e.g., FIGS. 2 and 3). Underlying the sclera 20 is the choroid 28 and the retina 27, collectively referred to as retinachoroidal tissue. A vitreous humour 30 (also referred to as the “vitreous”) is disposed between a ciliary body 32 (including a ciliary muscle and a ciliary process) and the retina 27. The anterior portion of the retina 27 forms an ora serrata 34. The loose connective tissue, or potential space, between the choroid 28 and the sclera 20 is referred to as the suprachoroid. FIG. 2 illustrates the cornea 16, which is composed of the epithelium 40, the Bowman's layer 41, the stroma 42, the Descemet's membrane 43, and the endothelium 44. FIG. 3 illustrates the sclera 20 with surrounding Tenon's Capsule 46 or conjunctiva 45, suprachoroidal space 36, choroid 28, and retina 27, substantially without fluid and/or tissue separation in the suprachoroidal space 36 (i.e., the in this configuration, the space is “potential” suprachoroidal space). As shown in FIG. 3, the sclera 20 has a thickness between about 500 nm and 700 nm. FIG. 4 illustrates the sclera 20 with the surrounding Tenon's Capsule 46 or the conjunctiva 45, suprachoroidal space 36, choroid 28, and retina 27, with fluid 50 in the suprachoroidal space 36.

The dashed line in FIG. 1 represents the equator of the eye 10. In some embodiments, the insertion site of any of the microneedles and/or methods described herein is between the equator and the limbus 38 (i.e., in the anterior portion 12 of the eye 10) For example, in some embodiments, the insertion site is between about two millimeters and 10 millimeters (mm) posterior to the limbus 38. In other embodiments, the insertion site of the microneedle is at about the equator of the eye 10. In still other embodiments, the insertion site is posterior the equator of the eye 10. In this manner, a drug formulation can be introduced (e.g., via the microneedle) into the suprachoroidal space 36 at the site of the insertion and can flow through the suprachoroidal space 36 away from the site of insertion during an infusion event (e.g., during injection).

The microneedle may extend from the base of the microneedle device at any angle suitable for insertion into the eye 10. In a particular embodiment, the microneedle extends from the base at an angle of about 90 degrees to provide approximately perpendicular insertion of the microneedle into the surface of the eye. In another embodiment, the microneedle extends from the base at an angle from about 60 to about 110 degrees, from about 70 degrees to about 100 degrees, from about 80 degrees to about 90 degrees, or from about 85 degrees to about 95 degrees.

The microneedle device may comprise a means for controllably inserting, and optionally retracting, the microneedle into the ocular tissue. In addition, the microneedle device may include means of controlling the angle at which the at least one microneedle is inserted into the ocular tissue (e.g., by inserting the at least one microneedle into the surface of the ocular tissue at an angle of about 90 degrees).

In one embodiment, the depth of microneedle insertion into the ocular tissue can be controlled by the length of the microneedle, as well as other geometric features of the microneedle. For example, a flange or other a sudden change in microneedle width can be used to limit the depth of microneedle insertion. The microneedle insertion can also be controlled using a mechanical micropositioning system involving gears or other mechanical components that move the microneedle into the ocular tissue a controlled distance and, likewise, can be operated, for example, in reverse, to retract the microneedle a controlled distance. The depth of insertion can also be controlled by the velocity at which the microneedle is inserted into the ocular tissue. The retraction distance can be controlled by elastic recoil of the ocular tissue into which the microneedle is inserted or by including an elastic element within the microneedle device that pulls the microneedle back a specified distance after the force of insertion is released.

The angle of insertion can be directed by positioning the microneedle at a first angle relative to the microneedle base and positioning the base at a second angle relative to the ocular surface. In one embodiment, the first angle can be about 90° and the second angle can be about 0°. The angle of insertion can also be directed by having the microneedle protrude from a device housing through a channel in that housing that is oriented at a specified angle.

As provided throughout, in one embodiment, the methods described herein are carried out with a hollow or solid microneedle, for example, a rigid microneedle. As used herein, the term “microneedle” refers to a conduit body having a base, a shaft, and a tip end suitable for insertion into the sclera and other ocular tissue and has dimensions suitable for minimally invasive insertion and drug formulation infusion as described herein. That is, the microneedle has a length or effective length that does not exceed about 2000 microns and a diameter that does not exceed about 600 microns. Both the “length” and “effective length” of the microneedle encompass the length of the shaft of the microneedle and the bevel height of the microneedle. In some embodiments, the microneedle used to carry out the methods described herein comprises one of the devices disclosed in International Patent Application Publication No. WO2014/179698 (Application No. PCT/US2014/036590), filed May 2, 2014 and entitled “Apparatus and Method for Ocular Injection,” incorporated by reference herein in its entirety for all purposes. In some embodiments, the microneedle used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes.

In another embodiment, the microneedle is designed to have a length longer than the desired penetration depth, but the microneedle is controllably inserted only part way into the tissue. Partial insertion may be controlled by the mechanical properties of the tissue, which bends and dimples during the microneedle insertion process. In this way, as a microneedle is inserted into the tissue, its movement partially elastically deforms the tissue and partially penetrates into the tissue. By controlling the degree to which the tissue deforms, the depth of microneedle insertion into the tissue can be controlled.

In one embodiment, the device used to carry out one of the methods described herein comprises the device described in U.S. Design patent application Ser. No. 29/506,275 entitled, “Medical Injector for Ocular Injection,” filed Oct. 14, 2014, the disclosure of which is incorporated herein by reference in its entirety for all purposes.

In one embodiment, the microneedle is inserted into the eye of the human patient using a rotational/drilling technique and/or a vibrating action. In this way, the microneedle can be inserted to a desired depth by, for example, drilling the microneedles a desired number of rotations, which corresponds to a desired depth into the tissue. See, e.g., U.S. Patent Application Publication No. 2005/0137525, which is incorporated herein by reference, for a description of drilling microneedles. The rotational/drilling technique and/or a vibrating action may be applied during the insertion step, retraction step, or both.

As used herein, the words “proximal” and “distal” refer to the direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a microneedle described herein first inserted inside the patient's body would be the distal end, while the opposite end of the microneedle (e.g., the end of the medical device being manipulated by the operator) would be the proximal end of the microneedle.

As used herein, the terms “about” and “approximately” generally mean plus or minus 10% of the value stated. For example, about 0.5 would include 0.45 and 0.55, about 10 would include 9 to 11, about 1000 would include 900 to 1100.

The term “fluid-tight” is understood to encompass both a hermetic seal (i.e., a seal that is gas-impervious) as well as a seal that is only liquid-impervious. The term “substantially” when used in connection with “fluid-tight,” “gas-impervious,” and/or “liquid-impervious” is intended to convey that, while total fluid imperviousness is desirable, some minimal leakage due to manufacturing tolerances, or other practical considerations (such as, for example, the pressure applied to the seal and/or within the fluid), can occur even in a “substantially fluid-tight” seal. Thus, a “substantially fluid-tight” seal includes a seal that prevents the passage of a fluid (including gases, liquids and/or slurries) therethrough when the seal is maintained at a constant position and at fluid pressures of less than about 5 pounds per square inch gage (psig), less than about 10 psig, less than about 20 psig, less than about 30 psig, less than about 50 psig, less than about 75 psig, less than about 100 psig and all values in between. Similarly, a “substantially liquid-tight” seal includes a seal that prevents the passage of a liquid (e.g., a liquid medicament) therethrough when the seal is maintained at a constant position and is exposed to liquid pressures of less than about 5 psig, less than about 10 psig, less than about 20 psig, less than about 30 psig, less than about 50 psig, less than about 75 psig, less than about 100 psig and all values in between.

As used herein, the term “hollow” includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base.

The microneedle device in one embodiment, comprises a fluid reservoir for containing the therapeutic formulation (e.g., drug or cell formulation), e.g., as a solution or suspension, and the drug reservoir (which can include any therapeutic formulation) being in operable communication with the bore of the microneedle at a location distal to the tip end of the microneedle. The fluid reservoir may be integral with the microneedle, integral with the elongated body, or separate from both the microneedle and elongated body.

The microneedle and/or any of the components included in the embodiments described herein is/are formed and/or constructed of any suitbale biocompatible material or combination of materials, including metals, glasses, semi-conductor materials, ceramics, or polymers. Examples of suitable metals include pharmaceutical grade stainless steel, gold, titanium, nickel, iron, gold, tin, chromium, copper, and alloys thereof. The polymer can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Representative non-biodegradable polymers include various thermoplastics or other polymeric structural materials known in the fabrication of medical devices. Examples include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. Biodegradable microneedles can provide an increased level of safety compared to non-biodegradable ones, such that they are essentially harmless even if inadvertently broken off into the ocular tissue.

In one embodiment, the hollow microneedle provided herein is fabricated using a laser or similar optical energy source. In one example, a microcannula may be cut using a laser to represent the desired microneedle length. The laser may also be use to shape single or multiple tip openings. Single or multiple cuts may be performed on a single microncannula to shape the desired microneedle structure. In one example, the microcannula may be made of metal such as stainless steel and cut using a laser with a wavelength in the infrared region of the light spectrum (e.g., from about 0.7 to about 300 μm). Further refinement may be performed using metal electropolishing techniques familiar to those in the field. In another embodiment, the microneedle length and optional bevel is formed by a physical grinding process, which for example may include grinding a metal cannula against a moving abrasive surface. The fabrication process may further include precision grinding, micro-bead jet blasting and ultrasonic cleaning to form the shape of the desired precise tip of the microneedle.

Further details of possible manufacturing techniques are described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. No. 6,334,856, U.S. Pat. No. 6,611,707, U.S. Pat. No. 6,743,211 and PCT/US2014/36590, filed May 2, 2014, all of which are incorporated herein by reference in their entireties for all purposes.

In some embodiments, an apparatus includes a medicament container, a piston assembly and a handle. The medicament container defines a lumen configured to contain a medicament. A distal end portion of the medicament container includes a coupling portion configured to be removably coupled to a needle assembly. A proximal end portion of the medicament container includes a flange and a longitudinal shoulder. A distal end portion of the piston assembly includes an elastomeric member movably disposed within the lumen of the medicament container. The handle is coupled to a proximal end portion of the piston assembly such movement of the handle produces movement of the elastomeric member within the medicament container. The proximal end portion of the medicament container is movably disposed within the handle. A portion of the handle is configured to contact the flange to limit proximal movement of the handle relative to the medicament container. The handle includes a protrusion configured to engage the longitudinal shoulder of the medicament container to limit rotation of the handle relative to the medicament container.

Any of the compositions described herein can be injected using any suitable injector of the types shown and described herein. Any of the methods described herein can be performed use any suitable injector of the types shown and described herein. In this manner, the benefits of targeted drug delivery via a non-surgical approach can be realized. For example, in some embodiments, an apparatus includes a medicament container, a needle assembly, and a piston assembly. The medicament container contains a dose of a medicament, such as, for example a drug or cellular therapeutic, e.g., a steroid formulation or a cell suspension (e.g., a stem cell suspension). The dose has a delivered volume of at least about 20 μL, at least about 50 uL, at least about 100 μL, at least about 200 μL or at least about 500 μL. In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space.

The needle assembly is coupled to a distal end portion of the medicament container, and includes a contact surface and a needle. The contact surface is configured to contact a target surface of an eye, and can include a convex surface and/or a sealing portion, as described herein. The needle is coupled to the base. A distal end portion of the piston assembly includes an elastomeric member movably disposed within the medicament container. A proximal end portion of the piston assembly is configured to receive a force to move the elastomeric within the medicament container to deliver the dose of the medicament via the needle assembly. The needle assembly and the piston assembly being collectively configured to deliver the dose of the medicament into the suprachoroidal space of the eye such that an intraocular pressure of the eye measured within 30 minutes after delivery of the dose is within five percent, ten percent, fifteen percent, twenty percent or twenty-five percent of an intraocular pressure of the eye measured before the delivery of the dose.

In some embodiments, an apparatus includes a medicament container, a needle assembly, and a piston assembly. The medicament container contains a dose of a medicament, such as, for example a steroidal composition such as a triamcinolone composition. The needle assembly is coupled to a distal end portion of the medicament container, and includes a contact surface and a needle. The contact surface is configured to contact a target surface of an eye, and can include a convex surface and/or a sealing portion, as described herein. The needle is coupled to the base. A distal end portion of the piston assembly includes an elastomeric member movably disposed within the medicament container. A proximal end portion of the piston assembly is configured to receive a force to move the elastomeric within the medicament container to deliver the dose of the medicament via the needle assembly. The needle assembly and the piston assembly being collectively configured to deliver the dose of the medicament into a suprachoroidal space of the eye such that a therapeutic response resulting from the dose is substantially equivalent to a therapeutic response resulting from the delivery of a corresponding dose of the medicament via any one of an intravitreal delivery method, a topical delivery method, a parenteral delivery method or an oral delivery method. An amount of the dose is less than about 75 percent of an amount of the corresponding dose.

In some embodiments, an apparatus includes a medicament container, a needle assembly, and a piston assembly. The medicament container contains a dose of a medicament, such as, for example a steroidal composition such as a triamcinolone composition. The needle assembly is coupled to a distal end portion of the medicament container, and includes a contact surface and a needle. The contact surface is configured to contact a target surface of an eye, and can include a convex surface and/or a sealing portion, as described herein. The needle is coupled to the base. A distal end portion of the piston assembly includes an elastomeric member movably disposed within the medicament container. A proximal end portion of the piston assembly is configured to receive a force to move the elastomeric within the medicament container to deliver the dose of the medicament via the needle assembly. The needle assembly and the piston assembly being collectively configured to deliver the dose of the medicament into a suprachoroidal space of the eye such that an intraocular Cmax resulting from the dose is greater, for example at least about 1.25×, 1.5× or 2× greater than an intraocular Cmax resulting from the delivery of a corresponding dose of the medicament via any one of an intravitreal delivery method, a topical delivery method, a parenteral delivery method or an oral delivery method.

The needle assembly and the piston assembly being collectively configured to deliver the dose of the medicament into a suprachoroidal space of the eye such that an intraocular AUC resulting from the dose is greater, for example at least about 1.25×, 1.5× or 2× greater than an intraocular AUC resulting from the delivery of a corresponding dose of the medicament via any one of an intravitreal delivery method, a topical delivery method, a parenteral delivery method or an oral delivery method.

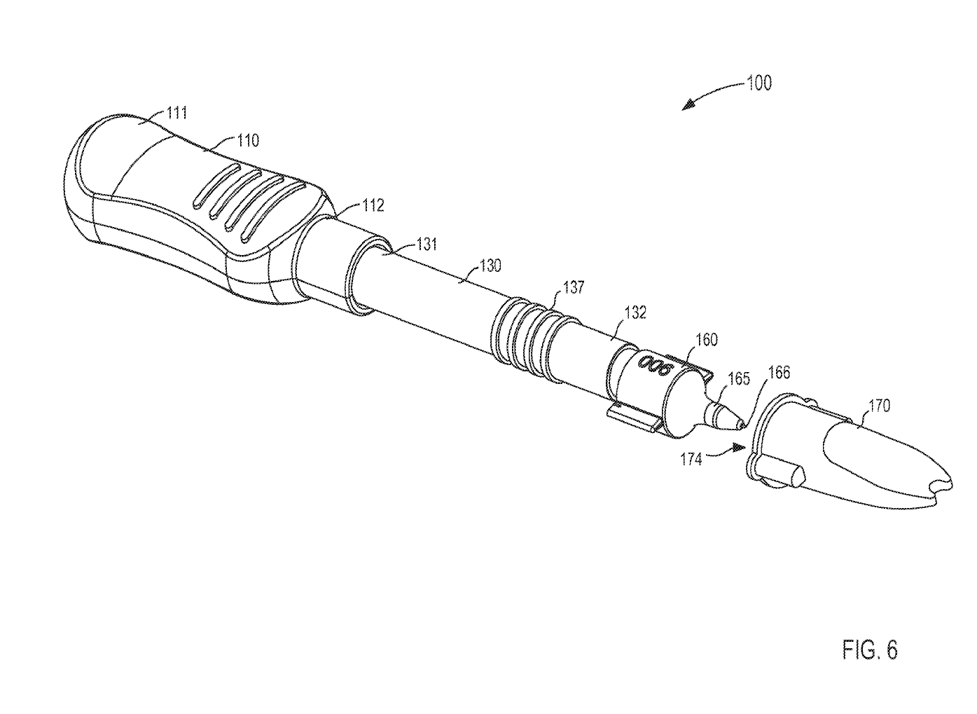

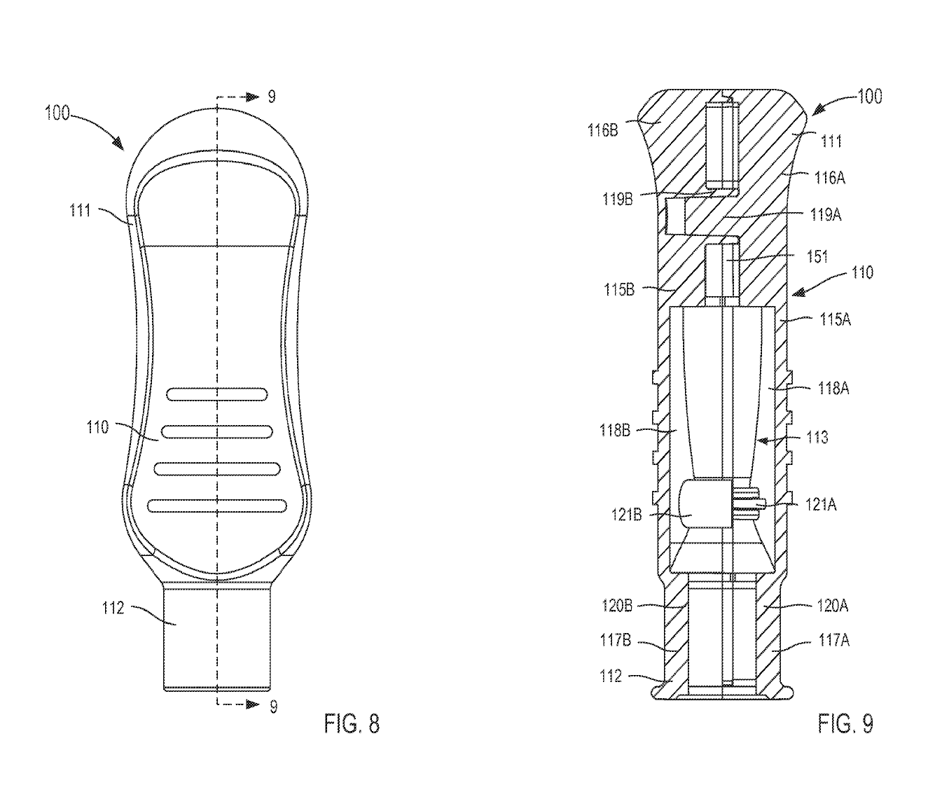

FIGS. 5-18 illustrate a medical injector 100 configured to deliver a medicament to, for example, ocular tissue, according to an embodiment. The medical injector 100 can be used in conjunction with any of the methods and therapeutic formulations described herein. More specifically, the medical injector 100 (also referred to herein as “injector”) can have a size, shape, and/or configuration that is based at least in part on constraints and/or challenges associated with delivering a drug formulation into ocular tissue. For example, as described in further detail herein, medicament delivery into ocular tissue using conventional devices and/or needles can lead to incomplete delivery of a dose, reduction in efficacy of an injected medicament, seeding of undersirable cells, trauma, etc. Thus, the medical injector 100 can have a size and/or configuration that effectively deliver a medicament to a portion of the eye such as a posterior region thereof.

As shown, the medical injector 100 includes a handle 110, a barrel 130, a piston 150, a needle hub 160, and a cap 170. The handle 110 can be any suitable shape, size, and/or configuration. For example, in some embodiments, the handle 110 can have an ergonomic shape and/or size, which can enable to manipulate the injector 100 with one hand or with two hands. The handle 110 has a proximal end portion 111 and a distal end portion 112, and defines an inner volume 113 (see e.g., FIG. 9). The inner volume 113 of the handle 110 receives and/or is configured to house at least a portion of the barrel 130 and the piston 150, as described in further detail herein.

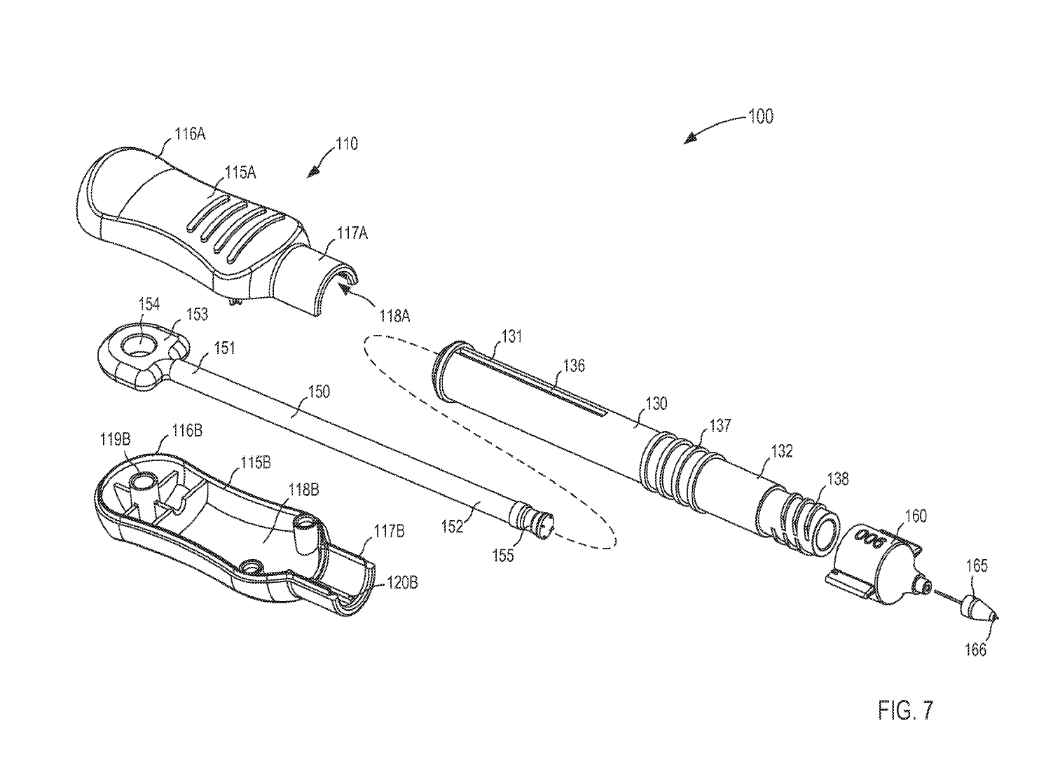

As shown in FIG. 7-9, the handle 110 is formed by coupling a first handle member 115A to a second handle member 115B. The handle member 115A and the handle member 115B can be relatively thin shelled or the like and can be formed from any suitable material such as the biocompatible materials described above. In other words, the handle members 115A and 1158B can be substantially hollow and/or can define an inner volume (e.g., the inner volume 113). The first handle member 115A has a proximal end portion 116A and a distal end portion 117A. Moreover, the first handle member 115A has an inner surface 118A that can include any suitable feature, cutout, coupler, wall, etc., any of which can be used to facilitate the coupling of the first handle member 115A to the second handle member 115B and/or to engage a portion of the piston 150 and/or the barrel 130. For example, as shown in FIG. 9, the inner surface 118A of the first handle member 115A can form a rib 120A, a retention member 119A, and at least one coupler 121A, which can be used, iner alia, to engage the barrel 130, the piston 150, and/or the second handle member 115B, respectively, as described in further detail herein.

Similarly, the second handle member 115B has a proximal end portion 116B and a distal end portion 117B. The second handle member 115B also has an inner surface 118B that forms a rib 120B, a retention member 119B, and at least one coupled 121B, which can be used to engage the barrel 130, the piston 150, and the first handle member 115A, respectively, as described in further detail herein. As shown in FIG. 9, for example, the first handle member 115A and the second handle member 115B are coupled together to collectively form the handle 100. The first handle member 115A and the second handle member 115B can be coupled in any suitable member. For example, in some embodiments, the retention member 119B of the second handle member 115B can define an opening or the like configured to matingly receive a portion of the retention member 119A of the first handle member 115A. Similarly, the at least one coupler 121B of the second handle member 119B can define an opening configured to matingly receive a portion of an associated coupler 121A of the first handle member 115A. In some embodiments, the retention member 119A and the coupler(s) 121B of the first handle member 115A can be configured to form a press or friction fit with an inner surface of the retention member 119B and the coupler(s) 121B of the second handle member 115B, which can be operable in coupling the first handle member 115A to the second handle member 115B. In other embodiments, the first handle member 115A and the second handle member 115B can be coupled via any suitable method such as, for example, an adhesive, an ultrasonic weld, a mechanical fastener, and/or the like. Furthermore, when the first handle member 115A is coupled to the second handle member 115B, the inner surfaces 118A and 118B of the handle members 115A and 115B, respectively, collectively define the inner volume 113 of the handle 110, as shown in FIG. 9.

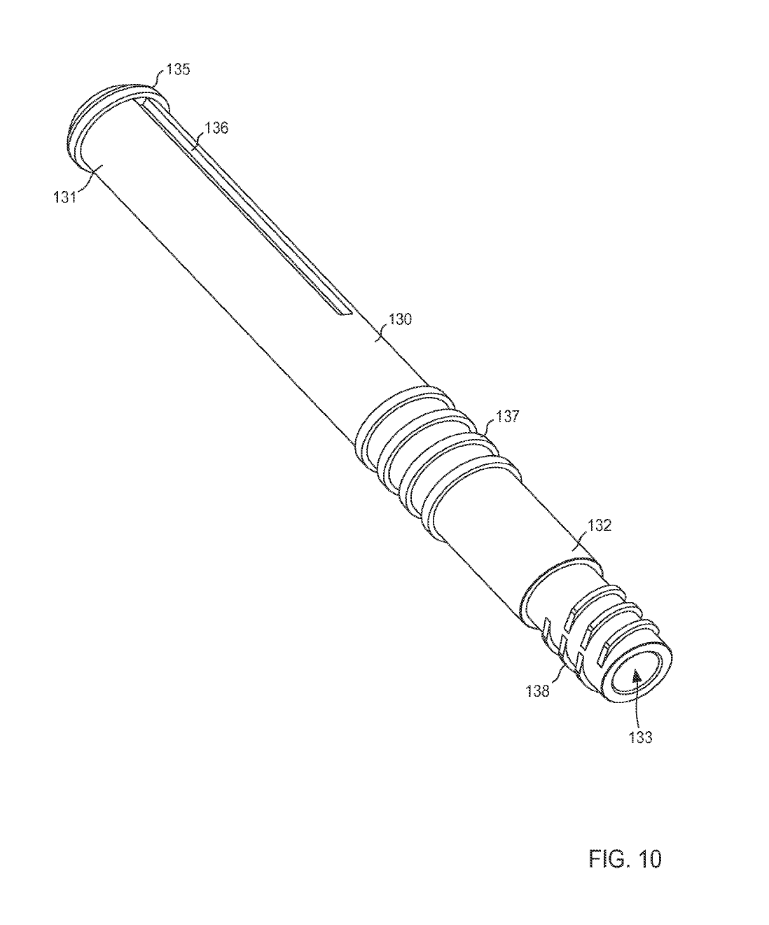

The barrel 130 of the injector 100 can be any suitable shape, size, or configuration. As shown in FIG. 10, the barrel 130 has a proximal end portion 131 and a distal end portion 132 and defines a lumen 133 therethrough. In addition, the barrel 130 has an outer surface that defines a set of slots 136 (only one is shown in FIG. 10) and a grip portion 137. The grip portion 137 can be configured to facilitate the use of the device by providing a user with a predetermined location to engage the injector 100. The grip portion 137 can have any suitable surface finish or the like, which can, in some instances, increase a friction between the grip portion 137 and a user's fingers and/or hand. In other embodiments, the barrel 130 does not include a grip portion.

The lumen 133 of the barrel 130 movably receives at least a portion of the piston 150, as described in further detail herein. Moreover, at least a portion of the lumen 133 can define a medicament volume configured to receive, store, house, and/or otherwise contain a medicament (e.g., a corticosteroid such as triamcinolone acetonide, or any other medicament described herein). In some embodiments, at least a portion of the barrel 130 can be substantially transparent and/or can include an indicator or the like configured to allow a user to visually inspect a volume of fluid (e.g., medicament/therapeutic formulation) within the lumen 133. In some instances, such an indicator can be, for example, any number of lines and/or markings associated with a volume of fluid disposed within the barrel 130. In other embodiments, the barrel 130 can be substantially opaque and/or does not include an indicator or the like.

The distal end portion 132 includes and/or forms a coupler 138 configured to be physically and fluidically coupled to the needle hub 160, as described in further detail herein. The proximal end portion 131 of the barrel 130 includes a flanged end 135 and defines a set of slots 136 (only one slot is shown in FIG. 10). As described above, at least a portion of the barrel 130 is disposed within the inner volume 113 of the handle 110 (see, e.g., FIG. 16). Specifically, at least the proximal end portion 131 of the barrel 130 can be inserted into the handle 110 in such a manner that the handle 110 can be moved relative to the barrel 130. In other words, at least the proximal end portion 131 of the barrel 130 can be movably disposed within the inner volume 113 defined by the handle 110. Moreover, when the proximal end portion 131 of the barrel 130 is disposed in the handle 110, the ribs 120A and 120B of the handle members 115A and 115B, respectively, are movably disposed in its associated slot 136 defined by the barrel 130. Such an arrangement can, for example, define a range of motion of the handle 110 relative to the barrel 130. Such an arrangement can also limit a rotational motion of the handle 110 about the barrel 130 while allowing a translational motion of the handle 110 relative to the barrel 130 in a proximal or a distal direction. In this manner, during the injection operation, substantially all of the force applied by the user will urge the handle 110 (and therefore the piston 150) in the distal direction, and will not cause rotation of the piston 150 within the barrel 130. By limiting the rotational motion of the piston 150 (and particularly, the elastomeric member 155) within the barrel 130, the injection operation can be consistently performed. For example, by limiting the rotational motion of the elastomeric member 155 within the barrel 130, the force needed to overcome the static coefficient of friction between the elastomeric member 155 and the barrel 130 will be more consistent (between parts andor injection) than if an applied force includes both translational (i.e., distal) and rotational components. This arrangement facilitates a more consistent “loss of resistance” felt at the handle 110 during an injection operation, as described below.

Additionally, the arrangement of the flanged end 135 of the barrel 130 and the inner surfaces 118A and 118B of the handle members 115A and 115B, respectively, can define a translational range of motion of the handle 110 relative to the barrel 130 in the proximal or the distal direction (see e.g., FIG. 16).

The piston 150 of the injector 100 can be any suitable shape, size, and/or configuration. For example, referring back to FIG. 7, the pison 150 can have a size and shape that are each associated with the handle 110 and/or the barrel 130, which in turn, can allow at least a portion of the piston 150 to be disposed within the handle 110 and/or the barrel 130. More specifically, the piston 150 has a proximal end portion 151 and a distal end portion 152. The proximal end portion 151 of the piston 150 is configured to be disposed within the inner volume 113 of the handle 110. As shown in FIG. 7, the proximal end portion 151 of the piston 150 includes a tab 153 or the like that defines an opening 154, which in turn, can receive at least a portion of the retention members 119A and 119B of the handle members 115A and 115B, respectively. For example, in some embodiments, during an assembly and/or manufacturing process and prior to coupling the handle members 115A and 115B, the proximal end portion 151 of the piston 150 can be positioned relative to the retention member 119B of the second handle member 115B such that at least a portion of the retention member 119B is disposed within the opening 154 defined by the piston 150. In other words, the tab 153 at or near the proximal end portion 151 of the piston 150 can be disposed about a portion of the retention member 119B prior to coupling the first handle member 115A to the second handle member 115B. As such, the piston 150 can be fixedly coupled to the handle 110.

The distal end portion 152 of the piston 150 is configured to be movably disposed in the lumen 133 of the barrel 130. As shown in FIG. 7, the distal end portion 152 of the piston 150 includes and/or is coupled to an elastomeric member 155. In some embodiments, the elastomeric member 155 can be monolithically formed with the piston 150 (e.g., overmolded or the like). In other embodiments, the elastomeric member 155 can be formed independently of the piston 150 and coupled thereto. The elastomeric member 155 can be made of an inert and/or biocompatible material, which can have any suitable hardness and/or durometer. For example, in some embodiments, the elastomeric member 155 can be formed from and/or constructed out of a rubber, silicone, plastic, nylon, polymers, any other suitable material or combination thereof. In some embodiments, at least a portion of the elastomeric member 155 can be configured to deform or the like while substantially maintaining its original shape. That is to say, the elastomeric member 155 can have a durometer that is sufficiently low to allow at least some deformation thereof, while preventing the elastomeric member 155 from being substantially reconfigured and/or the like.

The elastomeric member 155 can be disposed in the lumen 113 such that an outer surface of the elastomeric member 155 is in contact with an inner surface of the barrel 130 defining the lumen 133. In some embodiments, the elastomeric member 155 and the inner surface of the barrel 130 collectively form a substantially fluid-tight seal and/or a hermetic seal, which can, for example, prevent leakage, out gassing, contamination, and/or the like of a substance (e.g., a medicament) disposed within the barrel 130. Moreover, the elastomeric member 155 can have a size, shape and/or can be constructed from a material such that movement of the piston 150 and/or elastomeric member 155 within the barrel 130 is limited when a force applied is below a predetermined threshold. In this manner, the piston 150 can be maintained in a substantially fixed position relative to the barrel 130 until a force exerted, for example, on the handle 110 is sufficient to inject a medicament into a target tissue, as described in further detail herein. In some embodiments, the size, shape, and/or configuration of the elastomeric member 155 can be changed to, for example, increase or decrease an amount of force used to move the piston 150 within the barrel 130, which in some instances, can be based on one ore more characteristics associated with a target tissue and/or the like, as described in further detail herein.

The needle hub 160 of the injector 100 can be any suitable shape, size, and/or configuration. As shown in FIGS. 11-13, 15, and 16, the needle hub 160 has a proximal end portion 161, a distal end portion 162, an indicator portion 168, and a pair of tabs 164, and defines a lumen 167 (see e.g., FIG. 16). The proximal end portion 161 of the needle hub 160 is configured to be coupled to the distal end portion 132 of the barrel 130. For example, the needle hub 160 can include a coupler 163 (see e.g., FIG. 16) that can matingly engage the coupler 138 of the barrel 130 to couple the needle hub 160 to the barrel 130 and to place the lumen 167 of the needle hub 160 in fluid communication with the lumen 133 of the barrel 130. In some embodiments, the coupler 163 of the needle hub 160 and the coupler 138 of the barrel 130 can form a threaded coupling or the like. In such embodiments, a user can, for example, engage the tabs 164 to rotate the needle hub 160 relative to the barrel 130, thereby threading the coupler 163 of the needle hub 160 onto the coupler 138 of the barrel 130. In some embodiments, the coupler 163 of the needle hub 160 can be a locking mechanism and/or the like such as, for example, a Luer-Lok® (or other locking mechanism) configured to form a fluid tight seal with the distal end portion 132 of the barrel 130 when coupled thereto. The distal end portion 162 of the needle hub 160 includes and/or is coupled to a base 165, which in turn, is coupled to and/or forms a microneedle 166, as described below. The indicator portion 168 of the needle hub 160 is configured to provide a visual indication associated with one or more characteristics of the microneedle 166. For example, in this embodiment, the indicator portion 168 can be configured to provide a visual indication associated with an effective length of the microneedle 166 (e.g., “900” micrometers, as shown in FIG. 12).

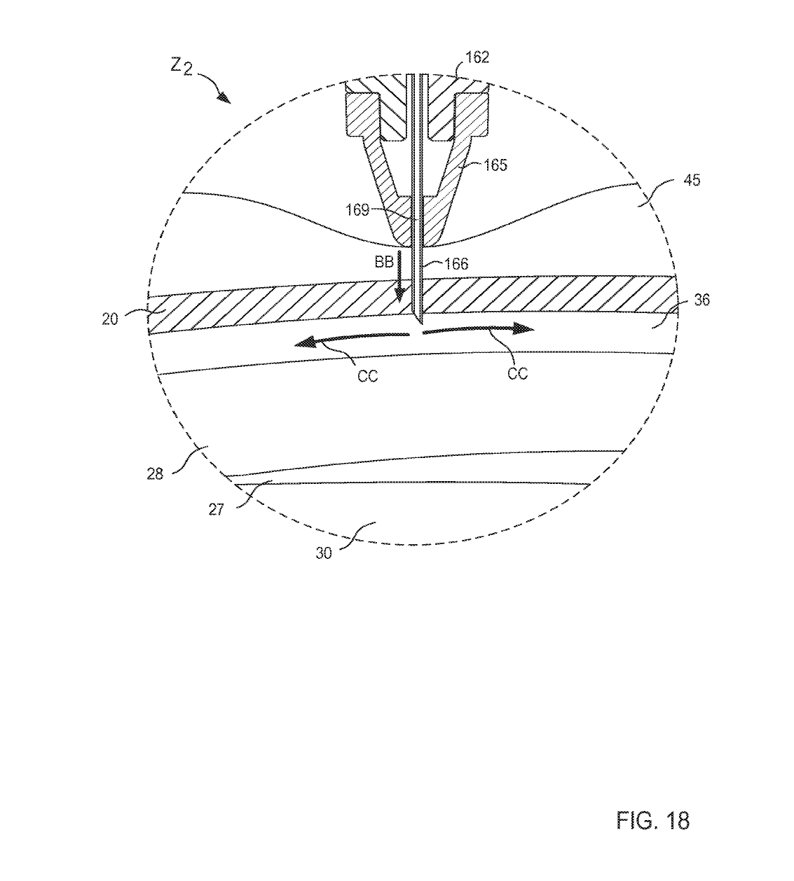

The base 165 can be any suitable shape, size, and/or configuration and can be configured to contact a portion of the ocular tissue during an injection event. For example, as shown, the base 165 has a convex distal end surface, which is configured to contact a target surface of a target tissue when a substance is conveyed through the needle into the target tissue (see, e.g., FIG. 18). In some embodiments, the distal end surface includes a sealing portion (not identified in the FIGS.) configured to define a substantially fluid-tight seal with the target surface when the distal end surface is in contact with the target surface. For example, the distal end surface of the base 165 can deform the target surface such that the sealing portion is contiguous with the target surface and forms the substantially fluid-tight seal. In some embodiments, the sealing portion can be symmetrical about the microneedle 166.

In some embodiments, the base 165 can be formed from a material or combination of materials that is/are relatively flexible and/or that has/have a relatively low durometer. In some instances, the base 165 can be formed from a material with a durometer that is sufficiently low to limit and/or prevent damage to the ocular tissue when placed in contact therewith. In some instances, the base 165 can be configured to deform (e.g., elastically or plastically) when placed in contact with the ocular tissue. In other embodiments, the base 165 can be formed from a material of sufficient hardness such that the target tissue (and not the base) is deformed when the base 165 is placed in contact with and/or pressed against the target tissue. In some embodiments, for example, the base 165 is constructed from a medical grade stainless steel, and has a surface finish of less than about 1.6 μm Ra. In this manner, the surface finish can facilitate the formation of a substantially fluid-tight seal between the base 165 and the target tissue.

Furthermore, when the base 165 is coupled to the needle hub 160, a lumen 169 defined by the microneedle 166 is in fluid communication with the lumen 167 of the needle hub 160 (see, e.g., FIG. 16). Thus, a substance can flow through the lumen 167 of the needle hub 160 and the lumen 169 of the microneedle 166 to be injected into a target tissue, as described in further detail herein.

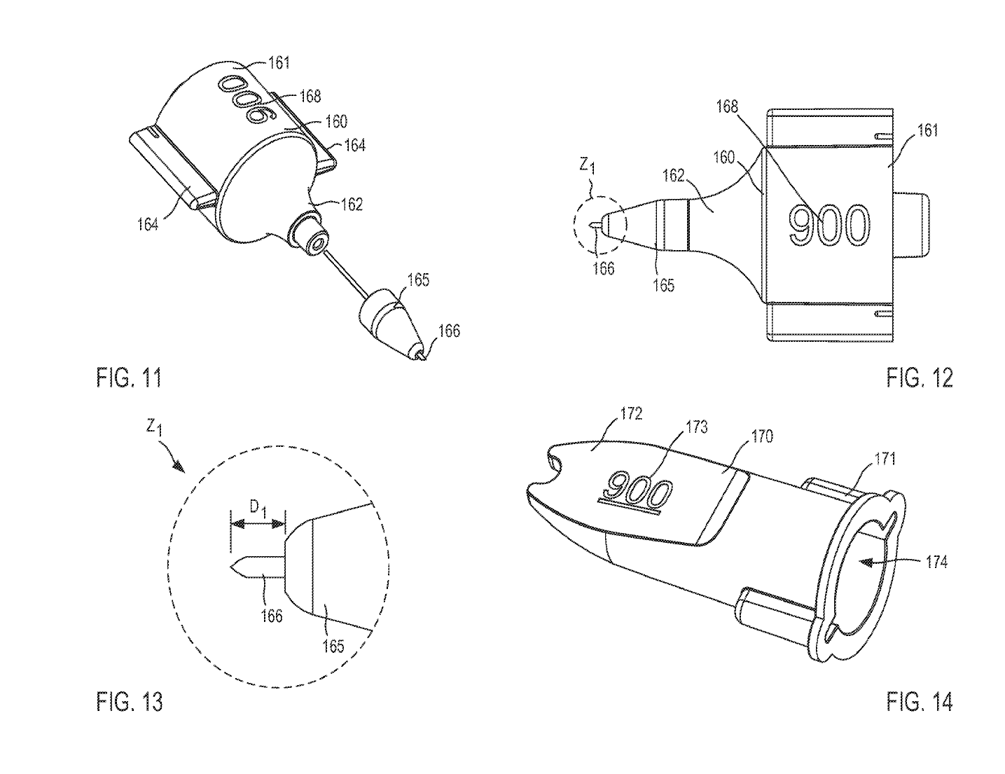

The microneedle 166 can be any suitable device or structure that is configured to puncture a target tissue of a patient. For example, the microneedle 166 can be any of the microneedles described herein configured to puncture ocular tissue. In some embodiments, the microneedle 166 can be a 30 gauge microneedle, a 32 gauge microneedle or a 34 gauge microneedle. As shown in FIG. 13, the microneedle 166 extends from a distal surface of the base 165 by a distance D₁ (also referred to herein as an “effective length”). In some embodiments, the shape and/or size of the microneedle 166 can correspond with at least a portion of a target tissue. For example, in some embodiments, the effective length of the microneedle 166 (e.g., the portion of the microneedle 166 that is outside or distal to the base 165) can correspond with a portion of ocular tissue such that when the microneedle 166 is inserted into the ocular tissue, a portion of the microneedle 166 is disposed within the sclera or suprachoroidal space of the eye. Specifically, in this embodiment, the effective length and/or the distance D₁ is about 900 micrometers (nm). Moreover, the indicator portion 168 of the needle hub 160 can be configured to provide a user with a visual indication associated the effective length and/or distance D₁. Although not shown in FIGS. 11-13, in some embodiments, the microneedle 166 can have a bevel geometry (e.g., bevel angle, bevel height, bevel aspect ratio or the like), which can facilitate the piercing and/or insertion of a tip of the microneedle 166 into the target tissue and the opening (not shown) of the microneedle 166 can be maintained within a desired region during an injection event. In some embodiments, the microneedle 166 or any of the microneedles described herein can include a bevel or other characteristics of the types shown and described in International Patent Application Publication No. WO2014/036009 (Application No. PCT/US2013/056863), filed Aug. 27, 2013 and entitled “Apparatus and Method for Drug Delivery Using Microneedles” and/or International Patent Application Publication No. WO2014/179698 (International Application No. PCT/US2014/036590), filed May 2, 2014 and entitled “Apparatus and Method for Ocular Injection,” each of which is incorporated by reference herein in its entirety for all purposes.

As described above, the base 165 can be coupled to the needle hub 160, which in turn, is coupled to the barrel 130 such that the lumen 133 of the barrel, the lumen 167 of the needle hub 160, and the lumen 169 of the microneedle 166 define a fluid flow path through which a medicament and/or substance contained within the barrel 130 can flow, for example, to be injected into a target tissue.

The cap 170 of the injector 100 is removably disposed adjacent to a distal end portion 132 of the barrel 130 and is configured to substantially house, cover, enclose, protect, isolate, etc. at least a portion of the needle hub 160. More specifically, the cap 170 can be moved relative to the remaining portions of the medical injector 100 to position at least a portion of the needle hub 160 within an inner volume 174 (see, e.g., FIG. 14) of the cap 170. As such, the cap 170 can have a size and/or shape that is associated with and/or at least partially based on a size and/or shape of the needle hub 160. In some embodiments, the cap 170 and a portion of the needle hub 160 can collectively define a friction fit or the like, which can be operable in maintaining the cap 170 in a substantially fixed position relative to the needle hub 160. In addition, in some embodiments, the cap 170 and the portion of the needle hub 160 can collectively form a substantially fluid tight and/or substantially hermetic seal, which in turn, can maintain the sterility of a microneedle 166 prior to use of the medicament delivery device 100. For example, although not shown, the cap 170 can include a plug, a seal, a sterilization member (e.g., wipe, pad, etc.), and/or the like configured to maintainen the sterility of the microneedle 166 prior to use. Moreover, as shown in FIG. 14, the cap 170 includes an indicator portion 173 that can provide a visual indication to a user associated with a size and/or effective length of the microneedle 166. In some embodiments, the indicator portion 173 can be substantially similar in form and function to the indicator portion 168 of the needle hub 160 and can be configured to provide substantially the same visual indication.

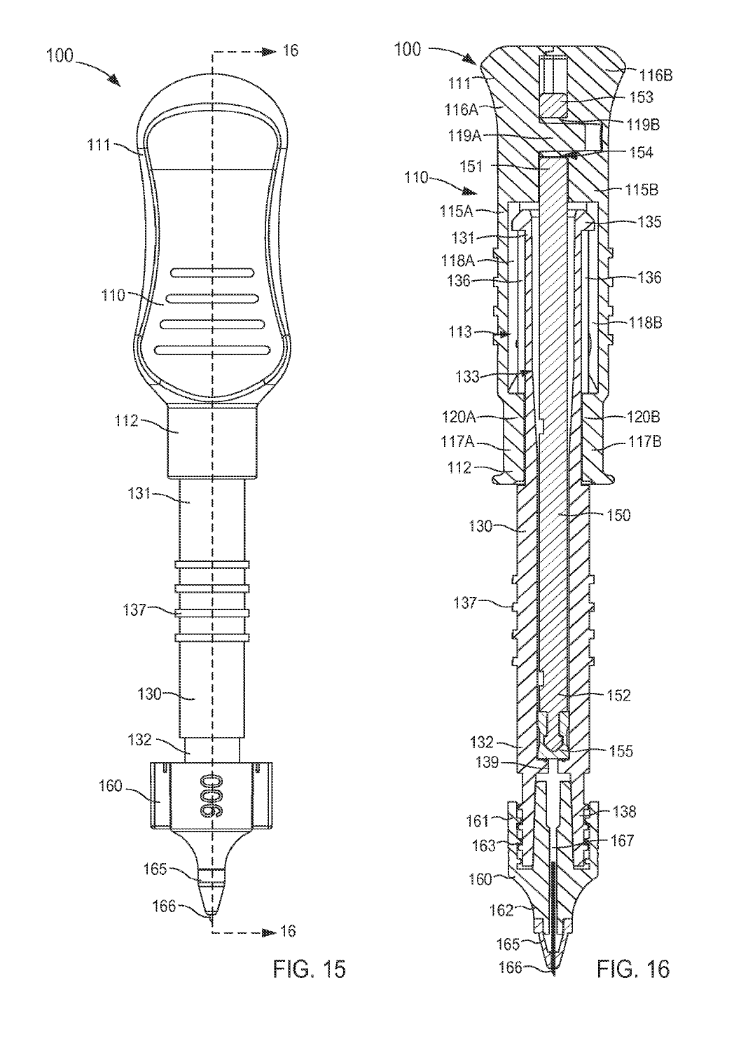

As shown in FIGS. 15-18, in some instances, a user (e.g., a doctor, technician, nurse, physician, ophthalmologist, etc.) can manipulate the injector 100 to deliver a drug formulation to the suprachoroidal space of an eye according to an embodiment. In some instance, prior to an injection event, the user can, for example, couple the distal end portion 132 of the barrel 130 to a fluid reservoir or the like and/or any suitable transfer device (not shown) to transfer a volume of a medicament and/or drug formulation into the lumen of the barrel 130. For example, in some embodiments, the distal end portion 132 of the barrel 130 can be physically and fluidically coupled to a transfer adapter and/or the like having a puncture member configured to puncture a fluid reservoir containing a drug formulation such as those described herein. Such transfer adapters can be similar to the adapter 21280 shown and described in International Patent Application Publication No. WO2014/179698 (Application No. PCT/US2014/036590), filed May 2, 2014 and entitled “Apparatus and Method for Ocular Injection,” incorporated by reference herein in its entirety for all purposes. As such, the puncture member places the transfer adapter in fluid communication with the fluid reservoir. With the transfer adapter physically and fluidically coupled to the barrel 130, the transfer adapter similarly places the lumen 133 of the barrel 130 in fluid communication with the fluid reservoir.

With the barrel 130 in fluid communication with the fluid reservoir (not shown), the user can manipulate the injector 100 by moving the handle 110 relative to the barrel 130 in the proximal direction, which in turn, moves the piston 150 disposed within the lumen 133 of the barrel 130 in the proximal direction. As such, a volume associated with a portion of the lumen 133 defined by the barrel 130 distal to the elastomeric member 155 of the piston 150 increases and a volume associated with a portion of the lumen 133 proximal to the elastomeric member 155 decreases. In some embodiments, the friction fit and/or fluidic seal defined between the elastomeric member 155 and the inner surface of the barrel 130 can be such that the proximal movement of the piston 150 (e.g., the increase in volume of the portion of the lumen 133 distal to the elastomeric member 155) produces a negative pressure differential within the portion of the lumen 133, which can be operable in drawing a volume of the medicament and/or the drug formulation from the fluid reservoir and into the portion of the lumen 133 distal to the elastomeric member 155 (e.g., a medicament volume). In some embodiments, a predetermined volume of the drug formulation can be drawn into the lumen 133 of the barrel 130. In other embodiments, the volume of the drug formulation drawn into the lumen 133 is not predetermined. With the desired amount of drug formulation contained in the barrel 130, the user can, for example, decouple the barrel 130 from the transfer adapter (not shown). Moreover, in some embodiments, the coupler 138 and/or the distal end portion 132 of the barrel 130 can include a self-sealing port and/or any other suitable port configured to fluidically isolate the lumen 133 of the barrel 130 from a volume outside of the barrel 130. Although described above as transferring a volume of the drug formation from the fluid reservoir and into the lumen 133 of the barrel 130, in other embodiments, the injector 100 can be prefilled during, for example, a manufacturing process and/or any other time prior to use.

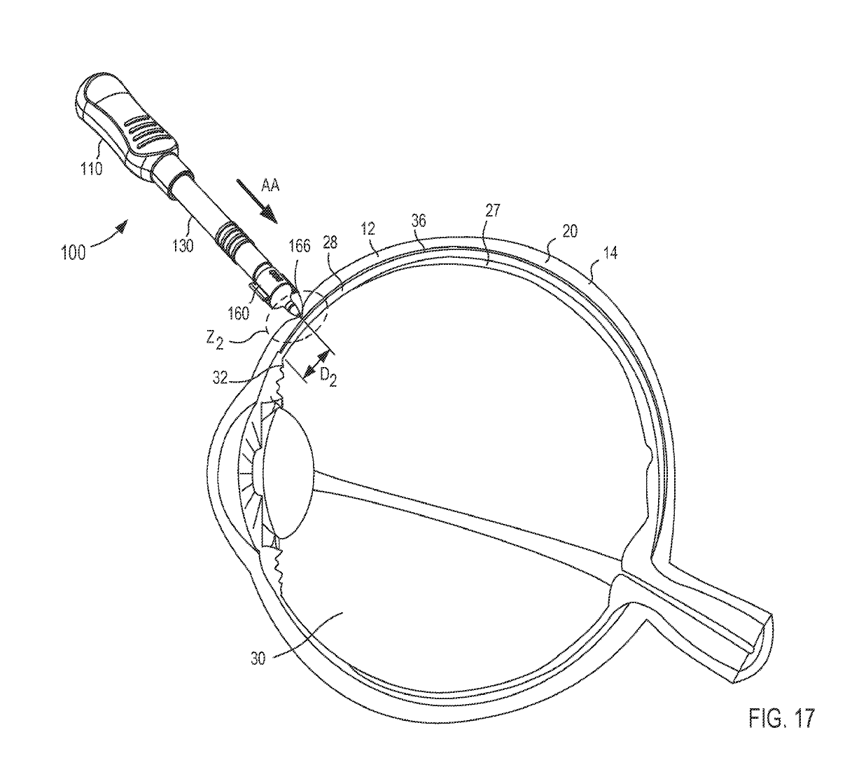

In some instances, with the desired amount of the drug formulation contained in the barrel 130, the user can manipulate the injector 100 to couple the needle hub 160 (e.g., disposed within the cap 170 or not disposed within the cap 170) to the distal end portion 132 of the barrel 130, thereby placing the lumen 169 of the microneedle 166 in fluid communication with the lumen 133 of the barrel 130. With the needle hub 160 coupled to the barrel 130, the user can remove the cap 170 from the needle hub 160 if it is disposed thereabout. In other instances, the cap 170 can already be removed. As such, the user can position the injector 100 relative to the ocular tissue such that the microneedle 166 disposed at or near a desired injection site. In some instances, the injection site can be a predetermined distance from, for example, the limbus 32. For example, as shown in FIG. 17, the injection site can be a distance D₂ from the limbus 32 that is about 1 mm, about 2 mm, about 3 mm, about 4 mm, about 5 mm, about 6 mm, about 7 mm, about 8 mm, about 9 mm, about 10 mm, or more. In other instances, an injection site can be relative to any suitable portion of the eye.

With the microneedle 166 at or near the desired injection site, the base 165 of the needle hub 160 can be pressed against a target surface of the eye 10 as the microneedle 166 is inserted into the target surface. As such, the base 165 of the needle hub 160 can deform, define an indent, and/or otherwise form a “dimple” in the target surface (e.g., the conjunctiva 45 of the eye 10, as shown in FIG. 18). The “dimple” can facilitate a desired transfer of the medicament from the barrel 130 to the target region via the microneedle 166. The base 165 of the needle hub 160, and thus the dimple, can be maintained in such a position throughout the procedure (e.g., the injection of medicament into a SCS 36). In this manner, the “dimple” (e.g., the interface between the distal surface of the base 165 and the surface of the target location) can limit and/or prevent seepage of the medicament from the target region during injection and post-injection, thereby promoting desirable transfer of the medicament to the target region (e.g., the SCS 36). As described above, in some embodiments, the distal (or contact) surface of the base 165 can include a sealing portion, which can be a convex surface, a surface having a smooth finish (e.g., with a surface finish of less than Ra=1.6 μm) or the like.

In addition, in some embodiments, the microneedle 166 is inserted substantially perpendicular or at an angle from about 80° to about 100°, into the eye 10, reaching the suprachoroidal space in a short penetration distance (e.g., about 1.1 mm, about 1 mm, about 0.9 mm, or less). This is in contrast to long conventional microneedles 166 or a cannula, which approach the suprachoroidal space at a steep angle, taking a longer penetration path through the sclera 20 and other ocular tissues, increasing the invasiveness of the method, the size of the microneedle track and consequently increasing the risk of infection and/or vascular rupture. With such long microneedles 166, the ability to precisely control insertion depth is diminished relative to the micromicroneedle 166 approach described herein.

Once the distal end portion of the microneedle 166 is disposed within at least one of the SCS 36, a lower portion of the sclera 20, and/or an upper portion of the choroid 28 of the eye 10 (FIG. 18), the medicament can be conveyed from the barrel 130. More specifically, while maintaining the dimple at the conjunctiva 45, a user can exert a force on the handle 110 to begin an infusion event. In some intsances, such as during insertion, the force exerted by a user on the handle 110 can be insufficient to move the piston 150 within the barrel 130 when the distal tip of the microneedle 166 is not disposed within the desired position (e.g., when the microneedle 166 is in the sclera 20 and not the SCS 36 of the eye 10). Said another way, the injector 100 can be configured to assist a user in delivering at least a portion of the drug formulation to the region, while be configured or “calibrated” to limit and/or prevent delivery to another, different region.

In some embodiments, the injector 100 can be configured to inform the user when the distal tip of the microneedle 166 is in the target region, for example, such that the drug formulation can be delivered to the target region with high confidence. For example, the injector 100 can be configured to limit movement of the piston 150 within the lumen 133 of the barrel 130 when the distal tip of the microneedle 166 is disposed within a region of the eye 10, which has a greater density, such as the sclera 20. In some instances, the injector 100 can limit movement of the piston 150 within the lumen 133 when the applied force is below a predetermined threshold such as about 6 Newtons (N). Conversly, the injector 100 can allow movement of the piston 150 within the barrel 130 when the distal tip of the microneedle 166 is disposed within the target location (e.g., a region having a lower density, such as the SCS 36) and when the force having the magnitude of less than about 6 N is exerted on the piston 150 and/or the handle 110. In this manner, the system can be configured or “calibrated” to provide feedback (e.g., tactile feedback) to a user to allow the user to deliver the drug formulation to a target region with high confidence. In some instances, the user can observe movement, or lack of movement, of the piston 150 within the barrel 130 to determine whether medicament has been conveyed to the eye. If the medicament has not been conveyed, the user can respond accordingly. For example, the user can re-align the system, relocate to a different injection site, and/or use a different sized microneedle 166 (e.g., a different microneedle 166 length).

By way of example, a user can manipulate the injector 100 to insert the microneedle 166 into the eye 10 at a desired injection site. In some instances, if the distal tip of the microneedle 166 is not disposed in the desired position and is, instead, disposed in the sclera 20, a force exerted by the user on the handle 110 can be insufficient to move the piston 150 within the barrel 130. For example, the sclera 20 can produce a backpressure that, in conjunction with the friction between the elastomeric member 155 and the inner surface of the barrel 130 and resistance to flow caused by the characteristcs of the drug (e.g., viscosity, density or the like), overcomes the force exerted by the user, thereby preventing and/or limiting delivery of the drug formulation to the sclera 20. In other words, the injector 100 is specifically configured or “calibrated” such that the force is insufficient to convey the drug formulation to the sclera 20. Conversely, when the distal tip of the microneedle 166 is disposed in, for example, the SCS 36 of the eye 10, the same force exerted by the user can be sufficient to move the piston 150 within the barrel, based at least in part on anatomical differences and/or the differences in material properties between the sclera 20 and the SCS 36 (e.g., densities or the like). In other words, the force can be sufficient to overcome a backpressure produced by the SCS 36. In this manner, the injector 100 can be configured to ensure that the injection is initiated only when the distal tip of the microneedle 166 is in and/or near the SCS 36 such that the drug formulation (e.g., a medicament such as, for example, a corticosteroid (e.g., triamcinolone) VEGF inhibitor, a combination thereof, or any other medicament described herein) can be delivered only to that region. Moreover, the SCS 36 produces a first pressure that resists and/or opposes flow from the distal tip of the microneedle 166, and the sclera 20 produces a second pressure that resists and/or opposes flow from the distal tip of the microneedle 166, which is higher than the first pressure. In this manner, a user can be informed by a loss of resistance felt at the handle 110 when the distal tip of the microneedle 166 is transitioned from the sclera 20 to or near the SCS 36.

In some embodiments, the force exerted can be about 2 N, about 3 N, about 4 N, about 5 N, about 6 N or more and inclusive of all ranges therebetween. In some embodiments, the piston 150 and the barrel 130 can be collectively configured such that the force produces an injection pressure within the barrel 130 of between about 100 kPa and about 500 kPa. For example, in some embodiments, the injection pressure can be about 100 kPa, 110 kPa, 120 kPa, 130 kPa, 140 kPa, 150 kPa, 160 kPa, 170 kPa, 180 kPa, 190 kPa, 200 kPa, 220 kPa, 240 kPa, 260 kPa, 280 kPa, 300 kPa, 320 kPa, 340 kPa, 360 kPa, 380 kPa, 400 kPa, 420 kPa, 440 kPa, 460 kPa, or about 480 kPa, inclusive of all ranges and values therebetween. The injection pressure can be sufficient to overcome the backpressure produced by SCS 36, but insufficient to overcome the backpressure produced by the sclera 20. In some embodiments, the force can be varied depending on the diameter of the barrel 130 and/or the piston 150, the viscosity of the drug formulation, and/or the material of the barrel 130 and/or the piston 150. In this manner, regardless of the variations in the piston 150, the barrel 130, and/or the drug formulation, the injector 100 produces an injection pressure within the barrel 130 of between about 100 kPa and about 500 kPa.

In some embodiments, the injector 100 can be configured such that injection distance traversed by the piston 150 is sufficient to deliver substantially the entire desired dose of the drug formulation into the SCS 36. In other embodiments, the injector 100 can be configured such that the injection distance traversed by the piston 150 is sufficient to deliver only a portion of the desired dose of the drug formulation into the SCS 36. In such embodiments, the injector 100 can be configured to initiate delivery of the drug formulation into the SCS 36, for example, to inform the user that the distal tip of the microneedle 166 is disposed within the SCS 36 (e.g., the user would see or otherwise detect that the piston 150 has moved, thus indicating the desired positioning of the microneedle 166). Said another way, the injector 100 can assist the user in determining whether the distal tip of the microneedle 166 is within the SCS 36 or not by initiating delivery of the drug formulation. In such embodiments, the injection distance can be a first injection distance. The user can then move the distal end portion of the piston 150 a second injection distance, for example, by applying a manual force on the piston 150 (e.g., by moving the handle 110 relative to the barrel 130, as described herein).

After desirable conveyance of the medicament from the medicament container, the hub 160 can be maintained in contact with the target surface for a time to allow for a desired medicament absorption by the eye. In this manner, the medicament can spread through tissues of the back of the eye without the medicament seeping from the injection site (e.g., where the microneedle 166 pierced the conjunctiva). As described above, in some embodiments, the distal end surface of the base 165 can include a sealing portion configured to form a substantially fluid-tight seal with the conjunctiva to limit movement of the medicament out of the eye along the needle track. In this manner, the injector 100 and the methods described herein can facilitate delivery of the desired dose to the desired regions of the eye.

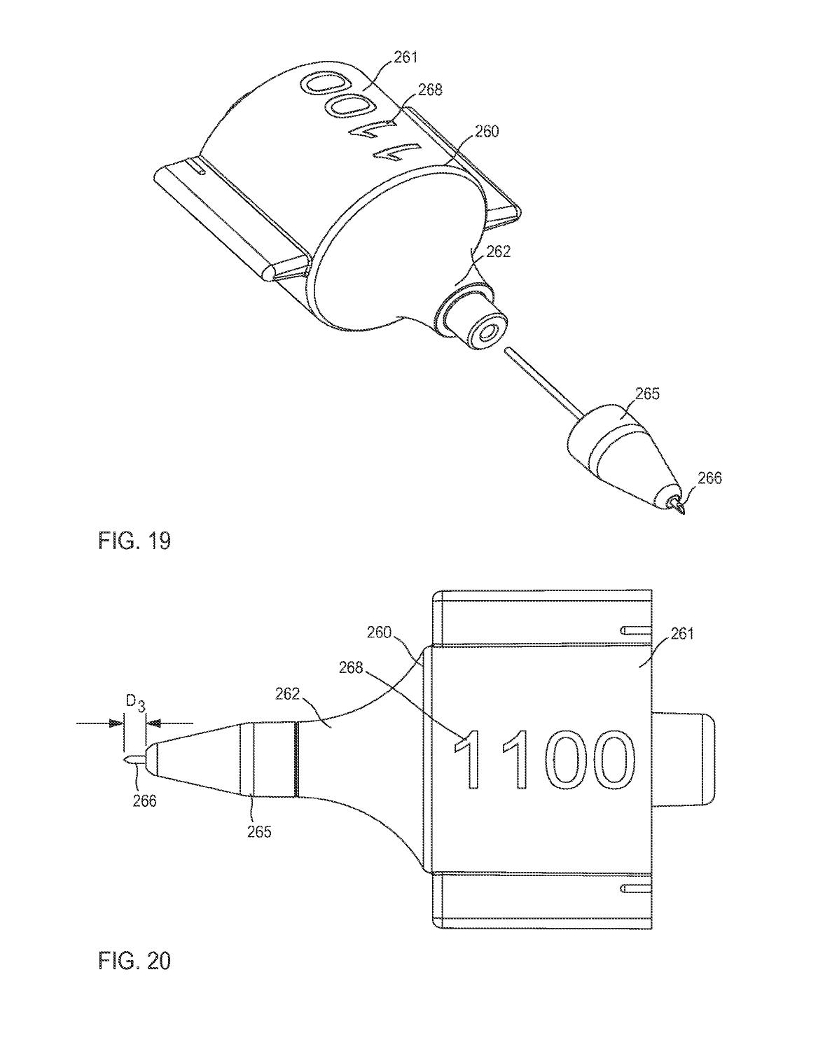

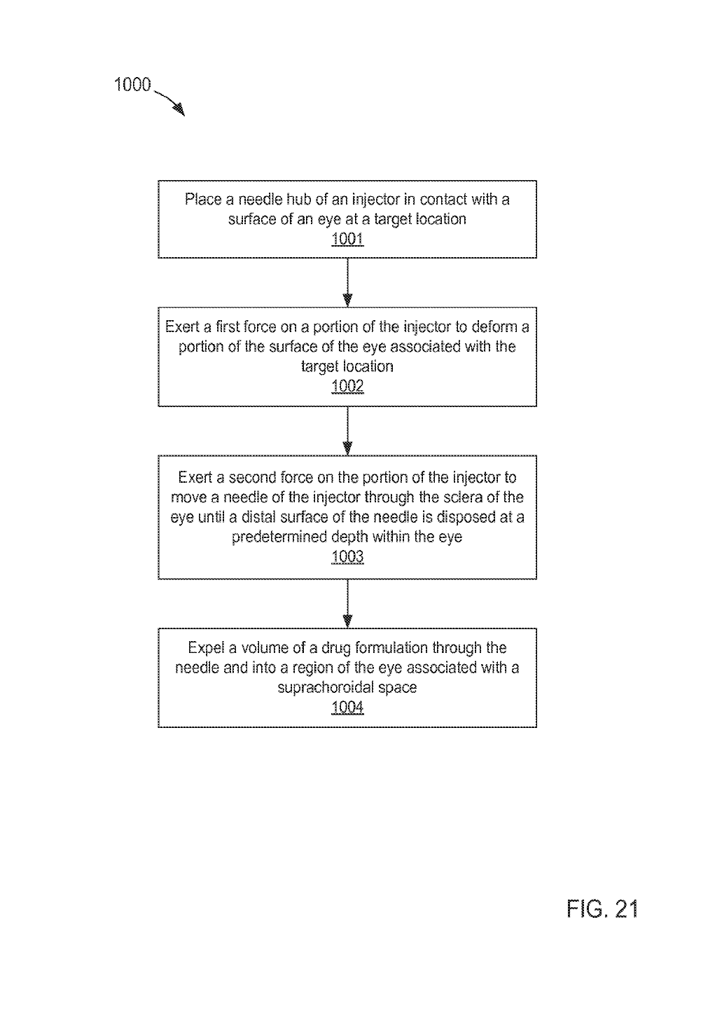

Although the microneedle 166 is described above as having an effective length that is about 900 μm, in other embodiments, the injector 100 can be coupled to a needle hub that includes a microneedle with any suitable effective length. For example, FIGS. 19 and 20 illustrate a needle hub 260 according to another embodiment. The needle hub 260 has a proximal end portion 261, a distal end portion 262, and an indicator portion 268. In some embodiments, the needle hub 260 can be substantially similar in form and function as the needle hub 160 described in detail above with reference to FIGS. 11-13. Thus, portions of the needle hub 260 are not described in further detail herein. The needle hub 260 can differ, however, by being coupled to a base 265 including a microneedle 266 with an effective length greater than the effective length of the microneedle 160. For example, in this embodiment, the microneedle 266 extends from the base 265 by a distance D₃ of about 1100 μm. Moreover, the indicator portion 268 of the needle hub 260 is configured to present a visual indication associated with the effective length and/or distance D₃ (e.g., represented in FIGS. 19 and 20 with the text “1100”).

In yet other embodiments, an injector can include a microneedle having an effective length of between about 200 μm and about 1500 μm. A short effective length microneedle (e.g., a length of between about 200 μm and about 400 μm) can be used, for example, in various subdermal injection procedures. Injectors with a longer effective length microneedle (e.g., a length of between about 1200 μm and about 1500 μm) can be used, for example, in various ocular procedures, such as, injection into the subretinal space.

Referring now to FIG. 21, a flowchart is shown illustrating a method 1000 of using a medical injector to deliver a drug formulation to ocular tissue according to an embodiment. The method 1000 includes placing a needle hub of an injector in contact with a surface of an eye at a target location, at 1001. The medical injector (also referred to herein as “injector”) can be any suitable injector. For example, in some embodiments, the injector can be substantially similar to or the same as the injector 100 described above. As such, the injector can include at least a handle, a barrel, a piston, and the needle hub. As described above, the piston can be at least partially disposed in the handle and fixedly coupled thereto. A portion of the barrel can be movably disposed in the handle to allow for relative movement, for example, in a proximal or distal direction direction. The barrel can define a lumen configured to movably receive a portion of the piston and that can receive, store, and/or contain a volume of a drug formulation. The needle hub can be coupled to the barrel to place a lumen of a microneedle coupled thereto, in fluid communication with the lumen of the microneedle.

A first force is exerted on a portion of the injector to deform a portion of the surface of the eye associated with the target location, at 1002. For example, in some instances, a user can align the injector with a target location along the surface of the eye and can move the injector to insert the microneedle into the eye and to place the needle hub in contact with a surface of the eye. The user can then exert the first force on the handle, and in response, at least a portion of the first force is transferred from the needle hub to the surface of the eye. For example, in some instances, the needle hub can exert on the conjunctiva, which can result in a dimple being formed in the conjunctiva. In some instances, the needle hub can remain in contact with the eye and can continue to deform the portion of the eye until after an injection event, which in turn, can prevent seepage and/or the like.

A second force is exerted on the portion of the injector to move a needle (e.g., the microneedle) of the injector through the sclera of the eye until a distal surface of the needle is disposed at a predetermined depth within the eye, at 1003. In some embodiments, the arrangement of the injector can be such that prior to the distal surface of the needle being disposed at the predetermined depth, the second force exerted on the portion of the injector is sufficient to move the needle through the ocular tissue, but insufficient to move the piston within the barrel. For example, in some embodiments, the piston can include an elastomeric member (e.g., a plunger or the like) that can form a friction fit with an inner surface of the barrel, which in turn, can define a reaction force that resists the movement of the piston within the barrel. Moreover, in some instances, the ocular tissue exerts a backpressure or the like in response to the insertion of the needle. As such, the amount of force exerted to move the needle through the ocular tissue (e.g., the sclera) can be less than an amount of force to move the piston within the barrel and/or otherwise inject the drug formulation.

A volume of a drug formulation is expelled through the needle and into a region of the eye associated with a suprachoroidal space, at 1004. In some instances, the region of the eye can be disposed at the predetermined depth within the eye. More specifically, while the injector is described above as moving the needle through the eye substantially without expelling the drug formulation in response to the second force, the second force exerted on the portion of the injector (e.g., the handle) can be sufficient to expel the drug formulation through the needle and into the suprachoroidal space when the needle is disposed at a predetermined depth. For example, in some instances, the density of the sclera and the friction force between the piston and the inner surface of the barrel, collectively, are sufficient to resist a distal movement of the piston in response to the second force. Conversely, once the distal surface of the needle is disposed at a depth within the eye (e.g., at or near the suprachoroidal space), the density of that portion of the eye can be less than the density of the sclera. Thus, the collective force exerted by the friction force and the anatomy of the eye in response to the second force is reduced. In this manner, the second force can become sufficient to move the piston in the distal direction within the barrel to expel the drug formulation into the suprachoroidal space. In some instances, the user exerting the second force on the portion of the injector can feel a loss of resistance and/or the like, which can be an indication that the distal surface of the needle is disposed at a desired depth.

While the method 1000 is described above as including a set of steps, in some instances, the method 1000 can include any number of optional steps and/or pre-procedural or post-procedural steps. For example, in some embodiment, a method of delivering a drug formulation to ocular tissue in a clinical study can be similar to the method 1000 and can include at least some of the following steps:

1. Ensure a study participant's eye remains dilated.

2. Anesthetize study eye (e.g., with topical anesthesia).

3. Wait appropriate amount of time after the placement of anesthesia.

4. Sterilize and prep eye, insert lid speculum and ensure eyelids are fully retracted per standard of care, and measure injection site with calipers.

5. Retrieve the study drug kit.

6. Remove vial of a drug formulation and shake vigorously for 10 seconds before use to ensure a uniform suspension.

7. Remove plastic top from vial and prepare vial using standard aseptic techniques.

8. Prepare an injector. The injector can be any of the injectors shown and described herein, such as the injector 100.
a. Attach the provided drug transfer needle (sterile, disposable, hypodermic needle) to the microinjector.
b. Insert the drug transfer needle into the vial by piercing the septum.
c. Invert the vial, inject the air and withdraw >200 μL the drug formulation by pulling back on the microinjector handle.
d. Withdraw drug transfer needle from the vial.
e. Remove the drug transfer needle from the microinjector handle and attach the microneedle (900 μm). The microneedle can include the hub 160 shown and described above.
f. Prime the injector and ensure enough drug is available to deliver 100 μL of the drug formulation into the SCS.

9. After priming, the drug formulation should be injected without delay to prevent settling of the drug in the syringe.

10. Holding the microinjector, insert the microneedle into the sclera perpendicular to the ocular surface. Target location should be approximately 4-5 mm from the limbus and the superior temporal quadrant is the recommended location for suprachoroidal injections. Ensure the approach is as perpendicular to the sclera as possible. Do not bend or angle the microneedle at any time during the procedure.

11. Once the microneedle is inserted into the sclera, ensure that the hub of the microneedle is in firm contact with the conjunctiva. Firm contact of the microneedle injection system with the conjunctiva will be observed as a slight, localized dimple of the globe around the microneedle hub.

12. Stabilize the microneedle with one hand while applying this constant downward force throughout the injection procedure.

13. Using the other hand (if necessary), advance the injector handle until up to 100 μL of the drug formulation is injected over a 5-10 second period. During this process, ensure that nominal pressure continues to be placed on the needle such that it is in tight contact with the conjunctiva.

14. If there is resistance to flow through the microneedle, remove the microneedle from the eye and examine the eye for any issues. If subject safety is not at risk, investigator may choose to verify patency of the microneedle and use best medical judgment to restart the injection procedure at a new site adjacent to the original injection site or use a longer microneedle length (1100 nm). Ensure there is enough the drug formulation remaining in the microinjector to prime the replacement microneedle and deliver a 100 μl dose. Repeat the microinjector process as stated above in step 9.

15. Maintain light pressure on the microneedle once injection is complete and hold for 5-10 seconds.

16. Obtain cotton swab and remove the microneedle slowly from the eye. Simultaneously cover the injection site with the cotton swab.

17. Hold the swab over the injection site with light pressure for a few seconds to ensure minimal reflux upon removal. Remove cotton swab.

18. Remove the lid speculum.

19. Following the SCS injection, assess eye via indirect ophthalmoscope.

Alternatively, preparing an injector, which can be any of the injectors shown and described herein, suhb as the injector 100 (e.g., step 8 above) can include:
a. Attach the provided vial access device (sterile, disposable) to the vial of study drug by inserting it into the vial while the vial is on a flat surface.
b. Detach the cap from the vial access device.
c. Fully pull back on the plunger handle to draw air into the injector.
d. Attach the microinjector handle (syringe) to the vial access device and inject the air.
e. Invert the vial and withdraw >200 μL of the drug formulation by pulling back on the microinjector handle.
f. Replace the vial access device with the 900 nm needle.
g. Recap the vial access device.
h. Mark the injection site with the needle cap or calipers.
i. Prime the microinjector to remove the excess air.
j. Depress the handle until the plunger reaches the 100 μL marking on the syringe.

Although the medical injectors and methods described herein are shown as including a device including a needle and a reservoir including a medicament, in other embodiments, a medical device or kit can include a simulated medicament injector. In some embodiments, the simulated medicament injector can correspond to an actual medicament injector (e.g., the medical injector 100 described above) and can be used, for example, to train a user in the operation of the corresponding actual medical injector, to perform a “sham” injection as part of a clinical trial protocol, or the like.

A simulated medical injector can simulate the actual medical injector in any number of ways. For example, in some embodiments, the simulated medical injector can have a shape corresponding to a shape of the actual medical injector (e.g., injector 100), a size corresponding to a size of the actual medical injector (e.g., injector 100) and/or a weight corresponding to a weight of the actual medical injector (e.g., injector 100). Moreover, in some embodiments, the simulated medical injector can include components that correspond to the components of the actual medical injector. In this manner, the simulated medical injector can simulate the look, feel and sounds of the actual medical injector. For example, in some embodiments, the simulated medical injector can include external components (e.g., a base, a handle, or the like) that correspond to external components of the actual medical injector. In some embodiments, the simulated medical injector can include internal components (e.g., a plunger) that correspond to internal components of the actual medical injector.

In some embodiments, however, the simulated medical injector can be devoid of a medicament and/or those components that cause the medicament to be delivered (e.g., a microneedle). In this manner, the simulated medical injector can be used to train a user in the use of the actual medical injector without exposing the user to a needle and/or a medicament. Moreover, the simulated medical injector can have features to identify it as a training device to prevent a user from mistakenly believing that the simulated medical injector can be used to deliver a medicament.

In some embodiments, a method of delivering a drug formulation to ocular tissue in a clinical study can be similar to the method 1000 and can include at least some of the following steps:

1. Ensure a study participant's eye remains dilated.

2. Anesthetize study eye (e.g., with topical anesthesia).

3. Wait appropriate amount of time after the placement of anesthesia.

4. Sterilize and prep eye, insert lid speculum and ensure eyelids are fully retracted per standard of care, and measure injection site with calipers.

5. Retrieve the study drug kit.

6. Prepare microinjector. The microinjector can be any of the injectors shown and described herein, such as the injector 100. Moreover, the microinjector can be a simulated microinjector, including a needless hub. This preparation includes:
a. Attach the needleless hub to the microinjector handle

7. Prepare for sham (or training) procedure:
a. Holding the microinjector, press the sham needleless hub into the sclera at the target location.
b. Ensure the approach is as perpendicular to the sclera as possible. Do not angle the microinjector at any time during the procedure.
c. Ensure that the needleless hub is in firm contact with the conjunctiva. Firm contact of the microinjector with the conjunctiva will be observed as a slight, localized dimple of the globe around the needleless hub.

8. Administer sham procedure:
a. Maintain the needle hub against the eye while gently depressing the handle throughout the injection procedure. Perform a sham suprachoroidal injection to the study eye over a 5-10 second period.
b. Maintain the needleless hub against the eye for 5-10 seconds following the sham injection.
c. Obtain cotton swab and remove the needleless hub slowly from the eye. Simultaneously cover the sham site with the cotton swab.
d. Hold the swab over the injection site for a few seconds and then remove cotton swab.

9. Remove lid speculum.

10. Following the SCS injection, assess eye via indirect ophthalmoscope.

The microneedle devices described herein also may be adapted to use the one or more microneedles as a sensor to detect analytes, electrical activity, and optical or other signals. The sensor may include sensors of pressure, temperature, chemicals, and/or electromagnetic fields (e.g., light). Biosensors can be located on or within the microneedle, or inside a device in communication with the body tissue via the microneedle. The microneedle biosensor can be any of the four classes of principal transducers: potentiometric, amperometric, optical, and physiochemical. In one embodiment, a hollow microneedle is filled with a substance, such as a gel, that has a sensing functionality associated with it. In an application for sensing based on binding to a substrate or reaction mediated by an enzyme, the substrate or enzyme can be immobilized in the needle interior. In another embodiment, a wave guide can be incorporated into the microneedle device to direct light to a specific location, or for detection, for example, using means such as a pH dye for color evaluation. Similarly, heat, electricity, light, ultrasound or other energy forms may be precisely transmitted to directly stimulate, damage, or heal a specific tissue or for diagnostic purposes.

The microneedle device for non-surgically delivering drug to the suprachoroidal space of the eye of a human subject, in one embodiment, comprises a hollow microneedle. The device may include an elongated housing for holding the proximal end of the microneedle. The device may further include a means for conducting a drug formulation through the microneedle. For example, the means may be a flexible or rigid conduit in fluid connection with the base or proximal end of the microneedle. The means may also include a pump or other devices for creating a pressure gradient for inducing fluid flow through the device. The conduit may in operable connection with a source of the drug formulation. The source may be any suitable container. In one embodiment, the source may be in the form of a conventional syringe. The source may be a disposable unit dose container.

The transport of drug formulation or biological fluid through a hollow microneedle can be controlled or monitored using, for example, one or more valves, pumps, sensors, actuators, and microprocessors. For instance, in one embodiment the microneedle device may include a micropump, microvalve, and positioner, with a microprocessor programmed to control a pump or valve to control the rate of delivery of a drug formulation through the microneedle and into the ocular tissue. The flow through a microneedle may be driven by diffusion, capillary action, a mechanical pump, electroosmosis, electrophoresis, convection or other driving forces. Devices and microneedle designs can be tailored using known pumps and other devices to utilize these drivers. In one embodiment, the microneedle device may further include an iontophoretic apparatus, similar to that described in U.S. Pat. No. 6,319,240 to Beck, for enhancing the delivery of the drug formulation to the ocular tissue. In another embodiment the microneedle devices can further include a flowmeter or other means to monitor flow through the microneedles and to coordinate use of the pumps and valves.

In some embodiments, the flow of drug formulation or biological fluid can be regulated using various valves or gates known in the art. The valve may be one which can be selectively and repeatedly opened and closed, or it may be a single-use type, such as a fracturable barrier. Other valves or gates used in the microneedle devices can be activated thermally, electrochemically, mechanically, or magnetically to selectively initiate, modulate, or stop the flow of material through the microneedles. In one embodiment, the flow is controlled with a rate-limiting membrane acting as the valve.

In other embodiments, the flow of drug formulation or biological fluid can be regulated by the internal friction of various components, the characteristics of the medicament to be injected (e.g., the viscosity) and/or the characteristics of the desired injection site. For example, as described above, in some embodiments, a drug product can be configured for delivery of a specific formulation to a specific location. In such embodiments, a drug product can include a microinjector (e.g., microinjector 100) and a medicament (e.g., triamcinolone or any other formulations described herein) that is configured to deliver the medicament to a specific target region (e.g., the SCS). In this example, the drug product can be configured such that the flow of the medicament is limited when injection is attempted into a different target region having a higher density (e.g., the sclera). Thus, the drug product is configured to regulate the flow by allowing flow when the injection is attempted into the desired target region.

The microneedle, in one embodiment, is part of an array of two or more microneedles such that the method further includes inserting at least a second microneedle into the sclera without penetrating across the sclera. In one embodiment, where an array of two or more microneedles are inserted into the ocular tissue, the drug formulation of each of the two or more microneedles may be identical to or different from one another, in drug, formulation, volume/quantity of drug formulation, or a combination of these parameters. In one case, different types of drug formulations may be injected via the one or more microneedles. For example, inserting a second hollow microneedle comprising a second drug formulation into the ocular tissue will result in delivery of the second drug formulation into the ocular tissue.

In another embodiment, the device includes an array of two or more microneedles. For example, the device may include an array of from 2 to 1000 (e.g., from 2 to 100 or from 2 to 10) microneedles. In one embodiment, a device includes between 1 and 10 microneedles. An array of microneedles may include a mixture of different microneedles. For instance, an array may include microneedles having various lengths, base portion diameters, tip portion shapes, spacings between microneedles, drug coatings, etc. In embodiments wherein the microneedle device comprises an array of two or more microneedles, the angle at which a single microneedle extends from the base may be independent from the angle at which another microneedle in the array extends from the base.

The SCS drug delivery methods provided herein allow for the delivery of drug formulation over a larger tissue area and to more difficult to target tissue in a single administration as compared to previously known needle devices. Not wishing to be bound by theory, it is believed that upon entering the SCS the drug formulation flows circumferentially from the insertion site toward the retinochoroidal tissue, macula, and optic nerve in the posterior segment of the eye as well as anteriorly toward the uvea and ciliary body. In addition, a portion of the infused drug formulation may remain in the SCS as a depot, or remain in tissue overlying the SCS, for example the sclera, near the microneedle insertion site, serving as additional depot of the drug formulation that subsequently can diffuse into the SCS and into other adjacent posterior tissues.

The human subject treated with the methods and devices provided herein may be an adult or a child. In one embodiment, the patient presents with a retinal thickness of greater than 300 μm (e.g., central subfield thickness as measured by optical coherence tomography). In another embodiment, the patient in need of treatment has a BCVA score of ≧20 letters read in each eye (e.g., 20/400 Snellen approximate). In yet another embodiment, the patient in need of treatment has a BCVA score of ≧20 letters read in each eye (e.g., 20/400 Snellen approximate), but ≦70 letters read in the eye in need of treatment.

The patient in one embodiment has macular edema (ME) that involves the fovea. In one embodiment, in a method for treating ME associated with uveitis, the ME is due to the uveits and not due to any other cause. In an embodiment for treating ME following RVO, the ME is due to RVO and not due to any other cause of ME. In a further embodiment, the RVO is branch retinal vein occlusion (BRVO), hemiretinal vein occlusion (HRVO) or central retinal vein occlusion (CRVO). In one embodiment, the patient in need of treatment experiences a decrease in visual acuity due to the ME.

The microneedle devices and non-surgical methods described herein may be used to deliver drug formulations to the eye of a human subject, particularly for the treatment, diagnosis, or prevention of a posterior ocular disorder, such as uveitis (e.g., non-infectious, infectious, intermediate, posterior or pan uveitis), macular edema associated with uveitis, e.g., non-infectious, intermediate, posterior or pan uveitis and macular edema associated with RVO. In one embodiment, the drug formulation comprises an effective amount of an anti-inflammatory drug. In one embodiment, the patient is in need of treatment of macular edema associated with uveitis or macular edema associated with RVO and the drug formulation comprises an anti-inflammatory drug selected from a steroid compound and a non-steroidal anti-inflammatory drug (NSAID). In even a further embodiment, the drug formulation is a triamcinolone formulation, e.g., a triamcinolone acetonide formulation.

Posterior ocular disorders amenable for treatment by the methods, devices and drug formulations described herein can include, but are not limited to, uveitis (e.g., infectious uveitis, non-infectious uveitis, chronic uveitis, and/or acute uveitis), macular edema, diabetic macular edema (DME), macular edema associated with uveitis (encompassing macular edema associated with infectious uveitis and macular edema associated with non-infectious uveitis), macular edema following retinal vein occlusion (RVO), macular edema associated associated with RVO. In some embodiments, the posterior ocular disorder is macular edema associated with uveitis. In a further embodiment, the uveitis is a non-infectious uveitis.

The uveitis can be either acute or chronic uveitis. Uveitis, and macular edema associated with uveitis can be caused by infectious causes leading to infectious uveitis, such as infection with viruses, fungi, parasites, and/or the like. Uveitis can also be caused by non-infectious causes, such as the presence of noninfectious foreign substances in the eye, autoimmune diseases, surgical and/or traumatic injury, and/or the like. Disorders caused by pathogenic organisms that can lead to infectious uveitis, and to macular edema associated with infectious uveitis, include, but are not limited to, toxoplasmosis, toxocariasis, histoplasmosis, herpes simplex or herpes zoster infection, tuberculosis, syphilis, sarcoidosis, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, or iridocyclitis. Acute uveitis and/or macular edema associated with acute uveitis occurs suddenly and may last for up to about six weeks. In chronic uveitis and/or macular edema associated with chronic uveitis, the onset of signs and/or symptoms is gradual, and symptoms last longer than about six weeks.

Signs of uveitis include ciliary injection, aqueous flare, the accumulation of cells visible on ophthalmic examination, such as aqueous cells, retrolental cells, and vitreouscells, keratic precipitates, and hypema. Symptoms of uveitis include pain (such as ciliary spasm), redness, photophobia, increased lacrimation, and decreased vision. Posterior uveitis affects the posterior or choroid part of the eye. Inflammation of the choroid part of the eye is also often referred to as choroiditis. Posterior uveitis is may also be associated with inflammation that occurs in the retina (retinitis) or in the blood vessels in the posterior segment of the eye (vasculitis). In one embodiment, the methods provided herein comprise non-surgically administering to a uveitis patient suffering from macular edema associated with uveitis (e.g., non-infectious uveitis) in need thereof, an effective amount of an anti-inflammatory drug formulation to the SCS of the eye of the patient. In a further embodiment, the patient experiences a reduction in the severity of the symptoms of with macular edema associated with uveitis, after administration of the drug formulation. In one embodiment, the drug is a steroidal compound. In even a further embodiment, the drug is triamcinolone.

In one embodiment, the patient undergoing one of the treatment methods provided herein, for example, the treatment of macular edema associated with uveitis or macular edema associated with RVO, experiences a reduction in fluid accumulation, inflammation, neuroprotection, complement inhibition, drusen formation, scar formation, and/or a reduction in choriocapillaris or choroidal neocasvularization.

Without wishing to be bound by theory, upon non-surgical SCS administration, the drug remains localized in the posterior segment of the eye, specifically, the choroid and retina. Limiting drug exposure to other eye tissues, in one embodiment, reduces the incidences of side effects associated with the prior art methods.

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed.

Treatment regimens will vary based on the therapeutic formulation being delivered and/or the indication being treated. In one embodiment, a single dosing session is effective in treating one of the indications described herein. However, in another embodiment, multiple dosing sessions are employed. In one embodiment, where multiple dosing sessions are employed, the dosing sessions are spaced apart by from about 10 days to about 70 days, or from about 10 days to about 60 days, or from about 10 days to about 50 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In even another embodiment, the multiple dosing sessions are weekly (about every 7 days), bi-weekly (e.g., about every 14 days), about every 21 days, monthly (e.g., about every 30 days), or bi-monthly (e.g., about every 60 days). In yet another embodiment, the dosing sessions are monthly dosing sessions (e.g., from about 28 days to about 31 days) and at least three dosing sessions are employed.

In one embodiment, the non-surgical SCS delivery methods, for example, with one of the devices provided herein, are used to treat a patient in need of treatment of macular edema associated with uveitis (e.g., non-infectious uveitis). In one embodiment, SCS administration of a drug (e.g., an anti-inflammatory compound such as a steroid or NSAID) via the methods described herein reduces the vitreous haze experienced by the patient.

In one embodiment, vitreous haze will is assessed via indirect ophthalmoscopy using a standardized photographic scale ranging from 0 to 4, with 0-4 defined below in Table 1 (Nussenblatt 1985 as modified in Lowder 2011, incorporated by reference herein in their entireties). Vitreous haze in another embodiment, is graded from color fundus photographs according to a similar scale.

TABLE 1

| Score | Description |
|---|---|
| 0 | No inflammation |
| +0.5 | Trace inflammation (slight blurring of the optic disc margins |
| | and/or loss of the nerve fiber layer reflex) |
| +1 | Mild blurring of the retinal vessels and optic nerve |
| +1.5 | Optic nerve head and posterior retina view obsuration greater |
| | than +1 but less than +2 |
| +2 | Moderate blurring of the optic nerve head |
| +3 | Marked blurring of the optic nerve head |
| +4 | Optic nerve head not visible |

In yet another embodiment, the non-surgical SCS delivery methods provided herein reduce the macular edema experienced by a patient suffering from macular edema associated with RVO.

In one embodiment, a method is provided for treating a human patient for macular edema associated with non-infectious uveitis or macular edema associated with RVO. The method comprises non-surgically administering an anti-inflammatory drug (e.g., a steroidal compound such as triamcinolone acetonide) to the SCS of one or both of the patient's eyes, wherein upon administration, the drug is substantially retained in the SCS and/or another posterior region of the eye. In a further embodiment, upon administration, the drug is substantially localized to one or more of the SCS, choroid and/or retina. The efficacy of the method, in one embodiment, is measured by measuring the patient's mean change from baseline in macula thickness at one or more time points after the patient is treated. For example, at one week, two weeks, three weeks, one month, two months, three months, four months or more, including all durations in between, after treatment, e.g., with an anti-inflammatory drug delivered non-surgically to the SCS, mean change from baseline in retinal thickness and/or macula thickness is measured. In a further embodiment, the patient is in need of treatment of macular edema associated with RVO, and a second drug formulation comprising a VEGF modulator (e.g., a VEGF antagonist) is administered to the eye of the patient via an intravitreal injection. In a further embodiment, the VEGF modulator is ranibizumab, aflibercept or bevacizumab.

A decrease in retina thickness and/or macula thickness is one measurement of treatment efficacy of the methods provided herein. For example, in one embodiment, a macular edema associated with uveitis (e.g., non-infectious uveitis) patient or a macular edema associated with RVO patient treated by one of the methods provided herein for example with one of the devices described herein experiences a decrease in retinal thickness from baseline (e.g., retinal thickness such as central subfield thickness (CST) prior to treatment), at any given time point after at least one dosing session (single session or multiple dosing sessions, of at least about 20 μm, or at least about 40 μm, or at least about 50 μm, or at least about 100 μm, or at least about 150 μm or at least about 200 μm, or from about 50-100 μm, and all values in between. In another embodiment, the patient experiences a≧5%, ≧10%, ≧15%, ≧20%, ≧25% decrease in retinal thickness (e.g., CST) subsequent to at least one dosing session.

In one embodiment, the decrease in retinal thickness is measured about 2 weeks, about 1 month, about 2 months, about 3 months or about 6 months after the at least one dosing session. In another embodiment, the decrease in retinal thickness is measured at least about 2 weeks, at least about 1 month, at least about 2 months, at least about 3 months or at least about 6 months after the at least one dosing session. In one embodiment, where multiple dosing sessions are employed, a decrease in retinal thickness is sustained by the patient for at least about 2 weeks, at least about 1 month, at least about 2 months, at least about 3 months or at least about 6 months after each dosing session.

In one embodiment, a macular edema associated with uveitis (e.g., non-infectious uveitis) patient treated by the methods provided herein experiences a decrease in retinal thickness from baseline (i.e., retinal thickness prior to treatment), at any given time point, of from about 20 μm to about 200 μm, at from about 40 μm to about 200 μm, of from about 50 μm to about 200 μm, of from about 100 μm to about 200 μm, or from about about 150 μm to about 200 μm. In one embodiment, change in retinal thickness from baseline is measured as a change in CST, for example, by spectral domain optical coherence tomography (SD-OCT). In one embodiment, the change in retinal thickness

In yet another embodiment, the therapeutic response is a change from baseline in macula thickness at one or more time points after the patient is treated. For example, at one week, two weeks, three weeks, one month, two months, three months, four months or more, including all durations in between, after a dosing session, e.g., with an anti-inflammatory drug such as triamcinolone delivered non-surgically to the SCS, change from baseline in macula thickness is measured. A decrease in macula thickness (as compared to prior to treatment) is one measurement of therapeutic response (e.g., by about 10%, or about 20%, or about 30%, or about 40%, or about 50%, or about 60% and more, including all values in between).

Efficacy, in another embodiment, is assessed via a visual acuity measurement at one and/or two months post treatment (e.g., by measuring the mean change in best corrected visual acuity (BCVA) from baseline, i.e., prior to treatment). In one embodiment, a patient treated by one or more of the methods provided herein experiences an improvement in BCVA from baseline, at any given time point (e.g., 2 weeks after administration, 4 weeks after administration, 2 months after at least one dosing session, 3 months after administration), of at least 2 letters, at least 3 letters, at least 5 letters, at least 8 letters, at least 12 letters, at least 13 letters, at least 15 letters, at least 20 letters, and all values in between, as compared to the patient's BVCA prior to the at least one dosing session.

In one embodiment, the patient, for example a macular edema associated with uveitis patient or a macular edema associated with RVO patient gains about 5 letters or more, about 10 letters or more, 15 letters or more, about 20 letters or more, about 25 letters or more in a BCVA measurement after a dosing regimen is complete, for example a monthly dosing regimen, compared to the patient's BCVA measurement prior to undergoing treatment. In even a further embodiment, the patient gains from about 5 to about 30 letters, 10 to about 30 letters, from about 15 letters to about 25 letters or from about 15 letters to about 20 letters in a BCVA measurement upon completion of at least one dosing session, compared to the patient's BCVA measurement prior to the at least one dosing session. In one embodiment, the BCVA gain is about 2 weeks, about 1 month, about 2 months, about 3 months or about 6 months after the at least one dosing session. In another embodiment, the BCVA is measured at least about 2 weeks, at least about 1 month, at least about 2 months, at least about 3 months or at least about 6 months after the at least one dosing session.

In one embodiment, the BCVA is based on the Early Treatment of Diabetic Retinopathy Study (ETDRS) visual acuity charts and is assessed at a starting distance of 4 meters.

In another embodiment, the patient subjected to a treatment method, e.g., with one of the devices provided herein substantially maintains his or her vision subsequent to the treatment (e.g., a single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

Decrease in vitreous haze can also be used as a measure of the method's efficacy. Decreases in vitreous haze can be qualitatively and/or quantitatively determined by techniques such as, but not limited to, photographic grading, a scoring system, a multi-point scale, a multi-step scale (e.g. a multi-step logarithmic scale, manual screening by one or more examiners, and/or the like).

In one embodiment, the decrease in vitreous haze is present about 2 weeks, about 1 month, about 2 months, about 3 months or about 6 months after the at least one dosing session. In another embodiment, the decrease in retinal thickness is present at least about 2 weeks, at least about 1 month, at least about 2 months, at least about 3 months or at least about 6 months after the at least one dosing session. In one embodiment, where multiple dosing sessions are employed, a decrease in vitreous haze is experienced by the patient and is present at least about 2 weeks, at least about 1 month, at least about 2 months, at least about 3 months or at least about 6 months after each dosing session.

In one embodiment, the methods provided herein provide for effective treatment of a patient who had previously undergone treatment for uveitis (e.g., non-infectious uveitis), macular edema associated with uveitis (e.g., non-infectious uveitis) or macular edema associated with RVO, but was unresponsive, or not properly responsive to the prior treatment for the respective posterior ocular disorder. For example, in one embodiment, a patient undergoing a treatment method for uveitis (e.g., non-infectious uveitis), macular edema associated with uveitis (e.g., non-infectious uveitis) or macular edema associated with RVO of the present invention was previously treated for macular edema associated with uveitis (e.g., non-infectious uveitis), or macular edema associated with RVO, but was unresponsive or not properly responsive. As one of skill in the art will appreciate, a patient unresponsive or not properly responsive to treatment does not exhibit an improvement in a symptom or improvement in a clinical manifestation of macular edema associated with uveitis (e.g., non-infectious uveitis), macular edema associated with RVO or wet AMD. In one embodiment, the symptom or clinical manifestation is lesion size, inflammation, edema, visual acuity and/or vitreous haze.

In patients undergoing ocular treatment via shunts or cannulae, or other surgical methods, a marked increase or decrease in intraocular pressure has been reported after the treatment method commences. In one embodiment, the intraocular pressure (TOP) of the patient's eye undergoing treatment for macular edema associated with uveitis (e.g., non-infectious uveitis), macular edema associated with RVO, 2 minutes, 10 minutes, 15 minutes, 30 minutes or 1 hour after suprachoroidal drug administration according to the devices (e.g., the device 100) and/or the methods disclosed herein, is substantially the same IOP, compared to the IOP of the patient's eye prior to administration of the drug for treating macular edema associated with uveitis. In one embodiment, the IOP of the patient's eye undergoing treatment for macular edema associated with uveitis (e.g., non-infectious uveitis), macular edema associated with RVO or wet AMD, 2 minutes, 10 minutes, 15 minutes, 30 minutes or 1 hour after suprachoroidal drug administration, varies by no more than 10%, compared to the IOP of the patient's eye prior to administration of the drug for treating macular edema associated with uveitis (e.g., non-infectious uveitis), macular edema associated with RVO or wet AMD. In one embodiment, the IOP of the patient's eye undergoing treatment for the macular edema associated with uveitis (e.g., non-infectious uveitis), macular edema associated with RVO or wet AMD, 2 minutes, 10 minutes, 15 minutes or 30 minutes after suprachoroidal drug administration, varies by no more than 20%, compared to the IOP of the patient's eye prior to administration of the drug for treating macular edema associated with uveitis (e.g., non-infectious uveitis), macular edema associated with RVO. In one embodiment, the IOP of the patient's eye undergoing treatment for macular edema associated with uveitis (e.g., non-infectious uveitis), macular edema associated with RVO or wet AMD, 2 minutes, 10 minutes, 15 minutes or 30 minutes after suprachoroidal drug administration, varies by no more than 10%-30%, compared to the IOP of the patient's eye prior to administration of the drug for treating macular edema associated with uveitis (e.g., non-infectious uveitis), macular edema associated with RVO or wet AMD. In a further embodiment, the effective amount of the drug for treating macular edema associated with uveitis (e.g., non-infectious uveitis), macular edema associated with RVO or wet AMD comprises an effective amount of an anti-inflammatory drug (e.g., triamcinolone).

In one aspect, the methods described herein relate to the non-surgical administration of a drug formulation for the treatment of uveitis (infectious or non-infectious), macular edema, macular edema associated with non-infectious uveitis, macular edema associated with infectious uveitis, macular edema associated with RVO, wherein the majority of the drug formulation is retained in the SCS and/or other posterior ocular tissue, in one or both eyes of a patient in need of treatment of the posterior ocular disorder, for a period of time after the non-surgical treatment method is completed. Without wishing to be bound by theory, drug formulation retention in the SCS contributes to the sustained release profile of the drug formulations described herein. As described herein, in some embodiments where a patient is treated for macular edema associated with RVO, the patient is further administered a VEGF modulator intravitreally in addition to a non-surgical suprachoroidal injection of an anti-inflammatory compound (e.g., a steroid such as triamcinolone).

The method of treating uveitis (e.g., non-infectious uveitis), macular edema associated with uveitis, macular edema associated with RVO, or wet AMD in a human subject in need thereof comprises, in one embodiment, non-surgically administering a drug formulation to the suprachoroidal space of the affected eye of the human subject, wherein upon administration, the drug formulation flows away from the insertion site and is substantially localized to the posterior segment of the eye, for example to the posterior ocular tissue such as the retina and/or choroid. In one embodiment, the non-surgical methods provided herein allow for longer retention of the drug in the eye, e.g., the posterior segment of the eye, as compared to intravitreal, topical, parenteral, intracameral or oral administration of the same drug dose.

In one embodiment, the suprachoroidal drug dose sufficient to achieve a therapeutic response in a human subject treated with the non-surgical SCS drug delivery method is less than the intravitreal, parenteral, intracameral, topical, or oral drug dose sufficient to elicit the identical or substantially identical therapeutic response. In a further embodiment, the suprachoroidal drug dose is at least 10 percent less than the oral, parenteral or intravitreal dose sufficient to achieve the identical or substantially identical therapeutic response. In a further embodiment, the suprachoroidal dose is about 10 percent to about 25 percent less, or about 10 percent to about 50 percent less than the oral, parenteral, intracameral, topical, or intravitreal dose sufficient to achieve the identical or substantially identical therapeutic response. Accordingly, in one embodiment, the non-surgical SCS administration method of treating macular edema associated with uveitis, macular edema associated with RVO achieves a greater therapeutic efficacy than other routes of administration. In one embodiment, the non-surgical method provided herein comprises inserting a hollow microneedle into the sclera of the eye of the human subject and infusing a drug formulation through the hollow microneedle and into the suprachoroidal space of the eye. As described in more detail below, the drug formulation, in one embodiment, is a solution or suspension of the drug.

In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space.

The amount of drug delivered within the SCS also may be controlled, in part, by the type of microneedle used and how it is used. In one embodiment, a hollow microneedle is inserted into the ocular tissue and progressively retracted from the ocular tissue after insertion to deliver a fluid drug, where after achieving a certain dosage, the delivery could be stopped by deactivating the fluid driving force, such as pressure (e.g., from a mechanical device such as a syringe) or an electric field, to avoid leakage/uncontrolled deliver of drug. Desirably, the amount of drug being delivered is controlled by driving the fluid drug formulation at a suitable infusion pressure. In one embodiment, the infusion pressure may be at least 150 kPa, at least 250 kPa, or at least 300 kPa. In another embodiment, the infusion pressure is about 150 kPa to about 300 kPa. Suitable infusion pressures may vary with the particular patient or species. In another embodiment, the methods provided herein are carried out with one of the devices described above (e.g, the injector 100) or in PCT/US2014/36590, filed May 2, 2014 and entitled “Apparatus and Method for Ocular Injection,” incorporated by reference herein in its entirety for all purposes.

It should be noted that the desired infusion pressure to deliver a suitable amount of drug formulation might be influenced by the depth of insertion of the microneedle and the composition of the drug formulation. For example, a greater infusion pressure may be required in embodiments wherein the drug formulation for delivery into the eye is in the form of or includes nanoparticles or microparticles encapsulating the active agent or microbubbles. Nanoparticle or microparticle encapsulation techniques are well known in the art. In one embodiment, the drug formulation is comprised of drug particles in suspension with a D₉₉ of 10 μm or less. In one embodiment, the drug formulation is comprised of drug particles in suspension with a D₉₉ of 7 μm or less. In another embodiment, the drug formulation is comprised of drug particles in suspension with a D₉₉ of 3 μm or less. In another embodiment, the drug formulation is comprised of drug particles in suspension with a D₅₀ of 5 μm or less. In one embodiment, the drug formulation is comprised of drug particles in suspension with a D₅₀ 1 μm or less.

In one embodiment, the non-surgical method of administering a drug to the SCS further includes partially retracting the hollow microneedle after insertion of the microneedle into the eye, and before and/or during the infusion of the drug formulation into the suprachoroidal space. In a particular embodiment, the partial retraction of the microneedle occurs prior to the step of infusing the drug formulation into the ocular tissue. This insertion/retraction step may form a pocket and beneficially permits the drug formulation to flow out of the microneedle unimpeded or less impeded by ocular tissue at the opening at the tip portion of the microneedle. This pocket may be filled with drug formulation, but also serves as a conduit through with drug formulation can flow from the microneedle, through the pocket and into the suprachoroidal space.

In one embodiment, the methods provided herein, for example, the methods for treating macular edema associated with uveitis, allow for greater drug retention in the eye compared to other drug delivery methods, for example, a greater amount of drug is retained in the eye when delivered via the methods provided herein as compared to the same dose delivered via intracameral, sub-tenon, intravitreal, topical, parenteral or oral drug delivery methods. Accordingly, in one embodiment, the intraocular elimination half life (t_{1/2}) of the drug when delivered via the methods described herein is greater than the intraocular t_{1/2} of the drug when the same drug dose is administered intravitreally, intracamerally, topically, parenterally or orally. In another embodiment, the intraocular Cmax of the drug, when delivered via the methods described herein, is greater than the intraocular Cmax of the drug when the same drug dose is administered intravitreally, intracamerally, sub-tenonally, topically, parenterally or orally. In another embodiment, the mean intraocular area under the curve (AUCO-t) of the drug, when administered to the SCS via the methods described herein, is greater than the intraocular AUCO-t of the drug, when administered intravitreally, intracamerally, sub-tenonally, topically, parenterally or orally. In yet another embodiment, the intraocular time to peak concentration (tmax) of the drug, when administered to the SCS via the methods described herein, is greater than the intraocular tmax of the drug, when the same drug dose is administered intravitreally, intracamerally, topically, parenterally or orally. In a further embodiment, the drug is an an anti-inflammatory drug (e.g., a steroid or NSAID).

In one embodiment, the intraocular t_{1/2} of the drug when administered via the non-surgical SCS drug delivery methods provided herein, is longer than the intraocular t_{1/2} of the drug when the identical dose is administered topically, intracamerally, intravitreally, orally or parenterally. In a further embodiment, the intraocular t_{1/2} of the drug when administered via the non-surgical SCS drug delivery methods provided herein, is from about 1.1 times to about 10 times longer, or from about 1.25 times to about 10 times longer, or from about 1.5 times to about 10 times longer, or about 2 times to about 5 times longer, than the intraocular t1/2 of the drug when the identical dosage is administered topically, intracamerally, sub-tenonally, intravitreally, orally or parenterally. In a further embodiment, the drug is an an anti-inflammatory drug (e.g., a steroid or NSAID).

In another embodiment, the intraocular Cmax of the drug, when delivered via the methods described herein, is greater than the intraocular Cmax of the drug when the same drug dose is administered intravitreally, intracamerally, topically, parenterally or orally. In a further embodiment, the intraocular Cmax of the drug when administered via the non-surgical SCS drug delivery methods provided herein, is at least 1.1 times greater, or at least 1.25 times greater, or at least 1.5 times greater, or at least 2 times greater, or at least 5 times greater, than the intraocular Cmax of the drug when the identical dose is administered topically, intracamerally, intravitreally, orally or parenterally. In one embodiment, the intraocular Cmax of the drug when administered via the non-surgical SCS drug delivery methods provided herein, is about 1 to about 2 times greater, or about 1.25 to about 2 times greater, or about 1 to about 5 times greater, or about 1 to about 10 times greater, or about 2 to about 5 times greater, or about 2 to about 10 times greater, than the intraocular Cmax of the drug when the identical dose is administered topically, intracamerally, sub-tenonally, intravitreally, orally or parenterally. In a further embodiment, the drug is an anti-inflammatory drug (e.g., a steroid or NSAID). In one embodiment, the drug is triamcinolone, infliximab, mycophenolate, methotrexate, sorafenib, axitinib or nepafenac.

In another embodiment, the mean intraocular area under the curve (AUC₀₋ₜ) of the drug, when administered to the SCS via the methods described herein, is greater than the intraocular AUC₀₋ₜ of the drug, when administered intravitreally, intracamerally, sub-tenonally, topically, parenterally or orally. In a further embodiment, the intraocular AUCₒ₋ₜ of the drug when administered via the non-surgical SCS drug delivery methods provided herein, is at least 1.1 times greater, or at least 1.25 times greater, or at least 1.5 times greater, or at least 2 times greater, or at least 5 times greater, than the intraocular AUC0-t of the drug when the identical dose is administered topically, intracamerally, sub-tenonally, intravitreally, orally or parenterally. In one embodiment, the intraocular AUC₀₋ₜ of the drug when administered via the non-surgical SCS drug delivery methods provided herein, is about 1 to about 2 times greater, or about 1.25 to about 2 times greater, or about 1 to about 5 times greater, or about 1 to about 10 times greater, or about 2 to about 5 times greater, or about 2 to about 10 times greater, than the intraocular AUCO-t of the drug when the identical dose is administered topically, intracamerally, sub-tenonally, intravitreally, orally or parenterally. In a further embodiment, the drug is an anti-inflammatory drug (e.g., a steroid or NSAID).

In one embodiment, the drug formulation comprising the effective amount of the drug (e.g., an anti-inflammatory drug (e.g., a steroid such as triamcinolone or NSAID), once delivered to the SCS, is substantially retained in the SCS over a period of time. For example, in one embodiment, about 80% of the drug formulation is retained in the SCS for about 30 minutes, or about 1 hour, or about 4 hours or about 24 hours or about 48 hours or about 72 hours. In this regard, a depot of drug is formed in the SCS and/or surrounding tissue, to allow for sustained release of the drug over a period of time.

In one embodiment, the non-surgical suprachoroidal drug delivery methods provided herein result in an increased therapeutic efficacy and/or improved therapeutic response, as compared to oral, parenteral, sub-tenon, and/or intravitreal drug delivery methods of the identical or similar drug dose, for treatment of uveitis (e.g., non-infectious uveitis), macular edema associated with uveitis (e.g., non-infectious uveitis) or macular edema associated with RVO. In one embodiment, the SCS drug dose sufficient to provide a therapeutic response is about 90%, or about 75%, or about one-half (e.g., about one half or less) the intravitreal, intracameral, topical, oral or parenteral drug dose sufficient to provide the same or substantially the same therapeutic response. In another embodiment, the SCS dose sufficient to provide a therapeutic response is about one-fourth the intravitreal, intracameral, sub-tenon, topical, oral or parenteral drug dose sufficient to provide the same or substantially the same therapeutic response. In yet another embodiment, the SCS dose sufficient to provide a therapeutic response is one-tenth the intravitreal, intracameral, sub-tenon, topical, oral or parenteral drug dose sufficient to provide the same or substantially the same therapeutic response. In one embodiment, the therapeutic response is a decrease in inflammation, as measured by methods known to those of skill in the art. In another embodiment, the therapeutic response is a decrease in number of ocular lesions, or decrease in ocular lesion size. In another embodiment, the therapeutic response is a decrease in fluid accumulation and/or intraocular pressure.

Therapeutic response is measured at a time point post-treatment, for example 5 days, 1 week, 2 weeks, 3 weeks, 4 weeks, 5 weeks, 6 weeks, 7 weeks, 8 weeks, 9 weeks, 10 weeks, 11 weeks or 12 weeks post-treatment, and all values in between.

The therapeutic efficacy of the drug formulations delivered by the methods described herein and therapeutic response of the human subject can be assayed by standard means in the art, as known to those of skill in the art. In general, the therapeutic efficacy of any particular drug can be assessed by measuring the response of the human subject after administration of the drug; a drug with a high therapeutic efficacy will show a greater amelioration and/or discontinuation of symptoms than a drug with a lower therapeutic efficacy. In non-limiting examples, the efficacy of the drug formulations (e.g., an angiogenesis inhibitor, an anti-inflammatory drug (e.g., a steroid or NSAID), a VEGF modulator (e.g., a VEGF antagonist), a PDGF modulator (e.g., a PDGF antagonist), a compound with both VEGF and PDGF antagonist activity, or a vascular permeability inhibitor formulation) provided herein can be measured, for example, by observing changes in pain intensity, changes in ocular lesions (size or number), intraocular pressure, fluid accumulation, inflammation (e.g., by measuring changes in the Hackett/McDonald ocular score), ocular hypertension, and/or visual acuity.

In another embodiment, the efficacy of the therapeutic formulation is measured by observing changes in the measurements according to the Hackett/McDonald ocular scores, inflammation, visual acuity, and/or edema. In another embodiment, the efficacy of the therapeutic formulation is measured, for example, by observing changes in the measurements according to the Hackett/McDonald ocular scores, inflammation, visual acuity, and/or edema.

In one embodiment, the non-surgical administration of an effective amount of a drug formulation to the SCS results to treat uveitis (e.g., non-infectious uveitis), macular edema associated with uveitis, macular edema associated with RVO or wet AMD results in a decreased number of deleterious side effects or clinical manifestations in the treated patient as compared to the number of side effects or clinical manifestations caused by the same drug dose administered intravitreally, intracamerally, orally or parenterally. In another embodiment, the non-surgical administration of an effective amount of a drug formulation to the SCS results in a decreased number of one or more deleterious side effects or clinical manifestations, as compared to the deleterious side effects or clinical manifestations caused by the same drug dose administered intravitreally, intracamerally, sub-tenonally, orally or parenterally.

Examples of side effects and clinical manifestations that can be reduced or ameliorated include, but are not limited to, inflammation, gastrointestinal side effects (e.g., diarrhea, nausea, gastroenteritis, vomiting, gastrointestinal, rectal, and duodenal hemorrhage, hemorrhagic pancreatitis, large intestine perforation black or bloody stools, and/or coughing up blood); hematologic side effects (e.g., leucopenia, anemia, pancytopenia and agranulocytosis, thrombocytopenia, neutropenia, pure red cell aplasia (PRCA), deep venous thrombosis easy bruising, and/or unusual bleeding from the nose, mouth, vagina, or rectum); immunologic side effects/clinical manifestations (e.g., immunosuppression, immunosuppression resulting in sepsis, opportunistic infections (herpes simplex virus, herpes zoster, and invasive candidal infections), and/or increased infection); oncologic side effects/clinical manifestations (e.g., lymphoma, lymphoproliferative disease and/or non-melanoma skin carcinoma); renal side effects/clinical manifestations (e.g. dysuria, urgency, urinary tract infections, hematuria, kidney tubular necrosis, and/or BK virus-associated nephropathy); metabolic side effects/clinical manifestations (e.g. edema, hyperphosphatemia, hypokalemia, hyperglycemia, hyperkalemia, swelling, rapid weight gain, and/or enlarged thyroid); respiratory side effects/clinical manifestations (e.g., respiratory infection, dyspnea, increased cough, primary tuberculosis dry cough, wheezing, and/or stuffy nose); dermatologic side effects/clinical manifestations (e.g., acne, rash, dyshidrotic eczema, papulosquamous psoriatic-like skin eruption rash, blisters, oozing, mouth sores, and/or hair loss); muscoskeletal side effects/clinical manifestations (e.g. myopathy and/or muscle pain), hepatic side effects/clinical manifestations (e.g. hepatoxicity and/or jaundice), abdominal pain, increased incidence of first trimester pregnancy loss, missed menstrual periods, severe headache, confusion, change in mental status, vision loss, seizure (convulsions), increased sensitivity to light, dry eye, red eye, itchy eye, and/or high blood pressure. As provided above, the reduction or amelioration of the side effect or clinical manifestation is a reduction or amelioration, as compared to the severity of the side effect or clinical manifestation prior to administration of the drug formulation to the SCS of the eye of the patient, or a reduction or amelioration of the side effect or clinical manifestation in the patient, as compared to the reduction or amelioration experienced upon intravitreal, intracameral, parenteral or oral administration of the same drug.

A wide range of therapeutic formulations, for example those that include one or more drugs and/or cellular therapies may be formulated for delivery to the suprachoroidal space and posterior ocular tissues with the present microneedle devices and methods. As used herein, the term “drug” refers to any prophylactic, therapeutic, or diagnostic agent, i.e., an ingredient useful for medical applications. The drug may be selected from cellular therapeutics, small molecules, biologics such as proteins, peptides and fragments thereof, nucleic acids including vectors encoding nucleic acid gene therapeutics, which can be naturally occurring, synthesized or recombinantly produced. For example, in one embodiment, the drug delivered to the suprachoroidal space with the non-surgical methods described herein is an antibody or a fragment thereof (e.g., a Fab, Fv or Fc fragment). In certain embodiments, the drug is a sub-immunoglobulin antigen-binding molecule, such as Fv immunoglobulin fragment, minibody, diabody, and the like, as described in U.S. Pat. No. 6,773,916, incorporated herein by reference in its entirety for all purposes. In one embodiment, the drug is a humanized antibody or a fragment thereof.

In one embodiment, the non-surgical treatment methods and devices described herein may be used in gene-based therapy applications. For example, the method, in one embodiment, comprises administering a drug formulation into the suprachoroidal space to deliver select DNA, RNA, or oligonucleotides to targeted ocular tissues. Accordingly, in one embodiment, the drug is selected from a suitable oligonucleotide (e.g., antisense oligonucleotide agents), polynucleotide (e.g., therapeutic DNA), ribozyme, dsRNA, siRNA, RNAi, gene therapy vectors, and/or vaccine. In a further embodiment, the drug is an aptamer (e.g., an oligonucleotide or peptide molecule that binds to a specific target molecule).

In one embodiment, a nucleic acid therapeutic is delivered by one of the devices and/or methods provided herein. In a further embodiment, the nucleic acid therapeutic is delivered via a viral particle (viral vector). The virus particle, in one embodiment, is an adenovirus, (Ad), adenoassociated virus (AAV), or lentivirus. In another embodiment, the viral vector is a self-complementary AAV (scAAV) or helper-dependent adenovirus (HD-Ad). In another embodiment, a plasmid vector expressing siRNA or other nucleic acid therapeutic is delivered via one of the devices and/or methods described herein. Alternatively or additionally, a nucleic acid therapeutic is delivered via a (1) polymeric, (2) lipid (e.g., liposomal), (3) protein or (4) dendrimeric nanocarrier delivery system.

In another embodiment, the drug formulation delivered via the methods provided herein comprises a small molecule drug, an endogenous protein or fragment thereof, or an endogenous peptide or fragment thereof.

Representative examples of types of drugs for delivery to ocular tissues for the treatment of uveitis (e.g., non-infectious uveitis), macular edema associated with uveitis (e.g., non-infectious uveitis), macular edema associated with RVO and/or wet AMD include anti-inflammatory drugs, including, but not limited to steroids (e.g., triamcinolone), immunosuppressives, antimetabolites, T-cell inhibitors, alkylating agents, biologics, TNF antagonists (e.g., TNF-α antagonists), vascular endothelial growth factor (VEGF) modulators (e.g., VEGF antagonists), and/or non-steroidal anti-inflammatory drugs (NSAIDs). Non-limiting examples of specific drugs and classes of drugs that can be delivered to the suprachoroidal space to treat macular edema associated with uveitis include miotics (e.g., pilocarpine, carbachol, physostigmine), sympathomimetics (e.g., adrenaline, dipivefrine), carbonic anhydrase inhibitors (e.g., acetazolamide, dorzolamide), VEGF antagonists, platelet derived growth factor (PDGF) modulators (e.g., PDGF antagonists), NSAIDs, steroids, prostaglandins, anti-microbial compounds, including anti-bacterials and anti-fungals (e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines), aldose reductase inhibitors, anti-inflammatory and/or anti-allergy compounds (e.g., steroidal compounds such as triamcinolone, betamethasone, clobetasone, dexamethasone, fluorometholone, hydrocortisone, prednisolone and non-steroidal compounds such as antazoline, bromfenac, diclofenac, indomethacin, lodoxamide, saprofen, sodium cromoglycate), artificial tear/dry eye therapies, local anesthetics (e.g., amethocaine, lignocaine, oxbuprocaine, proxymetacaine), cyclosporine, diclofenac, urogastrone and growth factors such as epidermal growth factor, mydriatics and cycloplegics, mitomycin C, and collagenase inhibitors and treatments of age-related macular degeneration such as pegagtanib sodium, ranibizumab, and bevacizumab. In one embodiment, the drug delivered by one of the devices and/or methods described herein is ranibizumab, axitinib, bevacizumab and/or aflibercept.

As provided throughout, in the methods provided herein for the treatment of uveitis (e.g., non-infectious uveitis), macular edema associated with uveitis, and macular edema associated with RVO, a therapeutic formulation comprising an effective amount of an anti-inflammatory drug (e.g., a steroid or NSAID) and/or a VEGF modulator (e.g., a VEGF antagonist) is delivered non-surgically to the SCS of an eye of a patient in need thereof.

In one embodiment, an angiogenesis inhibitor is administered to the SCS of a patient in need thereof. The angiogenesis inhibitor delivered via the methods and devices described herein, in one embodiment, is interferon gamma 1β, interferon gamma 1β (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2β, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260 or XV615.

In one embodiment, the drug delivered to the suprachoroidal space is sirolimus (Rapamycin®, Rapamune®). In one embodiment, the non-surgical drug delivery methods disclosed herein are used in conjunction with rapamycin to treat, prevent and/or ameliorate macular edema associated with uveitis or macular edema associated with RVO. In addition, delivery of rapamycin using the microneedle devices and methods disclosed herein may be combined with one or more agents listed herein or with other agents known in the art. In a further embodiment, the macular edema associated with uveitis is macular edema associated with non-infectious uveitis.

In one embodiment, the drug delivered to the suprachoroidal space using the non-surgical methods (e.g., microneedle devices and methods) herein to treat macular edema associated with uveitis or macular edema associated with RVO is triamcinolone (e.g., triamcinolone acetonide). In one embodiment, the non-surgical drug delivery methods disclosed herein are used in conjunction with triamcinolone to treat, prevent and/or ameliorate macular edema associated with uveitis (e.g., non-infectious uveitis or infectious uveitis). In addition, delivery of rapamycin using the microneedle devices and methods disclosed herein may be combined with one or more agents listed herein or with other agents known in the art. In a further embodiment, the macular edema associated with uveitis is macular edema associated with non-infectious uveitis.

In one embodiment, a VEGF modulator is delivered via one of the devices desvirbed herein. In one embodiment, the VEGF modulator is a VEGF antagonist. In one embodiment, the VEGF modulator is a VEGF-receptor kinase antagonist, an anti-VEGF antibody or fragment thereof, an anti-VEGF receptor antibody, an anti-VEGF aptamer, a small molecule VEGF antagonist, a thiazolidinedione, a quinoline or a designed ankyrin repeat protein (DARPin). As described herein, in some embodiments for the treatment of macular edema associated with RVO, an anti-inflammatory drug is delivered to the SCS of the eye of a patient in need thereof, in combination with intravitreal delivery of a VEGF modulator (e.g., VEGF antagonist) to the same eye. In one embodiment, the VEGF antagonist is an antagonist of a VEGF receptor (VEGFR), i.e., a drug that inhibits, reduces, or modulates the signaling and/or activity of a VEGFR. The VEGFR may be a membrane-bound or soluble VEGFR. In a further embodiment, the VEGFR is VEGFR-1, VEGFR-2 or VEGFR-3. In one embodiment, the VEGF antagonist targets the VEGF-C protein. In another embodiment, the VEGF modulator is an antagonist of a tyrosine kinase or a tyrosine kinase receptor. In another embodiment, the VEGF modulator is a modulator of the VEGF-A protein. In yet another embodiment, the VEGF antagonist is a monoclonal antibody. In a further embodiment, the monoclonal antibody is a humanized monoclonal antibody.

In one embodiment, the VEGF modulator is one or more of the following: AL8326, 2C3 antibody, AT001 antibody, HyBEV, bevacizumab (Avastin®), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, sunitinib malate (Sutent®), INDUS815C, R84 antibody, KD019, NM3, allogenic mesenchymal precursor cells combined with an anti-VEGF antagonist (e.g., anti-VEGF antibody), MGCD265, MG516, VEGF-Receptor kinase inhibitor, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Flt1 receptor, cediranib (Recentin™), AV-951, tivozanib (KRN-951), regorafenib (Stivarga®), volasertib (BI6727), CEP11981, KH903, lenvatinib (E7080), lenvatinib mesylate, terameprocol (EM1421), ranibizumab (Lucentis®), pazopanib hydrochloride (Votrient™), PF00337210, PRS050, SP01 (curcumin), carboxyamidotriazole orotate, hydroxychloroquine, linifanib (ABT869, RG3635), fluocinolone acetonide (Iluvien®), ALG1001, AGN150998, DARPin MP0112, AMG386, ponatinib (AP24534), AVA101, nintedanib (Vargatef™), BMS690514, KH902, golvatinib (E7050), everolimus (Afinitor®), dovitinib lactate (TKI258, CHIR258), ORA101, ORA102, axitinib (Inlyta®, AG013736), plitidepsin (Aplidin®), PTC299, aflibercept (Zaltrap®, Eylea®), pegaptanib sodium (Macugen™, LI900015), verteporfin (Visudyne®), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PTI101, TG100948, ICS283, XL647, enzastaurin hydrochloride (LY317615), BC194, quinolines, COT601M06.1, COT604M06.2, MabionVEGF, SIR-Spheres coupled to anti-VEGF or VEGF-R antibody, Apatinib (YN968D1), and AL3818. In addition, delivery of a VEGF antagonist using the microneedle devices and non-surgical methods disclosed herein may be combined with one or more agents listed herein or with other agents known in the art, either in a single or multiple formulations.

In one embodiment, an immunosuppressive agent is delivered via one of the devices described herein. In a further embodiment, the immunosuppressive agent is a glucocorticoid, cytokine inhibitor, cytostatic, alkylating agent, anti-metabolite, folic acid analogue, cytotoxic antibiotic, interferon, opioid, T-cell receptor directed antibody or an IL-2 receptor directed antibody. In one embodiment, the immunosuppressive agent is an anti-metabolite and the anti-metabolite is a purine analog, pyrimidine analogue, folic acid analogue or a protein synthesis inhibitor. In another embodiment, the immunosuppressive agent is an interleukin-2 inhibitor (e.g., basiliximab or daclizumab). Other immunosuppressive agents amenable for use with the methods and formulations described herein include, but are not limited to cyclophosphamide, nitrosourea, methotrexate, azathioprine, mercaptopurine, fluorouracil, dactinomycin, anthracycline, mitomycin C, bleomycin, mithramycin, muromonab-CD3, cyclosporine, tacrolimus, sirolimus or mycophenolate. In one embodiment, the drug formulation comprises an effective amount mycophenolate.

In one embodiment, the drug formulation delivered to the SCS of an eye of a patient in need thereof via the methods described herein comprises an effective amount of vascular permeability inhibitor. In one embodiment, the vascular permeability inhibitor is a vascular endothelial growth factor (VEGF) antagonist or an angiotensin converting enzyme (ACE) inhibitor. In a further embodiment, the vascular permeability inhibitor is an angiotensin converting enzyme (ACE) inhibitor and the ACE inhibitor is captopril.

In one embodiment, the drug is a steroid or a non-steroid anti-inflammatory drug (NSAID). In another embodiment, the anti-inflammatory drug is an antibody or fragment thereof, an anti-inflammatory peptide(s) or an anti-inflammatory aptamer(s). As provided throughout the specification, the delivery of the anti-inflammatory drug to the suprachoroidal space results in benefits over administration of the same drug delivered via oral, intravitreal, intracameral, topical and/or a parenteral route of administration. For example, in one embodiment, the therapeutic effect of the drug delivered to the suprachoroidal space is greater than the therapeutic effect of the same drug, delivered at the same dosage, when the drug is delivered via oral, intravitreal, topical or parenteral route. In one embodiment, the intraocular elimination half life (t1/2) of the anti-inflammatory drug administered to the SCS is greater than the intraocular t1/2 of the anti-inflammatory drug, when the identical dosage of the anti-inflammatory drug is administered intravitreally, intracamerally, topically, parenterally or orally. In another embodiment, the mean intraocular maximum concentration (Cmax) of the anti-inflammatory drug, when administered to the SCS via the methods described herein, is greater than the intraocular Cmax of the anti-inflammatory drug, when administered intravitreally, intracamerally, topically, parenterally or orally. In another embodiment, the mean intraocular area under the curve (AUC0-t) of the anti-inflammatory drug, when administered to the SCS via the methods described herein, is greater than the intraocular AUC0-t of the anti-inflammatory drug, when the identical dosage of the anti-inflammatory drug is administered intravitreally, intracamerally, topically, parenterally or orally.

Steroidal compounds that can be administered via the methods provided herein include hydrocortisone, hydrocortisone-17-butyrate, hydrocortisone-17-aceponate, hydrocortisone-17-buteprate, cortisone, tixocortol pivalate, prednisolone, methylprednisolone, prednisone, triamcinolone, triamcinolone acetonide, mometasone, amcinonide, budesonide, desonide, fluocinonide, halcinonide, bethamethasone, bethamethasone dipropionate, dexamethasone, fluocortolone, hydrocortisone-17-valerate, halometas one, alclometasone dipropionate, prednicarbate, clob etas one-17-butyrate, clobetasol-17-propionate, fluocortolone caproate, fluocortolone pivalate, fluprednidene acetate and prednicarbate.

Specific classes of NSAIDs that can be administered via the methods provided herein include salicylates, propionic acid derivatives, acetic acid derivatives, enolic acid derivatives, fenamic acid derivatives and cyclooxygenase-2 (COX-2) inhibitors. In one embodiment, the methods provided herein are used to deliver one or more of the following NSAIDs to the SCS of an eye of a patient in need thereof: acetylsalicylic acid, diflunisal, salsalate, ibuprofen, dexibuprofen, naproxen, fenoprofen, keotoprofen, dexketoprofen, flurbiprofen, oxaprozin, loxaprofen, indomethacin, tolmetin, sulindac, etodolac, ketorolac, diclofenac or nabumetone, piroxicam, meloxicam, tenoxicam, droxicam, lornoxicam or isoxicam, mefanamic acid, meclofenamic acid, flufenamic acid, tolfenamic acid, celecoxib, refecoxib, valdecoxib, parecoxib, lumiracoxib, etoricoxib or firocoxib.

Other examples of anti-inflammatory drugs, that can be used in the methods provided herein for treating macular edema associated with uveitis (infectious or non-infectious uveitis) include, but are not limited to: mycophenolate, remicase, nepafenac, 19AV agonist(s), 19GJ agonists, 2MD analogs, 4SC101, 4SC102, 57-57, 5-HT2 receptor antagonist, 64G12, A804598, A967079, AAD2004, AB1010, AB224050, abatacept, etaracizumab (Abegrin™), Abevac®, AbGn134, AbGn168, Abki, ABN912, ABR215062, ABR224050, cyclosporine (Abrammune®), docosanol (behenyl alcohol, Abreva®), ABS15, ABS4, ABS6, ABT122, ABT325, ABT494, ABT874, ABT963, ABXIL8, ABXRB2, AC430, Accenetra, lysozyme chloride (Acdeam®), ACE772, aceclofenac (Acebloc, Acebid, Acenac), acetaminophen, chlorzoxazone, serrapeptase, tizanidine hydrochloride, betadex, Aceclogesic Plus, Aceclon, Acecloren, Aceclorism, acecrona, Aceffein, acemetacin, asprin (Acenterine), Acetal-SP (Aceclofenac—combination, ibuprofen, Acetyl-G, acetylsalicylate dl-lysine, acetylsalicylic acid, Acicot, Acifine, Acik, Aclocen, Acloflam-P, Aclomore, Aclon, A-CQ, ACS15, actarit, Actemra, Acthelea liofilizado, Actifast, Actimab-B, Actiquim, Actirin, Actis PLUS, activated leukocyte cell adhesion molecule antibody, Acular X, AD452, adalimumab, ADAMTS5 inhibitor, ADC1001, Adco-Diclofenac, Adco-Indomethacin, Adco-Meloxicam, Adco-Naproxen, Adco-Piroxicam, Adcort, Adco-Sulindac, adenosine triphosphate disodium, AdenosineA2a Receptor Agonist, Adimod, Adinos, Adioct, Adiodol, Adipoplus, adipose derived stem and/or regenerative cells, Adizen, Adpep, Advacan, Advagraf, Advel, Adwiflam, AEB071, Aental, Afenac, Affen Plus, Afiancen, Afinitor, Aflamin, Aflazacort, Aflogen, Afloxan, AFM15, AFM16, AFM17, AFM23, Afpred-Dexa, AFX200, AG011, Agafen, aganirsen, AGI1096, Agidex, AGS010, Agudol, A-Hydrocort, AIK1, AIN457, Airtal, AIT110, AJM300, ajulemic acid, AK106, AL-24-2A1, AL4-1A1, Ala Cort, Alanz, Albumin immune-globulin, alclometasone dipropionate, ALD518, aldesleukin, Aldoderma, alefacept, alemtuzumab, Alequel™, Alergolon, Alergosone, Aletraxon, Alfenac, Algason, Algin vek coat, Algioflex, Algirex, Algivin Plus, alicaforsen sodium, Alin, Alinia, Aliviodol, Aliviosin, alkaline phosphatase, ALKS6931, allantoin, Allbupen, Allmol, Allochrysine, allogeneic endothelial cells, allogeneic mesenchymal precursor cells, allogeneic mesenchymal stem cells, alminoprofen, alpha 1 antitrypsin, Alpha 7 nicotinic agonists, alpha amylase, alpha chymotrypsin, alpha fetoprotein, alpha linolenic acid, alpha-1-antitrypsin, α2β1 integrin inhibitors, Alphacort, Alphafen, alpha-hexidine, alpha-trypsin, Alphintern, Alpinamed mobility omega 3, Alpoxen, AL-Rev1, Alterase, ALX0061, ALX0761, ALXN1007, ALXN1102, AM3840, AM3876, AMAB, AMAP102, Amason, Ambene, AmbezimG, amcinonide, AME133v, Amecin, Ameloteks, A-Methapred, Amevive, AMG108, AMG139, AMG162, AMG181, AMG191, AMG220, AMG623, AMG674, AMG714, AMG719, AMG729, AMG827, Amidol, amifampridine phosphate, diclofenac (Emifenac®), Amimethacin, amiprilose hydrochloride, Amiprofen, Ammophos, Amoflam, AMP110, Ampikyy, Ampion, ampiroxicam, amtolmetin guacil, AMX256, AN6415, ANA004, ANA506, Anabu, Anacen, Anaflam, Anaflex ACI, Anaida, anakinra, Analgen Artritis, Anapan, Anaprox, Anavan, Anax, Anco, andrographis, Aneol, Anergix, Anervax.RA™ (therapeutic peptide vaccine), Anflene, ANG797, Anilixin, Anmerushin, Annexin 1 peptides, annexin A5, Anodyne, Ansaid, Anspirin, Antarene, anti BST2 antibody, anti C5a MAb, anti ILT7 antibody, anti VLA1 antibody, anti-alphal 1 antibody, anti-CD4 802-2, anti-CD86 monoclonal antibody, anti-chemokine, anti-DC-SIGN, anti-HMGB-1 MAb, anti-IL-18 Mab, anti-IL-1R MAb, anti-IL-1R MAb, anti-IL23 BRISTOL, anti-interleukin-1β antibody, anti-LIGHT antibody, anti-MIF antibody, anti-MIF antibody, anti-miR181a, antioxidant inflammation modulators, Antiphlamine, AntiRAGE MAb, antithrombin III, Anti-TIRC-7 MAb, Anusol-HC, Anyfen, AP105, AP1089, AP1189, AP401, AP501, apazone, APD334, Apentac, APG103, Apidone, apilimod mesylate, Apitac, Apitoxin, Apizel, APN inhibitor, apo-azathioprine, Apo-dexamethasone, ApoE mimetics, ApoFasL, apo-Indomethacin, apo-mefenamic, apo-methotrexate, apo-nabumetone, Apo-Napro-NA, apo-Naproxen, aponidin, apo-Phenylbutazone, apo-Piroxicam, apo-Sulin, Apo-Tenoxicam, apo-Tiaprofenic, Apranax, apremilast, apricoxib, Aprofen, Aprose, Aproxen, APX001 antibody, APX007 antibody, APY0201, AqvoDex, AQX108, AQX1125, AQX131135, AQX140, AQX150, AQX200, AQX356, AQXMN100, AQXMN106, ARA290, Arava, Arcalyst, Arcoxia, Arechin, Arflur, ARG098, ARG301, arginine aescin, arginine deiminase (pegylated), ARGX109 antibody, ARGX110, Arheuma, Aristocort, Aristospan, Ark-AP, ARN4026, Arofen, Aroff EZ, Arolef, Arotal, Arpibru, Arpimune, Arpu Shuangxin, ARQ101, Arrestin SP, Arrox, ARRY162, ARRY371797, ARRY614, ARRY872, ART621, Artamin, Arthfree, Artho Tech, Arthrexin, Arthrispray, Arthrotec, aeterna shark cartilage extract (Arthrovas™, Neoretna™, Psovascar™), Artifit, Artigo, Artin, Artinor, Artisid, Artoflex, Artren Hipergel, Artridol, Artrilase, Artrocaptin, Artrodiet, Artrofen, Artropan, Artrosil, Artrosilene, Artrotin, Artrox, Artyflam, Arzerra, AS604850, AS605858, Asacol, ASA-Grindeks, Asazipam, Aseclo, ASF1096, ASF1096, ASK8007, ASKP1240, ASLAN003, Asmo ID, Asonep, ASP015K, ASP2408, ASP2409, Aspagin, Aspeol, Aspicam, Aspirimex, AST120, astaxanthin, AstroCort, Aszes, AT002 antibody, AT007, AT008 antibody, AT008 antibody, AT010, AT1001, atacicept, Ataspin, Atepadene, Atgam, ATG-Fresenius, Athrofen, ATIO03, atiprimod, ATL1222, ATN103, ATN192, ATR107, Atri, Atrmin, Atrosab antibody, ATX3105, AU801, auranofin, Aurobin, Auropan, Aurothio, aurotioprol, autologous adipose derived regenerative cells, Autonec, Avandia, AVE9897, AVE9940, Avelox, Avent, AVI3378, Avloquin, AVP13546, AVP13748, AVP28225, AVX002, Axcel Diclofenac, Axcel Papain, Axen, AZ17, AZ175, Azacortid, AZA-DR, Azafrine, Azamun, Azanin, Azap, Azapin, Azapren, Azaprin, Azaram, Azasan, azathioprine, AZD0275, AZD0902, AZD2315, AZD5672, AZD6703, AZD7140, AZD8309, AZD8566, AZD9056, Azet, Azintrel, azithromycin, Az-od, Azofit, Azolid, Azoran, Azulene, Azulfidine, Azulfin, B1 antagonists, Baclonet, BAF312, BAFF Inhibitor, Bages, Baily S.P., Baleston, Balsolone, baminercept alfa, bardoxolone methyl, baricitinib, Barotase, Basecam, basiliximab, Baxmune, Baxo, BAY869766, BB2827, BCX34, BCX4208, Becfine, Beclate-C, Beclate-N, Beclolab Q, beclomethasone dipropionate, Beclorhin, Becmet-CG, Begita, Begti, belatacept, belimumab, Belosalic, Bemetson, Ben, Benevat, Benexam, Benflogin, Benisan, Benlysta, Benlysta, benorilate, Benoson, benoxaprofen, Bentol, benzydamine hydrochloride, Benzymin, Beofenac, Berafen, Berinert, Berlofen, Bertanel, Bestamine, Bestofen, Beta Nicip, Betacort, Betacorten G, Betafoam, beta-glucan, Betalar, Beta-M, Betamed, Betamesol, betamethasone, betamethasone dipropionate, betamethasone sodium, betamethasone sodium phosphate, betamethasone valerate, Betane, Betanex, Betapanthen, Betapar, Betapred, Betason, Betasonate, Betasone, Betatrinta, Betaval, Betazon, Betazone, Betesil, Betnecort, Betnesol, Betnovate, Bextra, BFPC13, BFPC18, BFPC21, BFPT6864, BG12, BG9924, BI695500, BI695501, BIA12, Big-Joint-D, BIIB023 antibody, Bi-ksikam, Bingo, BioBee, Bio-Cartilage, Bio-C-Sinkki, Biodexone, Biofenac, Bioreucam, Biosone, Biosporin, BIRB796, Bitnoval, Bitvio, Bivigam, BKT140, BKTP46, BL2030, BL3030, BL4020, BL6040, BL7060, BLI1300, blisibimod, Blokium B12, Blokium Gesic, Blokium, BMS066, BMS345541, BMS470539, BMS561392, BMS566419, BMS582949, BMS587101, BMS817399, BMS936557, BMS945429, BMS-A, BN006, BN007, BNP166, Bonacort, Bonas, bone marrow stromal cell antigen 2 antibody, Bonflex, Bonifen, Boomiq, Borbit, Bosong, BRO2001, BR3-FC, Bradykinin B1 Receptor Antagonist, Bredinin, Brexecam, Brexin, Brexodin, briakinumab, Brimani, briobacept, Bristaflam, Britten, Broben, brodalumab, Broen-C, bromelains, Bromelin, Bronax, Bropain, Brosiral, Bruace, Brufadol, Brufen, Brugel, Brukil, Brusil, BT061, BTI9, BTK kinase inhibitors, BTT1023 antibody, BTT1507, bucillamine, Bucillate, Buco Reigis, bucolome, Budenofalk, budesonide, Budex, Bufect, Bufencon, Bukwang Ketoprofen, Bunide, Bunofen, Busilvex, busulfan, Busulfex, Busulipo, Butartrol, Butarut B12, Butasona, Butazolidin, Butesone, Butidiona, BVX10, BXL628, BYM338, B-Zone, Cl esterase inhibitor, C243, c4462, c5997, C5aQb, c7198, c9101, C9709, c9787, CAB101, cadherin 11 antibody, caerulomycin A, CAL263, Calcort, Calmatel, CAM3001, Camelid Antibodies, Camlox, Camola, Campath, Camrox, Camtenam, canakinumab, candida albicans antigen, Candin, cannabidiol, CAP1.1, CAP1.2, CAP2.1, CAP2.2, CAP3.1, CAP3.2, Careram, Carimune, Cariodent, Cartifix, CartiJoint, Cartilago, Cartisafe-DN, Cartishine, Cartivit, Cartril-S, Carudol, CaspaCIDe, CaspaCIDe, Casyn, CAT1004, CAT1902, CAT2200, Cataflam, Cathepsin S inhibitor, Catlep, CB0114, CB2 agonist, CC0478765, CC10004, CC10015, CC1088, CC11050, CC13097, CC15965, CC16057, CC220, CC292, CC401, CC5048, CC509, CC7085, CC930, CCR1 antagonist, CCR6 inhibitor, CCR7 antagonist, CCRL2 antagonist, CCX025, CCX354, CCX634, CD Diclofenac, CD102, CD103 antibody, CD103 antibody, CD137 antibody, CD16 antibody, CD18 antibody, CD19 antibody, CD1d antibody, CD20 antibody, CD200Fc, CD209 antibody, CD24, CD3 antibody, CD30 antibody, CD32A antibody, CD32B antibody, CD4 antibody, CD40 ligand, CD44 antibody, CD64 antibody, CDC839, CDC998, CDIM4, CDIM9, CDK9-Inhibitor, CDP146, CDP323, CDP484, CDP6038, CDP870, CDX1135, CDX301, CE224535, Ceanel, Cebedex, Cebutid, Ceclonac, Ceex, CEL2000, Celact, Celbexx, Celcox, Celebiox, Celebrex, Celebrin, Celecox, celecoxib, Celedol, Celestone, Celevex, Celex, CELG4, Cell adhesion molecule antagonists, CellCept, Cellmune, Celosti, Celoxib, Celprot, Celudex, cenicriviroc mesylate, cenplacel-1, CEP11004, CEP37247, CEP37248, Cephyr, Ceprofen, Certican, certolizumab pegol, Cetofenid, Cetoprofeno, cetylpyridinium chloride, CF101, CF402, CF502, CG57008, CGEN15001, CGEN15021, CGEN15051, CGEN15091, CGEN25017, CGEN25068, CGEN40, CGEN54, CGEN768, CGEN855, CGI1746, CGI560, CGI676, Cgtx-Peptides, CH1504, CH4051, CH4446, chaperonin 10, chemokine C-C motif ligand 2, chemokine C-C motif ligand 2 antibody, chemokine C-C motif ligand 5 antibody, chemokine C-C motif receptor 2 antibody, chemokine C-C motif receptor 4 antibody, chemokine C-X-C motif ligand 10 antibody, chemokine C-X-C motif ligand 12 aptamer, Chemotaxis Inhibitor, Chillmetacin, chitinase 3-like 1, Chlocodemin, Chloquin, chlorhexidine gluconate, chloroquine phosphate, choline magnesium trisalicylate, chondroitin sulfate, Chondroscart, CHR3620, CHR4432, CHR5154, Chrysalin, Chuanxinlian, Chymapra, Chymotase, chymotrypsin, Chytmutrip, CI202, CI302, Cicloderm-C, Ciclopren, Cicporal, Cilamin, Cimzia, cinchophen, cinmetacin, cinnoxicam, Cinoderm, Cinolone-S, Cinryze, Cipcorlin, cipemastat, Cipol-N, Cipridanol, Cipzen, Citax F, Citogan, Citoken T, Civamide, CJ042794, CJ14877, c-Kit monoclonal antibody, cladribine, Clafen, Clanza, Claversal, clazakizumab, Clearoid, Clease, Clevegen, Clevian, Clidol, Clindac, Clinoril, Cliptol, Clobenate, Clobequad, clobetasol butyrate, clobetasol propionate, Clodol, clofarabine, Clofen, Clofenal LP, Clolar, Clonac, Clongamma, clonixin lysine, Clotasoce, Clovacort, Clovana, Cloxin, CLT001, CLT008, C-MAF Inhibitor, CMPX1023, Cnac, CND0201, CNI1493, CNTO136, CNTO148, CNT01959, Cobefen, CoBenCoDerm, Cobix, Cofenac, Cofenac, COG241, COL179, colchicine, Colchicum Dispert, Colchimax, Colcibra, Coledes A, Colesol, Colifoam, Colirest, collagen, type V, Comcort, complement component (3b/4b) receptor 1, Complement Component Cls Inhibitors, complement component C3, complement factor 5a receptor antibody, complement factor 5a receptor antibody, complement factor D antibody, Condrosulf, Condrotec, Condrothin, conestat alfa, connective tissue growth factor antibody, Coolpan, Copaxone, Copiron, Cordefla, Corhydron, Cort S, Cortan, Cortate, Cort-Dome, Cortecetine, Cortef, Corteroid, Corticap, Corticas, Cortic-DS, corticotropin, Cortiderm, Cortidex, Cortiflam, Cortinet M, Cortinil, Cortipyren B, Cortiran, Cortis, Cortisolu, cortisone acetate, Cortival, Cortone acetate, Cortopin, Cortoral, Cortril, Cortypiren, Cosamine, Cosone, cosyntropin, COT Kinase Inhibitor, Cotilam, Cotrisone, Cotson, Covox, Cox B, COX-²/₅-LO Inhibitors, Coxeton, Coxflam, Coxicam, Coxitor, Coxtral, Coxypar, CP195543, CP412245, CP424174, CP461, CP629933, CP690550, CP751871, CPSI2364, C-quin, CR039, CR074, CR106, CRA102, CRAC channel inhibitor, CRACM ion channel inhibitor, Cratisone, CRB15, CRC4273, CRC4342, C-reactive protein 2-methoxyethyl phosphorothioate oligonucleotide, CreaVax-RA, CRH modulators, critic-aid, Crocam, Crohnsvax, Cromoglycic acid, cromolyn sodium, Cronocorteroid, Cronodicasone, CRTX803, CRx119, CRx139, CRx150, CS502, CS670, CS706, CSF1R Kinase Inhibitors, CSL324, CSL718, CSL742, CT112, CT1501R, CT200, CT2008, CT2009, CT3, CT335, CT340, CT5357, CT637, CTP05, CTP10, CT-P13, CTP17, Cuprenil, Cuprimine, Cuprindo, Cupripen, Curaquin, Cutfen, CWF0808, CWP271, CX1020, CX1030, CX1040, CX5011, Cx611, Cx621, Cx911, CXC chemokine receptor 4 antibody, CXCL13 antibodies, CXCR3 antagonists, CXCR4 antagonist, Cyathus 1104 B, Cyclo-2, Cyclocort, cyclooxygenase-2 inhibitor, cyclophosphamide, Cyclorine, Cyclosporin A Prodrug, Cyclosporin analogue A, cyclosporine, Cyrevia, Cyrin CLARIS, CYT007TNFQb, CYT013IL1bQb, CYT015IL17Qb, CYT020TNFQb, CYT107, CYT387, CYT99007, cytokine inhibitors, Cytopan, Cytoreg, CZC24832, D1927, D9421C, daclizumab, danazol, Danilase, Dantes, Danzen, dapsone, Dase-D, Daypro, Daypro Alta, Dayrun, Dazen, DB295, DBTP2, D-Cort, DD1, DD3, DE096, DE098, Debio0406, Debio0512, Debio0615, Debio0618, Debio1036, Decaderm, Decadrale, Decadron, Decadronal, Decalon, Decan, Decason, Decdan, Decilone, Declophen, Decopen, Decorex, Decorten, Dedema, Dedron, Deexa, Defcort, De-flam, Deflamat, Deflan, Deflanil, Deflaren, Deflaz, deflazacort, Defnac, Defnalone, Defnil, Defosalic, Defsure, Defza, Dehydrocortison, Dekort, Delagil, delcasertib, delmitide, Delphicort, Deltacorsolone prednisolone (Deltacortril), Deltafluorene, Deltasolone, Deltasone, Deltastab, Deltonin, Demarin, Demisone, Denebola, denileukin diftitox, denosumab, Denzo, Depocortin, Depo-medrol, Depomethotrexate, Depopred, Deposet, Depyrin, Derinase, Dermol, Dermolar, Dermonate, Dermosone, Dersone, Desketo, desonide, desoxycorticosterone acetate, Deswon, Dexa, Dexabene, Dexacip, Dexacort, dexacortisone, Dexacotisil, dexadic, dexadrin, Dexadron, Dexafar, Dexahil, Dexalab, Dexalaf, Dexalet, Dexalgen, dexallion, dexalocal, Dexalone, Dexa-M, Dexamecortin, Dexamed, Dexamedis, dexameral, Dexameta, dexamethasone, dexamethasone acetate, dexamethasone palmitate, dexamethasone phosphate, dexamethasone sodium metasulfobenzoate, dexamethasone sodium phosphate, Dexamine, Dexapanthen, Dexa-S, Dexason, Dexatab, Dexatopic, Dexaval, Dexaven, Dexazolidin, Dexazona, Dexazone, Dexcor, Dexibu, dexibuprofen, Dexico, Dexifen, Deximune, dexketoprofen, dexketoprofen trometamol, Dexmark, Dexomet, Dexon I, Dexonalin, Dexonex, Dexony, Dexoptifen, Dexpin, Dextan-Plus, dextran sulfate, Dezacor, Dfz, diacerein, Diannexin, Diastone, Dicarol, Dicasone, Dicknol, Diclo, Diclobon, Diclobonse, Diclobonzox, Diclofast, Diclofen, diclofenac, diclofenac beta-dimethylaminoethanol, diclofenac deanol, diclofenac diethylamine, diclofenac epolamine, diclofenac potassium, diclofenac resinate, diclofenac sodium, Diclogen AGIO, Diclogen Plus, Diclokim, Diclomed, Diclo-NA, Diclonac, Dicloramin, Dicloran, Dicloreum, Diclorism, Diclotec, Diclovit, Diclowal, Diclozem, Dico P, Dicofen, Dicoliv, Dicorsone, Dicron, Dicser, Difena, Diffutab, diflunisal, dilmapimod, Dilora, dimethyl sulfone, Dinac, D-Indomethacin, Dioxaflex Protect, Dipagesic, Dipenopen, Dipexin, Dipro AS, Diprobeta, Diprobetasone, Diproklenat, Dipromet, Dipronova, Diprosone, Diprovate, Diproxen, Disarmin, Diser, Disopain, Dispain, Dispercam, Distamine, Dizox, DLT303, DLT404, DM199, DM99, DMI9523, dnaJP1, DNX02070, DNX04042, DNX2000, DNX4000, docosanol, Docz-6, Dolamide, Dolaren, Dolchis, Dolex, Dolflam, Dolfre, Dolgit, Dolmax, Dolmina, Dolo Ketazon, Dolobest, Dolobid, Doloc, Dolocam, Dolocartigen, Dolofit, Dolokind, Dolomed, Dolonac, Dolonex, Dolotren, Dolozen, Dolquine, Dom0100, Dom0400, Dom0800, Domet, Dometon, Dominadol, Dongipap, Donica, Dontisanin, doramapimod, Dorixina Relax, Dormelox, Dorzine Plus, Doxatar, Doxtran, DP NEC, DP4577, DP50, DP6221, D-Penamine, DPIV/APN Inhibitors, DR1 Inhibitors, DR4 Inhibitors, DRA161, DRA162, Drenex, DRF4848, DRL15725, Drossadin, DSP, Duexis, Duo-Decadron, Duoflex, Duonase, DV1079, DV1179, DWJ425, DWP422, Dymol, DYN15, Dynapar, Dysmen, E5090, E6070, Easy Dayz, Ebetrexat, EBI007, ECO286, EC0565, EC0746, Ecax, echinacea purpurea extract, EC-Naprosyn, Econac, Ecosprin 300, Ecosprin 300, Ecridoxan, eculizumab, Edecam, efalizumab, Efcortesol, Effigel, Eflagen, Efridol, EGFR Antibody, EGS21, eIF5A1 siRNA, Ekarzin, elafin, Eldoflam, Elidel, Eliflam, Elisone, Elmes, Elmetacin, ELND001, ELND004, elocalcitol, Elocom, elsibucol, Emanzen, Emcort, Emifen, Emifenac, emorfazone, Empynase, emricasan, Emtor, Enable, Enbrel, Enceid, EncorStat, Encortolon, Encorton, Endase, Endogesic, Endoxan, Enkorten, Ensera, Entocort, Enzylan, Epanova, Eparang, Epatec, Epicotil, epidermal growth factor receptor 2 antibody, epidermal growth factor receptor antibody, Epidixone, Epidron, Epiklin, EPPA1, epratuzumab, EquiO, Erac, Erazon, ERB041, ERB196, Erdon, EryDex, escherichia coli enterotoxin B subunit, Escin, E-Selectin Antagonists, Esfenac, ESN603, esonarimod, Esprofen, estetrol, Estopein, Estrogen Receptor beta agonist, etanercept, etaracizumab, ETC001, ethanol propolis extract, ETI511, etiprednol dicloacetate, Etodin, Etodine, Etodol, etodolac, Etody, etofenamate, Etol Fort, Etolac, Etopin, etoricoxib, Etorix, Etosafe, Etova, Etozox, Etura, Eucob, Eufans, eukaryotic translation initiation factor 5A oligonucleotide, Eunac, Eurocox, Eurogesic, everolimus, Evinopon, EVT401, Exaflam, EXEL9953, Exicort, Expen, Extra Feverlet, Extrapan, Extrauma, Exudase, F16, F991, Falcam, Falcol, Falzy, Farbovil, Farcomethacin, Farnerate, Farnezone, Farnezone, Farotrin, fas antibody, Fastflam, FasTRACK, Fastum, Fauldmetro, FcgammaRIA antibody, FE301, Febrofen, Febrofid, felbinac, Feldene, Feldex, Feloran, Felxicam, Fenac, Fenacop, Fenadol, Fenaflan, Fenamic, Fenaren, Fenaton, Fenbid, fenbufen, Fengshi Gutong, Fenicort, Fenopine, fenoprofen calcium, Fenopron, Fenris, Fensupp, Fenxicam, fepradinol, Ferovisc, Feverlet, fezakinumab, FG3019, FHT401, FHTCT4, FID114657, figitumumab, Filexi, filgrastim, Fillase, Final, Findoxin, fingolimod hydrochloride, firategrast, Firdapse, Fisiodar, Fivasa, FK778, Flacoxto, Fladalgin, Flagon, Flamar, Flamcid, Flamfort, Flamide, Flaminase, Flamirex Gesic, Flanid, Flanzen, Flaren, Flaren, Flash Act, Flavonoid Anti-inflammatory Molecule, Flebogamma DIF, Flenac, Flex, Flexafen 400, Flexi, Flexidol, Flexium, Flexon, Flexono, Flogene, Flogiatrin B12, Flogomin, Flogoral, Flogosan, Flogoter, Flo-Pred, Flosteron, Flotrip Forte, Flt3 inhibitors, fluasterone, Flucam, Flucinar, fludrocortisone acetate, flufenamate aluminum, flumethasone, Flumidon, flunixin, fluocinolone, fluocinolone acetonide, fluocinonide, fluocortolone, Fluonid, fluorometholone, Flur, flurbiprofen, Fluribec, Flurometholone, Flutal, fluticasone, fluticasone propionate, Flutizone, Fluzone, FM101 antibody, fms-related tyrosine kinase 1 antibody, Folitrax, fontolizumab, formic acid, Fortecortin, Fospeg, fostamatinib disodium, FP1069, FP13XX, FPA008, FPA031, FPT025, FR104, FR167653, Framebin, Frime, Froben, Frolix, FROUNT Inhibitors, Fubifen PAP, Fucole ibuprofen, Fulamotol, Fulpen, Fungifin, Furotalgin, fusidate sodium, FX002, FX141L, FX201, FX300, FX87L, Galectin modulators, gallium maltolate, Gamimune N, Gammagard, Gamma-I.V., GammaQuin, Gamma-Venin, Gamunex, Garzen, Gaspirin, Gattex, GBR500, GBR500 antibody, GBT009, G-CSF, GED0301, GED0414, Gefenec, Gelofen, Genepril, Gengraf, Genimune, Geniquin, Genotropin, Genz29155, Gerbin, Gerbin, gevokizumab, GF01564600, Gilenia, Gilenya, givinostat, GL0050, GL2045, glatiramer acetate, Globulin, Glortho Forte, Glovalox, Glovenin-I, GLPG0259, GLPG0555, GLPG0634, GLPG0778, GLPG0974, Gluco, Glucocerin, glucosamine, glucosamine hydrochloride, glucosamine sulfate, Glucotin, Gludex, Glutilage, GLY079, GLY145, Glycanic, Glycefort up, Glygesic, Glysopep, GMCSF Antibody, GMI1010, GMI1011, GMI1043, GMR321, GN4001, Goanna Salve, Goflex, gold sodium thiomalate, golimumab, GP2013, GPCR modulator, GPR15 Antagonist, GPR183 antagonist, GPR32 antagonist, GPR83 antagonist, G-protein Coupled Receptor Antagonists, Graceptor, Graftac, granulocyte colony-stimulating factor antibody, granulocyte-macrophage colony-stimulating factor antibody, Gravx, GRC4039, Grelyse, GS101, GS9973, GSC100, GSK1605786, GSK1827771, GSK2136525, GSK2941266, GSK315234, GSK681323, GT146, GT442, Gucixiaotong, Gufisera, Gupisone, gusperimus hydrochloride, GW274150, GW3333, GW406381, GW856553, GWB78, GXP04, Gynestrel, Haloart, halopredone acetate, Haloxin, HANALL, Hanall Soludacortin, Havisco, Hawon Bucillamin, HB802, HC31496, HCQ 200, HD104, HD203, HD205, HDAC inhibitor, HE2500, HE3177, HE3413, Hecoria, Hectomitacin, Hefasolon, Helen, Helenil, HemaMax, Hematom, hematopoietic stem cells, Hematrol, Hemner, Hemril, heparinoid, Heptax, HER2 Antibody, Herponil, hESC Derived Dendritic Cells, hESC Derived Hematopoietic stem cells, Hespercorbin, Hexacorton, Hexadrol, hexetidine, Hexoderm, Hexoderm Salic, HF0220, HF1020, HFT-401, hG-CSFR ED Fc, Hiberna, high mobility group box 1 antibody, Hiloneed, Hinocam, hirudin, Hirudoid, Hison, Histamine H4 Receptor Antagonist, Hitenercept, Hizentra, HL036, HL161, HMPL001, HMPL004, HMPL004, HMPL011, HMPL342, HMPL692, honey bee venom, Hongqiang, Hotemin, HPH116, HTI101, HuCAL Antibody, Human adipose mesenchymal stem cells, anti-MHC class II monoclonal antibody, Human Immunoglobulin, Human Placenta Tissue Hydrolysate, HuMaxCD4, HuMax-TAC, Humetone, Humicade, Humira, Huons Betamethasone sodium phosphate, Huons dexamethasone sodium phosphate, Huons Piroxicam, Huons Talniflumate, Hurofen, Huruma, Huvap, HuZAF, HX02, Hyalogel, hyaluronate sodium, hyaluronic acid, hyaluronidase, Hyaron, Hycocin, Hycort, Hy-Cortisone, hydrocortisone, hydrocortisone acetate, hydrocortisone butyrate, hydrocortisone hemisuccinate, hydrocortisone sodium phosphate, hydrocortisone sodium succinate, Hydrocortistab, Hydrocortone, Hydrolin, Hydroquine, Hydro-Rx, Hydrosone HIKMA, hydroxychloroquine, hydroxychloroquine sulfate, Hylase Dessau, HyMEX, Hypen, HyQ, Hysonate, HZN602, I.M.75, IAP Inhibitors, Ibalgin, Ibalgin, Ibex, ibrutinib, IBsolvMIR, Ibu, Ibucon, Ibudolor, Ibufen, Ibuflam, Ibuflex, Ibugesic, Ibu-Hepa, Ibukim, Ibumal, Ibunal, Ibupental, Ibupril, Ibuprof, ibuprofen, Ibuscent, Ibusoft, Ibusuki Penjeong, Ibususpen, Ibutard, Ibutop, Ibutop, Ibutrex, IC487892, ichthammol, ICRAC Blocker, IDEC131, IDECCE9.1, Ides, Idicin, Idizone, IDN6556, Idomethine, IDR1, Idyl SR, Ifen, iguratimod, IK6002, IKK-beta inhibitor, IL17 Antagonist, IL-17 Inhibitor, IL-17RC, IL18, IL1Hyl, IL1R1, IL-23 Adnectin, IL23 Inhibitor, IL23 Receptor Antagonist, IL-31 mAb, IL-6 Inhibitor, IL6Qb, Ilacox, Ilaris, ilodecakin, ILV094, ILV095, Imaxetil, IMD0560, IMD2560, Imesel Plus, Iminoral, Immodin, IMMU103, IMMU106, Immucept, Immufine, Immunex Syrup, immunoglobulin, immunoglobulin G, Immunoprin, ImmunoRel, Immurin, IMO8400, IMP731 antibody, Implanta, Imunocell, Imuran, Imurek, Imusafe, Imusporin, Imutrex, IN0701, Inal, INCB039110, INCB18424, INCB28050, INCB3284, INCB3344, Indexon, Indic, Indo, Indo-A, Indobid, Indo-Bros, Indocaf, Indocarsil, Indocid, Indocin, Indomehotpas, Indomen, Indomet, Indometacin, indomethacin, Indomethasone, Indometin, Indomin, Indopal, Indoron, Indotroxin, INDUS830, INDUS83030, Infladase, Inflamac, Inflammasome inhibitor, Inflavis, Inflaxen, Inflectra, infliximab, Ingalipt, Inicox dp, Inmecin, Inmunoartro, Innamit, InnoD06006, IN07997, Inocin, Inoten, Inovan, Inpra, Inside Pap, Insider-P, Instacyl, Instracool, Intafenac, Intaflam, Inteban, Inteban Spansule, integrin, alpha 1 antibody, integrin, alpha 2 antibody, Intenurse, interferon alfa, interferon beta-1a, interferon gamma, interferon gamma antibody, Interking, interleukin 1 Hyl, interleukin 1 antibody, interleukin 1 receptor antibody, interleukin 1, beta antibody, interleukin 10, interleukin 10 antibody, interleukin 12, interleukin 12 antibody, interleukin 13 antibody, interleukin 15 antibody, interleukin 17 antibody, interleukin 17 receptor C, interleukin 18, interleukin 18 binding protein, interleukin 18 antibody, interleukin 2 receptor, alpha antibody, interleukin 20 antibody, Interleukin 21 mAb, interleukin 23 aptamer, interleukin 31 antibody, interleukin 34, Interleukin 6 Inhibitor, interleukin 6 antibody, interleukin 6 receptor antibody, interleukin 7, interleukin 7 receptor antibody, interleukin 8, interleukin 8 antibody, interleukin-18 antibody, Intidrol, Intradex, Intragam P, Intragesic, Intraglobin F, Intratect, Inzel, Iomab B, IOR-T3, IP751, IPH2201, IPH2301, IPH24, IPH33, IPI145, Ipocort, IPP201007, I-Profen, Iprox, Ipson, Iputon, IRAK4 Inhibitor, Iremod, Irtonpyson, IRX3, IRX5183, ISA247, ISIS104838, ISIS2302, ISISCRPRx, Ismafron, IsoQC inhibitor, Isox, ITF2357, Iveegam EN, Ivepred, WIG-SN, IW001, Izilox, J607Y, J775Y, JAK Inhibitor, JAK3 inhibitor, JAK3 kinase inhibitor, JI3292, JI4135, Jinan Lida, JNJ10329670, JNJ18003414, JNJ26528398, JNJ27390467, JNJ28838017, JNJ31001958, JNJ38518168, JNJ39758979, JNJ40346527, JNJ7777120, JNT-Plus, Joflam, Joint Glucosamin, Jointec, Jointstem, Joinup, JPE1375, JSM10292, JSM7717, JSM8757, JTE051, JTE052, JTE522, JTE607, Jusgo, K412, K832, Kaflam, KAHR101, KAHR102, KAI9803, Kalymin, Kam Predsol, Kameton, KANAb071, Kappaproct, KAR2581, KAR3000, KAR3166, KAR4000, KAR4139, KAR4141, KB002, KB003, KD7332, KE298, keliximab, Kemanat, Kemrox, Kenacort, Kenalog, Kenaxir, Kenketsu Venoglobulin-IH, Keplat, Ketalgipan, Keto Pine, Keto, Ketobos, Ketofan, Ketofen, Ketolgan, Ketonal, Ketoplus Kata Plasma, ketoprofen, Ketores, Ketorin, ketorolac, ketorolac tromethamine, Ketoselect, Ketotop, Ketovail, Ketricin, Ketroc, Ketum, Keyi, Keyven, KF24345, K-Fenac, K-Fenak, K-Gesic, Kifadene, Kilcort, Kildrol, KIM127, Kimotab, Kinase Inhibitor 4SC, Kinase N, Kincort, Kindorase, Kineret, Kineto, Kitadol, Kitex, Kitolac, KLK1 Inhibitor, Klofen-L, Klotaren, KLS-40or, KLS-40ra, KM277, Knavon, Kodolo orabase, Kohakusanin, Koide, Koidexa, Kolbet, Konac, Kondro, Kondromin, Konshien, Kontab, Kordexa, Kosa, Kotase, KPE06001, KRP107, KRP203, KRX211, KRX252, KSB302, K-Sep, Kv 1.3 Blocker, Kv1.3 4SC, Kv1.3 inhibitor, KVK702, Kynol, L156602, Labizone, Labohydro, Labopen, Lacoxa, Lamin, Lamit, Lanfetil, laquinimod, larazotide acetate, LAS186323, LAS187247, LAS41002, Laticort, LBEC0101, LCP3301, LCP-Siro, LCP-Tacro, LCsA, LDP392, Leap-S, Ledercort, Lederfen, Lederlon, Lederspan, Lefenine, leflunomide, Leflux, Lefno, Lefra, Leftose, Lefumide, Lefunodin, Lefva, lenalidomide, lenercept, LentiRA, LE015520, Leodase, Leukine, Leukocyte function-associated antigen-1 antagonist, leukocyte immunoglobulin-like receptor, subfamily A, member 4 antibody, Leukothera, leuprolide acetate, levalbuterol, levomenthol, LFA-1 Antagonist, LFA451, LFA703, LFA878, LG106, LG267 Inhibitors, LG688 Inhibitors, LGD5552, Li Life, LidaMantle, Lidex, lidocaine, lidocaine hydrochloride, Lignocaine hydrochloride, LIM0723, LIM5310, Limethason, Limus, Limustin, Lindac, Linfonex, Linola acute, Lipcy, lisofylline, Listran, Liver X Receptor modulator, Lizak, LP1207, LJP920, Lobafen, Lobu, Locafluo, Localyn, Locaseptil-Neo, Locpren, Lodine, Lodotra, Lofedic, Loflam, Lofnac, Lolcam, Lonac, lonazolac calcium, Loprofen, Loracort, Lorcam, Lorfenamin, Lorinden Lotio, Lorncrat, lornoxicam, Lorox, losmapimod, loteprednol etabonate, Loteprednol, Lotirac, Low Molecular Ganoderma Lucidum Polysaccharide, Loxafen, Loxfenine, Loxicam, Loxofen, Loxonal, Loxonin, loxoprofen sodium, Loxoron, LP183A1, LP183A2, LP204A1, LPCN1019, LT1942, LT1964, LTNS101, LTNS103, LTNS106, LTNS108, LTS1115, LTZMP001, Lubor, lumiracoxib, Lumitect, LX2311, LX2931, LX2932, LY2127399, LY2189102, LY2439821, LY294002, LY3009104, LY309887, LY333013, lymphocyte activation gene 3 antibody, Lymphoglobuline, Lyser, lysine aspirin, Lysobact, Lysoflam, Lysozyme hydrochloride, M3000, M834, M923, mAb hG-CSF, MABP1, macrophage migration inhibitory factor antibody, Maitongna, Majamil prolongatum, major histocompatibility complex class II DR antibody, major histocompatibility complex class II antibody, Malidens, Malival, mannan-binding lectin, mannan-binding lectin-associated serine protease-2 antibody, MapKap Kinase 2 Inhibitor, maraviroc, Marlex, masitinib, Maso, MASP2 antibody, MAT304, Matrix Metalloprotease Inhibitor, mavrilimumab, Maxiflam, Maxilase, Maximus, Maxisona, Maxius, Maxpro, Maxrel, Maxsulid, Maxyl2, Maxy30, MAXY4, Maxy735, Maxy740, Mayfenamic, MB11040, MBPY003b, MCAF5352A, McCam, McRofy, MCS18, MD707, MDAM, MDcort, MDR06155, MDT012, Mebicam, Mebuton, meclofenamate sodium, Meclophen, Mecox, Medacomb, Medafen, Medamol, Medesone, MEDI2070, MEDI5117, MEDI541, MEDI552, MEDI571, Medicox, Medifen, Medisolu, Medixon, Mednisol, Medrol, Medrolon, medroxyprogesterone acetate, Mefalgin, mefenamic acid, Mefenix, Mefentan, Meflen, Mefnetra forte, Meftagesic-DT, Meftal, Megakaryocyte Growth and Development Factor, Megaspas, Megaster, megestrol acetate, Meite, Meksun, Melbrex, Melcam, Melcam, Melflam, Melic, Melica, Melix, Melocam, Melocox, Mel-One, Meloprol, Melosteral, Melox, Meloxan, Meloxcam, Meloxic, Meloxicam, Meloxifen, Meloxin, Meloxiv, Melpred, Melpros, Melurjin, Menamin, Menisone, Menthomketo, Menthoneurin, Mentocin, Mepa, Mepharen, meprednisone, Mepresso, Mepsolone, mercaptopurine, Mervan, Mesadoron, mesalamine, Mesasal, Mesatec, Mesenchymal Precursor Cells, mesenchymal stem cell, Mesipol, Mesren, Mesulan, Mesulid, Metacin, Metadaxan, Metaflex, Metalcaptase, metalloenzyme inhibitors, Metapred, Metax, Metaz, Meted, Metedic, Methacin, Methaderm, Methasone, Methotrax, methotrexate, methotrexate sodium, Methpred, Methyl prednisolone acetate, methyl salicylate, methyl sulphonyl methane, Methylon, Methylpred, methylprednisolone, methylprednisolone acetate, methylprednisolone sodium succinate, methylprednisolone succinate, Methylprednisolone, Methysol, Metindol, Metoart, Metoject, Metolate, Metoral, Metosyn, Metotab, Metracin, Metrex, metronidazole, Metypred, Mevamox, Mevedal, Mevilox, Mevin SR, Mexilal, Mexpharm, Mext, Mextran, MF280, M-FasL, MHC class II beta chain peptide, Micar, Miclofen, Miclofenac, Micofenolato Mofetil, Micosone, Microdase, microRNA 181a-2 oligonucleotide, MIF Inhibitors, MIFQb, MIKA-Ketoprofen, Mikametan, milodistim, Miltax, Minafen, Minalfen, Minalfene, Minesulin, Minocort, Mioflex, Miolox, Miprofen, Miridacin, Mirloks, Misoclo, Misofenac, MISTB03, MISTB04, Mitilor, mizoribine, MK0359, MK0812, MK0873, MK2 Inhibitors, MK50, MK8457, MK8808, MKC204, MLN0002, MLN0415, MLN1202, MLN273, MLN3126, MLN3701, MLN3897, MLNM002, MM093, MM7XX, MN8001, Mobic, Mobicam, Mobicox, Mobifen Plus, Mobilat, Mobitil, Mocox, Modigraf, Modrasone, Modulin, Mofecept, Mofetyl, mofezolac sodium, Mofilet, Molace, molgramostim, Molslide, Momekin, Momen Gele, Moment 100, Momesone, Momesun, Mometamed, mometasone, mometasone furoate, Monimate, monosodium alpha-luminol, Mopik, MOR103, MOR104, MOR105, MOR208 antibody, MORAb022, Moricam, morniflumate, Mosuolit, Motoral, Movaxin, Mover, Movex, Movix, Movoxicam, Mox Forte, Moxen, moxifloxacin hydrochloride, Mozobil, MP, MP0210, MP0270, MP1000, MP1031, MP196, MP435, MPA, mPGES-1 inhibitor, MPSS, MRX7EAT, MSL, MT203, MT204, mTOR Inhibitor, MTRX1011A, Mucolase, Multicort, MultiStem, muramidase, muramidase, muramidase hydrochloride, muromonab-CD3, Muslax, Muspinil, Mutaze, Muvera, MX68, Mycept, Mycocell, Mycocept, Mycofenolatmofetil Actavis, Mycofet, Mycofit, Mycolate, Mycoldosa, Mycomun, Myconol, mycophenolate mofetil, mycophenolate sodium, mycophenolic acid, Mycotil, myeloid progenitor cells, Myfenax, Myfetil, Myfortic, Mygraft, Myochrysine, Myocrisin, Myprodol, Mysone, nab-Cyclosporine, Nabentac, nabiximols, Nabton, Nabuco, Nabucox, Nabuflam, Nabumet, nabumetone, Nabuton, Nac Plus, Nacta, Nacton, Nadium, Naklofen SR, NAL1207, NAL1216, NAL1219, NAL1268, NAL8202, Nalfon, Nalgesin S, namilumab, Namsafe, nandrolone, Nanocort, Nanogam, Nanosomal Tacrolimus, Napageln, Napilac, Naprelan, Napro, Naprodil, Napronax, Napropal, Naproson, Naprosyn, Naproval, Naprox, naproxen, naproxen sodium, Naproxin, Naprozen, Narbon, Narexsin, Naril, Nasida, natalizumab, Naxdom, Naxen, Naxin, Nazovel, NC2300, ND07, NDC01352, Nebumetone, NecLipGCSF, Necsulide, Necsunim, Nelsid-S, Neo Clobenate, Neo Swiflox FC, Neocoflan, Neo-Drol, Neo-Eblimon, Neo-Hydro, Neoplanta, Neoporine, Neopreol, Neoprox, Neoral, Neotrexate, Neozen, Nepra, Nestacort, Neumega, Neupogen, Neuprex, Neurofenac, Neurogesic, Neurolab, Neuroteradol, Neuroxicam, Neutalin, neutrazumab, Neuzym, New Panazox, Newfenstop, NewGam, Newmafen, Newmatal, Newsicam, NEX1285, sFcRIIB, Nextomab, NF-kappaB Inhibitor, NF-kB inhibitor, NGD20001, NHP554B, NHP554P, NI0101 antibody, NI0401, NI0501 antibody, NI0701, NI071, NI1201 antibody, NI1401, Nicip, Niconas, Nicool, NiCord, Nicox, Niflumate, Nigaz, Nikam, Nilitis, Nimace, Nimaid, Nimark-P, Nimaz, Nimcet Juicy, Nime, Nimed, Nimepast, nimesulide, Nimesulix, Nimesulon, Nimica Plus, Nimkul, Nimlin, Nimnat, Nimodol, Nimpidase, Nimsaid-S, Nimser, Nimsy-SP, Nimupep, Nimusol, Nimutal, Nimuwin, Nimvon-S, Nincort, Niofen, Nipan, Nipent, Nise, Nisolone, Nisopred, Nisoprex, Nisulid, nitazoxanide, Nitcon, nitric oxide, Nizhvisal B, Nizon, NL, NMR1947, NN8209, NN8210, NN8226, NN8555, NN8765, NN8828, NNC014100000100, NNC051869, Noak, Nodevex, Nodia, Nofenac, Noflagma, Noflam, Noflamen, Noflux, Non-antibacterial Tetracyclines, Nonpiron, Nopain, Normferon, Notpel, Notritis, Novacort, Novagent, Novarin, Novigesic, NOXA12, NOXD19, Noxen, Noxon, NPI1302a-3, NPI1342, NPI1387, NPI1390, NPRCS1, NPRCS2, NPRCS3, NPRCS4, NPRCSS, NPRCS6, NPS3, NPS4, nPT-ery, NU3450, nuclear factor NF-kappa-B p65 subunit oligonucleotide, Nucort, Nulojix, Numed-Plus, Nurokind Ortho, Nusone-H, Nutrikemia, Nuvion, NVO7alpha, NX001, Nyclobate, Nyox, Nysa, Obarcort, OC002417, OC2286, ocaratuzumab, OCTSG815, Oedemase, Oedemase-D, ofatumumab, Ofgyl-O, Ofvista, OHR118, OKi, Okifen, Oksamen, Olai, olokizumab, Omeprose E, Omnacortil, Omneed, Omniclor, Omnigel, Omniwel, onercept, ONO4057, ONS1210, ONS1220, Ontac Plus, Ontak, ONX0914, OPC6535, opebacan, OPN101, OPN201, OPN302, OPN305, OPN401, oprelvekin, OPT66, Optifer, Optiflur, OptiMIRA, Orabase Hca, Oradexon, Oraflex, OralFenac, Oralog, Oralpred, Ora-sed, Orasone, orBec, Orbone forte, Orcl, ORE10002, ORE10002, Orencia, Org214007, Org217993, Org219517, Org223119, Org37663, Org39141, Org48762, Org48775, Orgadrone, Ormoxen, Orofen Plus, Oromylase Biogaran, Orthal Forte, Ortho Flex, Orthoclone OKT3, Orthofen, Orthoflam, Orthogesic, Orthoglu, Ortho-II, Orthomac, Ortho-Plus, Ortinims, Ortofen, Orudis, Oruvail, OS2, Oscart, Osmetone, Ospain, Ossilife, Ostelox, Osteluc, Osteocerin, osteopontin, Osteral, otelixizumab, Otipax, Ou Ning, OvaSave, OX40 Ligand Antibody, Oxa, Oxagesic CB, Oxalgin DP, oxaprozin, OXCQ, Oxeno, Oxib MD, Oxibut, Oxicam, Oxiklorin, Oximal, Oxynal, oxyphenbutazone, Oxyphenbutazone, ozoralizumab, P13 peptide, P1639, P21, P2X7 Antagonists, p38 Alpha Inhibitor, p38 Antagonist, p38 MAP kinase inhibitor, p38alpha MAP Kinase Inhibitor, P7 peptide, P7170, P979, PA401, PA517, Pabi-dexamethasone, PAC, PAC10649, paclitaxel, Painoxam, Paldon, Palima, pamapimod, Pamatase, Panafcort, Panafcortelone, Panewin, PanGraf, Panimun Bioral, Panmesone, Panodin SR, Panslay, Panzem, Panzem NCD, PAP1, papain, Papirzin, Pappen K Pap, Paptinim-D, paquinimod, PAR2 Antagonist, Paracetamol, Paradic, Parafen TAJ, Paramidin, Paranac, Parapar, Parci, parecoxib, Parixam, Parry-S, Partaject Busulfan, pateclizumab, Paxceed, PBI0032, PBI1101, PBI1308, PBI1393, PBI1607, PBI1737, PBI2856, PBI4419, PBI4419, P-Cam, PCI31523, PCI32765, PCI34051, PCI45261, PCI45292, PCI45308, PD360324, PD360324, PDA001, PDE4 inhibitor, PDE-IV Inhibitor, PDL241 antibody, PDL252, Pediapred, Pefree, pegacaristim, Peganix, Peg-Interleukin 12, pegsunercept, Pegsunercept, PEGylated arginine deiminase, peldesine, pelubiprofen, Penacle, penicillamine, Penostop, Pentalgin, Pentasa, Pentaud, pentostatin, Peon, Pepdase, Pepser, Peptirase, Pepzen, Pepzol, Percutalgine, Periochip, Peroxisome Proliferator Activated Receptor gamma modulators, Petizene, PF00344600, PF04171327, PF04236921, PF04308515, PF05230905, PF05280586, PF251802, PF3475952, PF3491390, PF3644022, PF4629991, PF4856880, PF5212367, PF5230896, PF547659, PF755616, PF9184, PG27, PG562, PG760564, PG8395, PGE3935199, PGE527667, PHS, PH797804, PHA408, Pharmaniaga Mefenamic acid, Pharmaniaga Meloxicam, Pheldin, Phenocept, phenylbutazone, PHY702, PI3K delta inhibitor, PI3K Gamma/Delta Inhibitor, PI3K Inhibitor, Picalm, pidotimod, piketoprofen, Pilelife, Pilopil, Pilovate, pimecrolimus, Pipethanen, Piractam, Pirexyl, Pirobet, Piroc, Pirocam, Pirofel, Pirogel, Piromed, Pirosol, Pirox, Piroxen, Piroxicam, piroxicam betadex, Piroxifar, Piroxil, Piroxim, Pixim, Pixykine, PKC Theta Inhibitor, PL3100, PL5100 Diclofenac, Placenta Polypeptide, Plaquenil, plerixafor, Plocfen, PLR14, PLR18, Plutin, PLX3397, PLX5622, PLX647, PLX-BMT, pms-Diclofenac, pms-Ibuprofen, pms-Leflunomide, pms-Meloxicam, pms-Piroxicam, pms-Prednisolone, pms-Sulfasalazine, pms-Tiaprofenic, PMX53, PN0615, PN100, PN951, podofilox, POL6326, Polcortolon, Polyderm, Polygam S/D, Polyphlogin, Poncif, Ponstan, Ponstil Forte, Porine-A Neoral, Potaba, potassium aminobenzoate, Potencort, Povidone, povidone iodine, pralnacasan, Prandin, Prebel, Precodil, Precortisyl Forte, Precortyl, Predfoam, Predicort, Predicorten, Predilab, Predilone, Predmetil, Predmix, Predna, Prednesol, Predni, prednicarbate, Prednicort, Prednidib, Prednifarma, Prednilasca, prednisolone, Deltacortril (prednisolone), prednisolone acetate, prednisolone sodium phosphate, prednisolone sodium succinate, prednisolone sodium succinate, prednisone, prednisone acetate, Prednitop, Prednol-L, Prednox, Predone, Predonema, Predsol, Predsolone, Predsone, Predval, Preflam, Prelon, Prenaxol, Prenolone, Preservex, Preservin, Presol, Preson, Prexige, Priliximab, Primacort, Primmuno, Primofenac, prinaberel, Privigen, Prixam, Probuxil, Procarne, Prochymal, Procider-EF, Proctocir, Prodase, Prodel B, Prodent, Prodent Verde, Proepa, Profecom, Profenac L, Profenid, Profenol, Proflam, Proflex, Progesic Z, proglumetacin, proglumetacin maleate, Prograf, Prolase, Prolixan, promethazine hydrochloride, Promostem, Promune, PronaB, pronase, Pronat, Prongs, Pronison, Prontoflam, Propaderm-L, Propodezas, Propolisol, Proponol, propyl nicotinate, Prostaloc, Prostapol, Protacin, Protase, Protease Inhibitors, Protectan, Proteinase Activated Receptor 2 Inhibitor, Protofen, Protrin, Proxalyoc, Proxidol, Proxigel, Proxil, Proxym, Prozym, PRT062070, PRT2607, PRTX100, PRTX200, PRX106, PRX167700, Prysolone, PS031291, PS375179, PS386113, PS540446, PS608504, PS826957, PS873266, Psorid, PT, PT17, PTL101, P-Transfer Factor peptides, PTX3, Pulminiq, Pulsonid, Purazen, Pursin, PVS40200, PX101, PX106491, PX114, PXS2000, PXS2076, PYM60001, Pyralvex, Pyranim, pyrazinobutazone, Pyrenol, Pyricam, Pyrodex, Pyroxi-Kid, QAX576, Qianbobiyan, QPI1002, QR440, qT3, Quiacort, Quidofil, R107s, R125224, R1295, R132811, R1487, R1503, R1524, R1628, R333, R348, R548, R7277, R788, rabeximod, Radix Isatidis, Radofen, Raipeck, Rambazole, Randazima, Rapacan, Rapamune, Raptiva, Ravax, Rayos, RDEA119, RDEA436, RDP58, Reactine, Rebif, REC200, Recartix-DN, receptor for advanced glycation end products antibody, Reclast, Reclofen, recombinant HSA-TIMP-2, recombinant human alkaline Phosphatase, recombinant Interferon Gamma, Recominant human alkaline phosphatase, Reconil, Rectagel HC, Recticin, Recto Menaderm, Rectos, Redipred, Redolet, Refastin, Regenica, REGN88, Relafen, Relaxib, Relev, Relex, Relifen, Relifex, Relitch, Rematof, remestemce1-1, Remesulidum, Remicade® (infliximab), Remsima, Remsima, Remsima, ReN1869, Renacept, Renfor, Renodapt, Renodapt-S, Renta, Reosan, Repare-AR, Reparilexin, reparixin, Repertaxin, Repisprin, Resochin, Resol, resolvin E1, Resurgil, Re-tin-colloid, Retoz, Reumacap, Reumacon, Reumadolor, Reumador, Reumanisal, Reumazin, Reumel, Reumotec, Reuquinol, revamilast, Revascor, Reviroc, Revlimid, Revmoksikam, Rewalk, Rexalgan, RG2077, RG3421, RG4934 antibody, RG7416, RG7624, Rheila, Rheoma, Rheprox, Rheudenolone, Rheufen, Rheugesic, Rheumacid, Rheumacort, Rheumatrex, Rheumesser, Rheumid, Rheumon, Rheumox, Rheuoxib, Rhewlin, Rhucin, RhuDex, Rhulef, Ribox, Ribunal, Ridaura, rifaximin, rilonacept, rimacalib, Rimase, Rimate, Rimatil, Rimesid, risedronate sodium, Ritamine, Rito, Rituxan, rituximab, RNS60, RO1138452, Ro313948, RO3244794, RO5310074, Rob803, Rocamix, Rocas, Rofeb, rofecoxib, Rofee, Rofewal, Roficip Plus, Rojepen, Rokam, Rolodiquim, Romacox Fort, Romatim, romazarit, Ronaben, ronacaleret, Ronoxcin, ROR Gamma T Antagonist, ROR gamma t inverse agonists, Rosecin, rosiglitazone, Rosmarinic acid, Rotan, Rotec, Rothacin, Roxam, Roxib, Roxicam, Roxopro, Roxygin DT, RP54745, RPI78, RPI78M, RPI78MN, RPIMN, RQ00000007, RQ00000008, RTA402, R-Tyflam, Rubicalm, Rubifen, Ruma pap, Rumalef, Rumidol, Rumifen, Runomex, rusalatide acetate, ruxolitinib, RWJ445380, RX10001, Rycloser MR, Rydol, S11³ Receptor Agonists, S11³ Receptor Modulators, S1P1 Agonist, S1P1 receptor agonist, S2474, S3013, SA237, SA6541, Saaz, S-adenosyl-L-methionine-sulfate-p-toluene sulfonate, Sala, Salazidin, Salazine, Salazopyrin, Salcon, Salicam, salsalate, Sameron, SAN300, Sanaven, Sandimmun, Sandoglobulin, Sanexon, SangCya, SAR153191, SAR302503, SAR479746, Sarapep, sargramostim, Sativex, Savantac, Save, Saxizon, Sazo, SB1578, SB210396, SB217969, SB242235, SB273005, SB281832, SB683698, SB751689, SBI087, SC080036, SC12267, SC409, Scaflam, SCD ketoprofen, SCIO323, SCIO469, SD-15, SD281, SDP051 antibody, Sd-rxRNA, secukinumab, Sedase, Sedilax, Sefdene, Seizyme, SEL113, Seladin, Selecox, selectin P ligand antibody, Glucocorticoid Receptor Agonist, Selectofen, Selektine, SelK1 antibody, Seloxx, Selspot, Selzen, Selzenta, Selzentry, semapimod, semapimod hydrochloride, semparatide, Semparatide, Senafen, Sendipen, Senterlic, SEP119249, Sepdase, Septirose, Seractil, Serafen-P, Serase, Seratid D, Seratiopeptidase, Serato-M, Seratoma Forte, Serazyme, Serezon, Sero, Serodase, Serpicam, Serra, serrapeptase, Serratin, Serratiopeptidase, Serrazyme, Servisone, Seven E P, SGI1252, SGN30, SGN70, SGX203, shark cartilage extract, Sheril, Shield, Shifazen, Shifazen-Fort, Shincort, Shincort, Shiosol, ShK186, Shuanghuangxiaoyan, SI615, SI636, Sigmasporin, Sigmasporin, SIM916, Simpone, Simulect, Sinacort, Sinalgia, Sinapol, Sinatrol, Sinsia, siponimod, Sirolim, sirolimus, Siropan, Sirota, Sirova, sirukumab, Sistal Forte, SKF105685, SKF105809, SKF106615, SKF86002, Skinalar, Skynim, Skytrip, SLAM family member 7 antibody, Slo-indo, SM101, SM201 antibody, SM401, SMAD family member 7 oligonucleotide, SMART Anti-IL-12 Antibody, SMP114, SNO030908, SNO070131, sodium aurothiomalate, sodium chondroitin sulfate, sodium deoxyribonucleotide, sodium gualenate, sodium naproxen, sodium salicylate, Sodixen, Sofeo, Soleton, Solhidrol, Solicam, Soliky, Soliris, Sol-Melcort, Solomet, Solondo, Solone, Solu-Cort, Solu-Cortef, Solu-Decortin H, Solufen, Solu-Ket, Solumark, Solu-Medrol, Solupred, Somalgen, somatropin, Sonap, Sone, sonepcizumab, Sonexa, Sonim, Sonim P, Soonil, Soral, Sorenil, sotrastaurin acetate, SP-10, SP600125, Spanidin, SP-Cortil, SPD550, Spedace, sperm adhesion molecule 1, Spictol, spleen tyrosine kinase oligonucleotide, Sporin, S-prin, SPWF1501, SQ641, SQ922, SR318B, SR9025, SRT2104, SSR150106, SSR180575, SSS07 antibody, ST1959, STA5326, stabilin 1 antibody, Stacort, Stalogesic, stanozolol, Staren, Starmelox, Stedex IND-SWIFT, Stelara, Stemin, Stenirol, Sterapred, Steriderm S, Sterio, Sterisone, Steron, stichodactyla helianthus peptide, Stickzenol A, Stiefcortil, Stimulan, STNM01, Store Operated Calcium Channel (SOCC) Modulator, STP432, STP900, Stratasin, Stridimmune, Strigraf, SU Medrol, Subreum, Subuton, Succicort, Succimed, Sulan, Sulcolon, Sulfasalazin Heyl, Sulfasalazin, sulfasalazine, Sulfovit, Sulidac, Sulide, sulindac, Sulindex, Sulinton, Sulphafine, Sumilu, SUN597, Suprafen, Supretic, Supsidine, Surgam, Surgamine, Surugamu, Suspen, Suton, Suvenyl, Suwei, SW Dexasone, Syk Family Kinase Inhibitor, Syn1002, Synacran, Synacthen, Synalar C, Synalar, Synavive, Synercort, Sypresta, T cell cytokine-inducing surface molecule antibody, T cell receptor antibody, T5224, T5226, TA101, TA112, TA383, TA5493, tabalumab, Tacedin, Tacgraf, TACIFc5, Tacrobell, Tacrograf, Tacrol, tacrolimus, Tadekinig alpha, Tadolak, TAFA93, Tafirol Artro, Taizen, TAK603, TAK715, TAK783, Takfa, Taksta, talarozole, Talfin, Talmain, talmapimod, Talmea, Talnif, talniflumate, Talos, Talpain, Talumat, Tamalgen, Tamceton, Tamezon, Tandrilax, tannins, Tannosynt, Tantum, tanzisertib, Tapain-beta, Tapoein, Tarenac, tarenflurbil, Tarimus, Tarproxen, Tauxib, Tazomust, TBR652, TC5619, T-cell, immune regulator 1, ATPase, H+ transporting, lysosomal VO subunit A3 antibody, TCK1, T-cort, T-Dexa, Tecelac, Tecon, teduglutide, Teecort, Tegeline, Tementil, temoporfin, Tencam, Tendrone, Tenefuse, Tenfly, tenidap sodium, Tenocam, Tenoflex, Tenoksan, Tenotil, tenoxicam, Tenoxim, Tepadina, Teracort, Teradol, tetomilast, TG0054, TG1060, TG20, TG20, tgAAC94, Th1/Th2 Cytokine Synthase Inhibitor, Th-17 cell inhibitors, Thalido, thalidomide, Thalomid, Themisera, Thenil, Therafectin, Therapyace, thiarabine, Thiazolopyrimidines, thioctic acid, thiotepa, THR090717, THR0921, Threenofen, Thrombate III, Thymic peptide, Thymodepressin, Thymogam, Thymoglobulin, Thymoglobuline, Thymoject thymic peptides, thymomodulin, thymopentin, thymopolypetides, tiaprofenic acid, tibezonium iodide, Ticoflex, tilmacoxib, Tilur, T-immune, Timocon, Tiorase, Tissop, TKB662, TL011, TLR4 antagonists, TLR8 inhibitor, TM120, TM400, TMX302, TNF Alpha inhibitor, TNF alpha-TNF receptor antagonist, TNF antibody, TNF receptor superfamily antagonists, TNF TWEAK Bi-Specific, TNF-Kinoid, TNFQb, TNFR1 antagonist, TNR001, TNX100, TNX224, TNX336, TNX558, tocilizumab, tofacitinib, Tokuhon happ, TOL101, TOL102, Tolectin, ToleriMab, Tolerostem, Tolindol, toll-like receptor 4 antibody, toll-like receptor antibody, tolmetin sodium, Tongkeeper, Tonmex, Topflame, Topicort, Topleucon, Topnac, Toppin Ichthammol, toralizumab, Toraren, Torcoxia, Toroxx, Tory, Toselac, Totaryl, Touch-med, Touchron, Tovok, Toxic apis, Toyolyzom, TP4179, TPCA1, TPI526, TR14035, Tradil Fort, Traficet-EN, Tramace, tramadol hydrochloride, tranilast, Transimune, Transporina, Tratul, Trexall, Triacort, Triakort, Trialon, Triam, triamcinolone, triamcinolone acetate, triamcinolone acetonide, triamcinolone acetonide acetate, triamcinolone hexacetonide, Triamcort, Triamsicort, Trianex, Tricin, Tricort, Tricortone, TricOs T, Triderm, Trilac, Trilisate, Trinocort, Trinolone, Triolex, triptolide, Trisfen, Trivaris, TRK170, TRK530, Trocade, trolamine salicylate, Trolovol, Trosera, Trosera D, Troycort, TRX1 antibody, TRX4, Trymoto, Trymoto-A, TT301, TT302, TT32, TT32, TT33, TTI314, tumor necrosis factor, tumor necrosis factor 2-methoxyethyl phosphorothioate oligonucleotide, tumor necrosis factor antibody, tumor necrosis factor kinoid, tumor necrosis factor oligonucleotide, tumor necrosis factor receptor superfamily, member 1B antibody, tumor necrosis factor receptor superfamilylB oligonucleotide, tumor necrosis factor superfamily, member 12 antibody, tumor necrosis factor superfamily, member 4 antibody, tumor protein p53 oligonucleotide, tumour necrosis factor alpha antibody, TuNEX, TXA127, TX-RAD, TYK2 inhibitors, Tysabri, ubidecarenone, Ucerase, ulodesine, Ultiflam, Ultrafastin, Ultrafen, Ultralan, U-Nice-B, Uniplus, Unitrexate, Unizen, Uphaxicam, UR13870, UR5269, UR67767, Uremol-HC, Urigon, U-Ritis, ustekinumab, V85546, Valcib, Valcox, valdecoxib, Valdez, Valdixx, Valdy, Valentac, Valoxib, Valtune, Valus AT, Valz, Valzer, Vamid, Vantal, Vantelin, VAP-1 SSAO Inhibitor, vapaliximab, varespladib methyl, Varicosin, Varidase, vascular adhesion protein-1 antibody, VB110, VB120, VB201, VBY285, Vectra-P, vedolizumab, Vefren, VEGFR-1 Antibody, Veldona, veltuzumab, Vendexine, Venimmun N, Venoforte, Venoglobulin-IH, Venozel, Veral, Verax, vercirnon, vero-dexamethasone, Vero-Kladribin, Vetazone, VGX1027, VGX750, Vibex MTX, vidofludimus, Vifenac, Vimovo, Vimultisa, Vincort, Vingraf, Vioform-HC, Vioxl, Vioxx, Virobron, visilizumab, Vivaglobin, Vivalde Plus, Vivian-A, VLST002, VLST003, VLST004, VLST005, VLST007, Voalla, voclosporin, Vokam, Vokmor, Volmax, Volna-K, Voltadol, Voltagesic, Voltanase, Voltanec, Voltaren, Voltarile, Voltic, Voren, vorsetuzumab, Votan-SR, VR909, VRA002, VRP1008, VRS826, VRS826, VT111, VT214, VT224, VT310, VT346, VT362, VTX763, Vurdon, VX30 antibody, VX467, VXS, VX509, VX702, VX740, VX745, VX745, VX850, W54011, Walacort, Walix, WC3027, Wilgraf, Winflam, Winmol, Winpred, Winsolve, Wintogeno, WIP901, Woncox, WSB711 antibody, WSB712 antibody, WSB735, WSB961, X071NAB, X083NAB, Xantomicin Forte, Xedenol, Xefo, Xefocam, Xenar, Xepol, X-Flam, Xibra, Xicam, Xicotil, Xifaxan, XL499, XmAb5483, XmAb5485, XmAb5574, XmAb5871, XOMA052, Xpress, XPro1595, XtendTNF, XToll, Xtra, Xylex-H, Xynofen SR, Yang Shu-IVIG, YHB14112, YM974, Youfeline, Youfenac, Yuma, Yumerol, Yuroben, YY piroxicam, Z104657A, Zacy, Zaltokin, zaltoprofen, Zap70 Inhibitor, Zeepain, Zeloxim Fort, Zema-Pak, Zempack, Zempred, Zenapax, Zenas, Zenol, Zenos, Zenoxone, Zerax, Zerocam, Zerospasm, ZFNs, zinc oxide, Zipsor, ziralimumab, Zitis, Zix-S, Zocort, Zodixam, Zoftadex, zoledronic acid, Zolfin, Zolterol, Zopyrin, Zoralone, ZORprin, Zortress, ZP1848, zucapsaicin, Zunovate, Zwitterionic polysaccharides, ZY1400, Zybodies, Zycel, Zyrofen, Zyrogen Inhibitors, Zyser, Zytrim, and Zywin-Forte. In addition, the anti-inflammatory drugs, as listed above, may be combined with one or more agents listed above or herein or with other agents known in the art.

In one embodiment, the drug is a drug that inhibits, reduces or modulates the signaling and/or activity of PDGF-receptors (PDGFR). For example, the PDGF antagonist delivered to the suprachoroidal space for the treatment of one or more posterior ocular disorders such as macular edema associated with uveitis (e.g., non-infectious uveitis), macular edema associated with RVO or wet AMD, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFRα or PDGFRβ. In one embodiment, the PDGF antagonist is the anti-PDGF-β aptamer E10030, dasatinib, sunitinib, axitinib, sorefenib, imatinib, imatinib mesylate, nintedanib, pazopanib HCl, ponatinib , MK-2461, pazopanib, crenolanib, PP-121, telatinib, imatinib, KRN 633, CP 673451, TSU-68 (orantinib), Ki8751, amuvatinib, tivozanib, masitinib, motesanib diphosphate, dovitinib, dovitinib dilactic acid, FOVISTA, or linifanib (ABT-869). As described herein, in one embodimdent, the PDGF antagonist, for example, one of the PDGF antagonists described above, can be used in the methods for treating macular edema associated with uveitis, macular edema associated with RVO or wet AMD via SCS administration. Moreover, in some embodiments, the PDGF antagonist is administered intravitreally in conjunction with SCS administration of an anti-inflammatory agent, in a method of treating macular edema associated with RVO.

In a further embodiment, the PDGF antagonist also has VEGF antagonist activity. For example, an anti-VEGF/PDGF-B darpin, dasatinib, dovitinib, Ki8751, telatinib, TSU-68 (orantinib) or motesanib diphosphate are known inhibitors of both VEGF and PDGF, and can be used in the methods described herein, for example, for the treatment of macular edema associated with uveitis, macular edema associated with RVO or wet AMD. The dual PDGF/VEGF antagonist can also be administered intravitreally in conjunction with non-surgical delivery of an anti-inflammatory compound to the SCS in a method of treating macular emeda associated with RVO.

Examples of other suitable drugs for use with the devices and methods described herein include, but are not limited to: A0003, A36 peptide, AAV2-sFLT01, ACE041, ACU02, ACU3223, ACU4429, AdPEDF, aflibercept, AG13958, aganirsen, AGN150998, AGN745, AL39324, AL78898A, AL8309B, ALN-VEG01, alprostadil, AM1101, amyloid beta antibody, anecortave acetate, Anti-VEGFR-2 Alterase, Aptocine, APX003, ARC1905, ARC1905 with Lucentis, ATG3, ATP-binding cassette, sub-family A, member 4 gene, ATXS10, Avastin with Visudyne, AVT101, AVT2, bertilimumab, bevacizumab with verteporfin, bevasiranib sodium, bevasiranib sodium; with ranibizumab, brimonidine tartrate, BVA301, canakinumab, Cand5, Cand5 with Lucentis, CERE140, ciliary neurotrophic factor, CLT009, CNT02476, collagen monoclonal antibody, complement component 5 aptamer (pegylated), complement component 5 aptamer (pegylated) with ranibizumab, complement component C3, complement factor B antibody, complement factor D antibody, copper oxide with lutein, vitamin C, vitamin E, and zinc oxide, dalantercept, DE109, bevacizumab, ranibizumab, triamcinolone, triamcinolone acetonide, triamcinolone acetonide with verteporfin, dexamethasone, dexamethasone with ranibizumab and verteporfin, disitertide, DNA damage inducible transcript 4 oligonucleotide, E10030, E10030 with Lucentis, EC400, eculizumab, EGP, EHT204, embryonic stem cells, human stem cells, endoglin monoclonal antibody, EphB4 RTK Inhibitor, EphB4 Soluble Receptor, ESBA1008, ETX6991, Evizon, Eyebar, EyePromise Five, Eyevi, Eylea, F200, FCFD4514S, fenretinide, fluocinolone acetonide, fluocinolone acetonide with ranibizumab, fms-related tyrosine kinase 1 oligonucleotide, fms-related tyrosine kinase 1 oligonucleotide with kinase insert domain receptor 169, fosbretabulin tromethamine, Gamunex, GEM220, GS101, GSK933776, HC31496, Human n-CoDeR, HYB676, IBI-20089 with ranibizumab (Lucentis®), iCo-008, Icon1, I-Gold, Ilaris, Iluvien, Iluvien with Lucentis, immunoglobulins, integrin alpha5beta1 immunoglobulin fragments, Integrin inhibitor, IRIS Lutein, I-Sense Ocushield, Isonep, isopropyl unoprostone, JPE1375, JSM6427, KH902, LentiVue, LFG316, LP590, LP01010AM, Lucentis, Lucentis with Visudyne, Lutein ekstra, Lutein with myrtillus extract, Lutein with zeaxanthin, M200, M200 with Lucentis, Macugen, MC1101, MCT355, mecamylamine, Microplasmin, motexafin lutetium, MP0112, NADPH oxidase inhibitors, aeterna shark cartilage extract (Arthrovas™, Neoretna™, Psovascar™), neurotrophin 4 gene, Nova21012, Nova21013, NT501, NT503, Nutri-Stulln, ocriplasmin, OcuXan, Oftan Macula, Optrin, ORA102 with bevacizumab (Avastin®), P144, P17, Palomid 529, PAN90806, Panzem, Panzem, PARP inhibitors, pazopanib hydrochloride, pegaptanib sodium, PF4523655, PG11047, piribedil, platelet-derived growth factor beta polypeptide aptamer (pegylated), platelet-derived growth factor beta polypeptide aptamer (pegylated) with ranibizumab, PLG101, PMX20005, PMX53, POT4, PRS055, PTK787, ranibizumab, ranibizumab with triamcinolone acetonide, ranibizumabwith verteporfin, ranibizumab with volociximab, RD27, Rescula, Retaane, retinal pigment epithelial cells, RetinoStat, RG7417, RN6G, RT101, RTU007, SB267268, serpin peptidase inhibitor, Glade F, member 1 gene, shark cartilage extract, Shef1, SIR1046, SIR1076, Sirna027, sirolimus, SMTD004, Snelvit, SOD Mimetics, Soliris, sonepcizumab, squalamine lactate, ST602, StarGen, T2TrpRS, TA106, talaporfin sodium, Tauroursodeoxycholic acid , TG100801, TKI , TLCx99, TRC093, TRC105, Trivastal Retard, TT30, Ursa, ursodiol, Vangiolux, VAR10200, vascular endothelial growth factor antibody, vascular endothelial growth factor B, vascular endothelial growth factor kinoid, vascular endothelial growth factor oligonucleotide, VAST Compounds, vatalanib, VEGF antagonist (e.g., as described herein), verteporfin, Visudyne, Visudyne with Lucentis and dexamethasone, Visudyne with triamcinolone acetonide, Vivis, volociximab, Votrient, XV615, zeaxanthin, ZFP TF, zinc-monocysteine and Zybrestat. In one embodiment, one or more of the drugs described above is combined with one or more agents listed above or herein or with other agents known in the art.

In one embodiment, the drug is interferon gamma 1b (Actimmune®) with pirfenidone, ACUHTR028, AlphaVBeta5, aminobenzoate potassium, amyloid P, ANG1122, ANG1170, ANG3062, ANG3281, ANG3298, ANG4011, Anti-CTGF RNAi, Aplidin, astragalus membranaceus extract with salvia and schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, Galectin-3 inhibitors, GKT137831, GMCT01, GMCT02, GRMD01, GRMD02, GRN510, Heberon Alfa R, interferon alfa-2b, interferon gamma-1b with pirfenidone, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 Fusion Proteins, RXI109, secretin, STX100, TGF-beta Inhibitor, transforming growth factor, beta receptor 2 oligonucleotide, VA999260 or XV615. In one embodiment, one or more of the drugs for treating macular edema associated with uveitis described above is combined with one or more agents listed above or herein or with other agents known in the art.

In one embodiment, a drug that treats, prevents and/or ameliorates diabetic macular edema is used in conjunction with the devices and methods described herein and is delivered to the suprachoroidal space of the eye. In a further embodiment, the drug is AKB9778, bevasiranib sodium, Candy, choline fenofibrate, Cortiject, c-raf 2-methoxyethyl phosphorothioate oligonucleotide, DE109, dexamethasone, DNA damage inducible transcript 4 oligonucleotide, FOV2304, iCo007, KH902, MP0112, NCX434, Optina, Ozurdex, PF4523655, SAR1118, sirolimus, SK0503 or TriLipix. In one embodiment, one or more of the diabetic macular edema treating drugs described above is combined with one or more agents listed above or herein or with other agents known in the art.

In one embodiment, the methods and devices provided herein are used to deliver triamcinolone or triamcinolone acetonide to the suprachoroidal space of an eye of a human subject in need of treatment for treating uveitis (e.g., non-infectious uveitis), macular edema associated with uveitis, macular edema associated with RVO, or wet AMD. In another embodiment, triamcinolone or triamcinolone acetonide is delivered via one of the methods described herein.

The triamcinolone composition provided herein, in one embodiment, is a suspension comprising microparticles or nanoparticles of triamcinolone or triamcinolone acetonide. The microparticles, in one embodiment, have a D₅₀ of about 3 μm or less. In a further embodiment, the D₅₀ is about 2 μm. In another embodiment, the D₅₀ is about 2 μm or less. In even another embodiment, the D₅₀ is about 1000 nm or less. The microparticles, in one embodiment, have a D₉₉ of about 10 μm or less. In another embodiment, the D₉₉ is about 10 μm. In another embodiment, the D₉₉ is about 10 μm or less, or about 9 μm or less.

In one embodiment, triamcinolone is present in the composition at from about 1 mg/mL to about 400 mg/mL. In a further embodiment, triamcinolone is present in the composition at from about 2 mg/mL to about 300 mg/mL. In a further embodiment, triamcinolone is present in the composition at from about 5 mg/mL to about 200 mg/mL. In a further embodiment, triamcinolone is present in the composition at from about 10 mg/mL to about 100 mg/mL. In a further embodiment, triamcinolone is present in the composition at from about 20 mg/mL to about 75 mg/mL. In a further embodiment, triamcinolone is present in the composition at from about 30 mg/mL to about 50 mg/mL. In one embodiment, triamcinolone is present in the composition at about 10, about 20, about 25, about 30, about 35, about 40, about 45, about 50, about 55 about 60, or about 75 mg/mL. In one embodiment, triamcinolone is present in the composition at about 40 mg/mL.

In one embodiment, the triamcinolone composition comprises sodium chloride. In another embodiment, the triamcinolone composition comprises carboxymethylcellulose sodium.

In one embodiment, the triamcinolone composition comprises triamcinolone microparticles. In a further embodiment, the composition comprises polysorbate 80. In another embodiment, the triamcinolone composition comprises one or more of CaCl₂, MgCl₂, sodium acetate and sodium citrate. In one embodiment, the composition comprises polysorbate 80 at a w/v % of 0.02% or about 0.02%, 0.015% or about 0.015%.

In one embodiment, the pH of the composition is from about 5.0 to about 8.5. In a further embodiment, the pH of the composition is from about 5.5 to about 8.0. In a yet further embodiment, the pH of the composition os from about 6.0 to about 7.5.

In one embodiment, the therapeutic formulation comprises a suspension of cells, for example, a suspension of rentinal stem cells. In one embodiment, a suspension of neural stem cells (NSCs) is administered to the SCS via one of the devices and/or methods provided herien. NSCs are self-renewing, multipotent cells that can differentiate into the main cell phenotypes of the nervous system. They have been isolated from the adult mammalian brain tissue, including humans. In one embodiment, a suspension of retinal stem cells (RSCs) is administered to the SCS via one of the devices and/or methods provided herien. During early development, retinal stem cells (RSC) are a possible donor source that give rise to all retinal cell types. These cells can be isolated, expanded, and differentiated into retinal neurons by culturing them in the presence of growth factors, such as epidermal growth factor and fibroblast growth factor. In yet another embodiment, a suspension of adult stem cells or mesenchymal stem cells (MSCs) is administered to the SCS of a patient in need thereof via one of the devices and/or methods provided herein. Other cell types amenable for administration via the devices and methods provided herein include but are not limited to hematopoietic stem cells (HSCs), human embryonic stem cells (hESCs), retinal progenitor cells, endothelial progenitor cells or a combination thereof.

In one embodiment, one or more of the stem cells described in Arch Ophthalmol. 2004;122(4):621-627, incorporated by reference herein in its entirety for all purposes, is delivered to a patient via a device or method described herein.

The “therapeutic formulation” delivered via the methods and devices provided herein in one embodiment, is an aqueous solution or suspension, and comprises an effective amount of the drug or therapeutic agent, for example, a cellular suspension. In some embodiments, the therapeutic formulation is a fluid drug formulation. The “drug formulation” is a formulation of a drug, which typically includes one or more pharmaceutically acceptable excipient materials known in the art. The term “excipient” refers to any non-active ingredient of the formulation intended to facilitate handling, stability, dispersibility, wettability, release kinetics, and/or injection of the drug. In one embodiment, the excipient may include or consist of water or saline.

The therapeutic formulation delivered to the suprachoroidal space of the eye of a human subject for the treatment of uveitis (e.g., non-infectious uveitis), macular edema associated with uveitis (e.g., non-infectious uveitis), macular edema associated with RVO or wet AMD, may be in the form of a liquid drug, a liquid solution that includes a drug or therapy in a suitable solvent, or liquid suspension. The liquid suspension may include microparticles or nanoparticles dispersed in a suitable liquid vehicle for infusion. In various embodiments, the drug is included in a liquid vehicle, in microparticles or nanoparticles, or in both the vehicle and particles. The drug formulation is sufficiently fluid to flow into and within the suprachoroidal space, as well as into the surrounding posterior ocular tissues. In one embodiment, the viscosity of the fluid drug formulation is about 1 cP at 37° C.

In one embodiment, the drug formulation (e.g., fluid drug formulation) includes microparticles or nanoparticles, either of which includes at least one drug. Desirably, the microparticles or nanoparticles provide for the controlled release of drug into the suprachoroidal space and surrounding posterior ocular tissue. As used herein, the term “microparticle” encompasses microspheres, microcapsules, microparticles, and beads, having a number average diameter of from about 1 μm to about 100 μm, for example from about 1 to about 25 μm, or from about 1 μm to about 7 μm. “Nanoparticles” are particles having an average diameter of from about 1 nm to about 1000 nm. The microparticles, in one embodiment, have a D₅₀ of about 3 μm or less. In a further embodiment, the D₅₀ is about 2 μm. In another embodiment, the D₅₀ of the particles in the drug formulation is about 2 μm or less. In another embodiment, the D₅₀ of the particles in the drug formulation is about 1000 nm or less. In one embodiment, the drug formulation comprises microparticles having a D₉₉ of about 10 μm or less. The microparticles, in one embodiment, have a D₅₀ of about 3 μm or less. In a further embodiment, the D₅₀ is about 2 μm. In another embodiment, the D₅₀ of the particles in the drug formulation is about 2 μm or less. In another embodiment, the D50 of the particles in the drug formulation is about 1000 nm or less. In one embodiment, the drug formulation comprises microparticles having a D₉₉ of about 10 μm or less. The microparticles, in one embodiment, have a D₅₀ of about 3 μm or less. In a further embodiment, the D₅₀ is about 2 μm. In another embodiment, the D₅₀ of the particles in the drug formulation is about 2 μm or less. In another embodiment, the D₅₀ of the particles in the drug formulation is about 100 nm to about 1000 nm. In one embodiment, the drug formulation comprises microparticles having a D₉₉ of about 1000 nm to about 10 um. The microparticles, in one embodiment, have a D₅₀ of about 1 μm to about 5 μm or less. In another embodiment, the drug formulation comprises particles having a D₉₉ of about 10 μm. In another embodiment, the D₉₉ of the particles in the formulation is less than about 10 μm, or less than about 9 μm, or less than about 7 μm or less than about 3 μm. In a further embodiment, the microparticles or nanoparticles comprise an anti-inflammatory drug. In a further embodiment, the anti-inflammatory drug is triamcinolone.

Microparticles and nanoparticles may or may not be spherical in shape. “Microcapsules” and “nanocapsules” are defined as microparticles and nanoparticles having an outer shell surrounding a core of another material. The core can be liquid, gel, solid, gas, or a combination thereof. In one case, the microcapsule or nanocapsule may be a “microbubble” or “nanobubble” having an outer shell surrounding a core of gas, wherein the drug is disposed on the surface of the outer shell, in the outer shell itself, or in the core. (Microbubbles and nanobubles may be respond to accoustic vibrations as known in the art for diagnosis or to burst the microbubble to release its payload at/into a select ocular tissue site.) “Microspheres” and “nanospheres” can be solid spheres, can be porous and include a sponge-like or honeycomb structure formed by pores or voids in a matrix material or shell, or can include multiple discrete voids in a matrix material or shell. The microparticles or nanoparticles may further include a matrix material. The shell or matrix material may be a polymer, amino acid, saccharride, or other material known in the art of microencapsulation.

The drug-containing microparticles or nanoparticles may be suspended in an aqueous or non-aqueous liquid vehicle. The liquid vehicle may be a pharmaceutically acceptable aqueous solution, and optionally may further include a surfactant. The microparticles or nanoparticles of drug themselves may include an excipient material, such as a polymer, a polysaccharide, a surfactant, etc., which are known in the art to control the kinetics of drug release from particles.

In one embodiment, the drug formulation further includes an agent effective to degrade collagen or GAG fibers in the sclera, which may enhance penetration/release of the drug into the ocular tissues. This agent may be, for example, an enzyme, such a hyaluronidase, a collagenase, or a combination thereof. In a variation of this method, the enzyme is administered to the ocular tissue in a separate step from—preceding or following—infusion of the drug. The enzyme and drug are administered at the same site.

In another embodiment, the drug formulation is one which undergoes a phase change upon administration. For instance, a liquid drug formulation may be injected through hollow microneedles into the suprachoroidal space, where it then gels and the drug diffuses out from the gel for controlled release.

The therapeutic substance in one embodiment is formulated with one or more polymeric excipients to limit therapeutic substance migration and/or to increase viscosity of the formulation. A polymeric excipient may be selected and formulated to act as a viscous gel-like material in-situ and thereby spread into a region of the suprachoroidal space and uniformly distribute and retain the drug. The polymer excipient in one embodiment is selected and formulated to provide the appropriate viscosity, flow and dissolution properties. For example, carboxymethylcellulose is used in one embodiment to form a gel-like material in the suprachoroidal space. The viscosity of the polymer in one embodiment is enhanced by appropriate chemical modification to the polymer to increase associative properties such as the addition of hydrophobic moieties, the selection of higher molecular weight polymer or by formulation with appropriate surfactants.

The dissolution properties of the therapeutic formulation in one embodiment is adjusted by tailoring of the water solubility, molecular weight, and concentration of the polymeric excipient in the range of appropriate thixotropic properties to allow both delivery through a small gauge needle and localization in the suprachoroidal space. The polymeric excipient may be formulated to increase in viscosity or to cross-link after delivery to further limit migration or dissolution of the material and incorporated drug.

Water soluble polymers that are physiologically compatible are suitable for use as polymeric excipients in the therapeutic formulations described herein, and for delivery via the methods and devices described herein include but are not limited to synthetic polymers such as polyvinylalcohol, polyvinylpyrollidone, polyethylene glycol, polyethylene oxide, polyhydroxyethylmethacrylate, polypropylene glycol and propylene oxide, and biological polymers such as cellulose derivatives, chitin derivatives, alginate, gelatin, starch derivatives, hyaluronic acid, chondroiten sulfate, dermatin sulfate, and other glycosoaminoglycans, and mixtures or copolymers of such polymers. The polymeric excipient is selected in one embodiment to allow dissolution over time, with the rate controlled by the concentration, molecular weight, water solubility, crosslinking, enzyme lability and tissue adhesive properties of the polymer.

In one embodiment, a viscosity modifying agent is present in a therapeutic formulation delivered by one of the methods and/or devices described herein. In a further embodiment, the viscosity modifying agent is polyvinyl alcohol, polyvinyl pyrrolidone, methyl cellulose, hydroxypropyl cellulose, hydroxyethyl cellulose, hydroxymethyl cellulose or hydroxypropyl cellulose. In another embodiment, the formulation comprises a gelling agent such as poly(hydroxymethylmethacrylate), poly(N-vinylpyrrolidone), polyvinyl alcohol or an acrylic acid polymer such as Carbopol.

In one embodiment, the therapeutic formulation is delivered via one of the methods and or devices described herein as a liposomal formulation.

Liposomes can be produced by a variety of methods. Bangham's procedure (J. Mol. Biol., J Mol Biol. 13(1):238-52, 1965) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation. Each of the patents references in this paragraph is incorporated by reference herein in their entireties for all purposes.

In one embodiment, the liposomal formulation comprises a phosolipid. In a further embodiment, the liposomal formulation comprises a sterol such as cholesterol.

In another embodiment, the liposomal formulation comprises unilamellar vesiscles. Unilamellar vesicles can be produced from MLVs by a number of techniques, for example, the extrusion of Cullis et al. (U.S. Pat. No. 5,008,050) and Loughrey et al. (U.S. Pat. No. 5,059,421). Sonication and homogenization can be used to produce smaller unilamellar liposomes from larger liposomes (see, for example, Paphadjopoulos et al., Biochim. Biophys. Acta., 135:624-638, 1967; Deamer, U.S. Pat. No. 4,515,736; and Chapman et al., Liposome Technol., 1984, pp. 1-18). A review of these and other methods for producing liposomes can be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467). Each of the references in this paragraph is incorporated by reference herein in their entireties for all purposes.

As described above, the drug formulation delivered to the suprachoroidal space via the methods described herein, e.g., for treating macular edema associated with uveitis or macular edema associated with RVO, can be administered with one or more additional drugs. The one or more additional drugs, in one embodiment, are present in the same formulation as the initial drug formulation. In another embodiment, the one or more additional drugs are present in a second formulation. In even a further embodiment, the second drug formulation is delivered to the patient in need thereof via a non-surgical SCS delivery method described herein. Alternatively, the second drug formulation is delivered intravitreally, intracamerally, sub-tenonally, orally, topically or parenterally to the human subject in need of treatment of macular edema associated with uveitis or macular edema associated with RVO. In one embodiment, a VEGF antagonist is delivered to the suprachoroidal space of the eye of a human subject in need of treatment of macular edema associated with uveitis or macular edema associated with RVO via one of the methods and/or devices disclosed herein, in conjunction with an anti-inflammatory compound.

As described above, in addition to suprachoroidal delivery, the one or more additional drugs delivered to the human subject can be delivered via intravitreal (IVT) administration (e.g., intravitreal injection, intravitreal implant or eye drops). Methods of IVT administration are well known in the art. Examples of classes of drugs that can be administered via IVT include, but are not limited to: VEGF modulators, PDGF modulators, anti-inflammatory drugs. Examples of drugs that can be administered via IVT include, but are not limited to: A0003, A0006, Acedolone, AdPEDF, aflibercept, AG13958, aganirsen, AGN208397, AKB9778, AL78898A, amyloid P, Angiogenesis Inhibitor Gene Therapy, ARC1905, Aurocort, bevasiranib sodium, brimonidine, Brimonidine, brimonidine tartrate, bromfenac sodium, Candy, CERE140, Ciganclor, CLT001, CLT003, CLT004, CLT005, complement component 5 aptamer (pegylated), complement factor D antibody, Cortiject, c-raf 2-methoxyethyl phosphorothioate oligonucleotide, cyclosporine, triamcinolone, DE109, denufosol tetrasodium, dexamethasone, dexamethasone phosphate, disitertide, DNA damage inducible transcript 4 oligonucleotide, E10030, ecallantide, EG3306, Eos013, ESBA1008, ESBA105, Eylea, FCFD4514S, fluocinolone acetonide, fms-related tyrosine kinase 1 oligonucleotide, fomivirsen sodium, fosbretabulin tromethamine, FOV2301, FOV2501, ganciclovir, ganciclovir sodium, GS101, GS156, hyaluronidase, IBI20089, iCo007, Iluvien, INS37217, Isonep, JSM6427, Kalbitor, KH902, lerdelimumab, LFG316, Lucentis®, M200, Macugen, Makyueido, Microplasmin, MK0140, MP0112, NCX434, neurotrophin 4 gene, OC10X, ocriplasmin, ORA102, Ozurdex, P144, P17, Palomid 529, pazopanib hydrochloride, pegaptanib sodium, Plasma Kallikrein Inhibitors, platelet-derived growth factor beta polypeptide aptamer (pegylated), POT4, PRM167, PRS055, QPI1007, ranibizumab, resveratrol, Retilone, retinal pigment epithelium-specific protein 65 kDa gene, Retisert, rod derived cone viability factor, RPE65 Gene Therapy, RPGR Gene Therapy, RTP801, Sd-rxRNA, serpin peptidase inhibitor Glade F member 1 gene, Sirna027, sirolimus, sonepcizumab, SRT501, STP601, TG100948, Trabio, triamcinolone, triamcinolone acetonide, Trivaris, tumor necrosis factor antibody, VEGF/rGel-Op, verteporfin, Visudyne, Vitrase, Vitrasert, Vitravene, Vitreals, volociximab, Votrient, XG102, Xibrom, XV615, and Zybrestat. Accordingly, the methods of the present invention include administrating via IVT one or more of the drugs listed above in combination with one or more drugs disclosed herein administered into the suprachoroidal space using the microneedle device described herein.

### EXAMPLES

The present invention is further illustrated by reference to the following Examples. However, it should be noted that these Examples, like the embodiments described above, are illustrative and are not to be construed as restricting the scope of the invention in any way.

### Example 1

### Triamcinolone Formulations for Delivery to the Suprachoroidal Space

Triamcinolone is delivered to the suprachoroidal space using the methods and devices provided herein. The triamcinolone formulation, in one embodiment, is selected from one of the following seven formulations in Table 2.

TABLE 2
Triamcinolone (TA) formulations.

| Ingredient | Formulation A | | Formulation B | | Formulation C | | Formulation D | |
|---|---|---|---|---|---|---|---|---|
| Triamcinolone | 40 | mg/mL | 40 | mg/mL | 40 | mg/mL | 40 | mg/mL |
| acetonide | | | | | | | | |
| Particle | D₅₀: ~2 | μm | D₅₀: ~2 | μm | D₅₀: ~2 | μm | D₅₀: ~2 | μm |
| Size | D₉₉: <10 | μm | D₉₉: <10 | μm | D₉₉: <10 | μm | D₉₉: <10 | μm |
| Sodium | 0.64% | w/v | 0.64% | w/v | 0.64% | w/v | 0.55% | w/v |
| Chloride | | | | | | | | |
| Carboxymethylcellulose | 0.5% | w/v | 0.5% | w/v | 0.5% | w/v | 0.5% | w/v |
| sodium | | | | | | | | |
| Polysorbate | 0.02% | w/v | 0.015% | w/v | ≧0.015% | w/v | 0.015% | w/v |
| 80 | | | | | | | | |
| KCl | 0.075% | w/v | 0.075% | w/v | 0.075% | w/v | 0.075% | w/v |
| CaCl₂ | 0.048% | w/v | 0.048% | w/v | 0.048% | w/v | 0.048% | w/v |
| (dihydrate) | | | | | | | | |
| MgCl₂ | 0.030% | w/v | 0.030% | w/v | 0.030% | w/v | 0.030% | w/v |
| (hexahydrate) | | | | | | | | |
| Sodium | 0.39% | w/v | 0.39% | w/v | 0.39% | w/v | 0.39% | w/v |
| acetate | | | | | | | | |
| (trihydrate) | | | | | | | | |
| Sodium | 0.17% | w/v | 0.17% | w/v | 0.17% | w/v | 0.17% | w/v |
| citrate | | | | | | | | |
| (dihydrate) | | | | | | | | |

| | | | | |
|---|---|---|---|---|
| NaOH/HCl | Adjust to | Adjust to | Adjust to | Adjust to |
| | pH 6.0-7.5 | pH 6.0-7.5 | pH 6.0-7.5 | pH 6.0-7.5 |

| Ingredient | Formulation E | | Formulation F | | Formulation G | |
|---|---|---|---|---|---|---|
| Triamcinolone | 40 | mg/mL | 40 | mg/mL | 8 | mg/mL |
| acetonide | | | | | | |
| Particle | D₅₀: ~2 | μm | D₅₀: ~2 | μm | D₅₀: ~2 | μm |
| Size | D₉₉: <10 | μm | D₉₉: <10 | μm | D₉₉: <10 | μm |
| Sodium | 0.64% | w/v | 0.55% | w/v | 0.55% | w/v |
| Chloride | | | | | | |
| Carboxymethylcellulose | 0.5% | w/v | 0.5% | w/v | 0.5% | w/v |
| sodium | | | | | | |
| Polysorbate | 0.02% | w/v | 0.02% | w/v | 0.01% | w/v |
| 80 | | | | | | |
| KCl | 0.075% | w/v | 0.075% | w/v | 0.075% | w/v |
| CaCl₂ | 0.048% | w/v | 0.048% | w/v | 0.048% | w/v |
| (dihydrate) | | | | | | |
| MgCl₂ | 0.030% | w/v | 0.030% | w/v | 0.030% | w/v |
| (hexahydrate) | | | | | | |
| Sodium | 0.39% | w/v | 0.39% | w/v | 0.39% | w/v |
| acetate | | | | | | |
| (trihydrate) | | | | | | |
| Sodium | 0.17% | w/v | 0.17% | w/v | 0.17% | w/v |
| citrate | | | | | | |
| (dihydrate) | | | | | | |

| | | | |
|---|---|---|---|
| NaOH/HCl | Adjust to | Adjust to | Adjust to |
| | pH 6.0-7.5 | pH 6.0-7.5 | pH 6.0-7.5 |

### Example 2

### Phase 1/2 Open-label, Safety and Tolerability Study of Triamcinolone Acetonide Administered to the Suprachoroidal Space in Patients with Non-infectious Uveitis

A clinical trial was designed to evaluate the safety and tolerability of a single injection of TA (triamcinolone acetonide administered as TRIESCENCE™) into the SCS in patients diagnosed with non-infectious uveitis.

Each mL of the sterile, aqueous suspension of TRIESENCE™ provides 4 mg of triamcinolone acetonide, with sodium chloride for isotonicity, 0.5% (w/v) carboxymethylcellulose sodium and 0.015% polysorbate 80. It also contains potassium chloride, calcium chloride (dihydrate), magnesium chloride (hexahydrate), sodium acetate (trihydrate), sodium citrate (dihydrate) and water for injection. Sodium hydroxide and hydrochloric acid may be present to adjust pH to a target value 6-7.5.

The primary purpose of this trial was to evaluate the overall safety and tolerability of treating uveitis patients (non-infectious uveitis—intermediate, posterior or pan-uveitis) by administering a triamcinolone into the SCS via a single suprachoroidal injection. Eligibility criteria include adult patients with non-infectious uveitis experiencing either macular edema or vitreous haze, a common complication of uveitis. This was in order to determine whether SCS administration of TA could improve patient vision through reducing the effects of either condition. For inclusion in the trial, patients must have IOP (intra-ocular pressure) of no greater than 22 mmHg

Specifically, the characteristics of the study population were as follows:

Male and non-pregnant females, >18 years old

Non-infectious intermediate, posterior or pan-uveitis

No glaucomatous damage and not a “steroid responder”

BCVA≧20/200 OU, worse eye enrolled

Cystoid macular edema (CME)≧310 _{I}LE or vitreous have ≧1.5+

Further, the following inclusion/exlusion criteria were applied:

Stable systemic immunosuppressive therapy (IMT) for 6 months, stable prednisone for 1 month

No Intravitreal triamcinolone or dexamethasone implant for 6 months

No anti-VEGF intravitreal treatment for 2 months

No difluprednate drops for 1 month

No Retisert® (fluocinolone acetonide intravitreal implant) for 3 years

No ocular surgery within 6 months.

Eight patients (six females, two males) were enrolled and treated. The mean age of the patient population was 56.0 and the age range of patients was from 42 to 78 years. Seven of the patients qualified for the study based on the CME criteria, while four of the patients qualified for the study based on the vitreous haze criteria of ≧1.5.

Each patient enrolled received a single SCS microinjection of 4.0 mg (100 μL) of triamcinolone acetonide of day 1. Patients returned for a follow-up examination on the day after the injection and then for eight additional evaluations at weeks 1, 2, 4, 8, 12, 16, 20 and 26 following the treatment. Patients may receive other treatment at any time during the trial with any accepted therapy based on their physician's best medical judgment, if their condition deteriorates or if the physician otherwise determines it to be advisable. In the event a patient received other treatment, the patient was followed for the duration of the trial for safety purposes, but efficacy measures were no longer considered thereafter.

Patients received the single SCS injection 4 mm posterior to limbus and received an ultrasound assessment of scleral thickness immediately after injection.

Endpoints. The main safety endpoint was changes from baseline in intraocular pressure (TOP). Also assessed was an efficacy endpoint relating to changes in best-corrected visual acuity, or BCVA, as well as changes in excess retinal thickness.

Safety results. All subjects had at least 1 adverse event (AE), and a total of 37 AEs were reported. Most AEs were mild or moderate in severity (95%). Pain was the most commonly reported AE. Specifically, eye pain was reported in 4 subjects. However, all pain AEs were reported as mild and not related to TA SCS injection. One serious event (unrelated pulmonary emboli; SAE) occurred. No deaths have been reported. Approximately half (57%) of the reported AEs were ocular adverse events. Nine ocular AEs in four subjects were considered possibly related to the TA SCS injection.

No significant elevation in IOP was observed in the eight patients, and no patient has required IOP lowering medication.

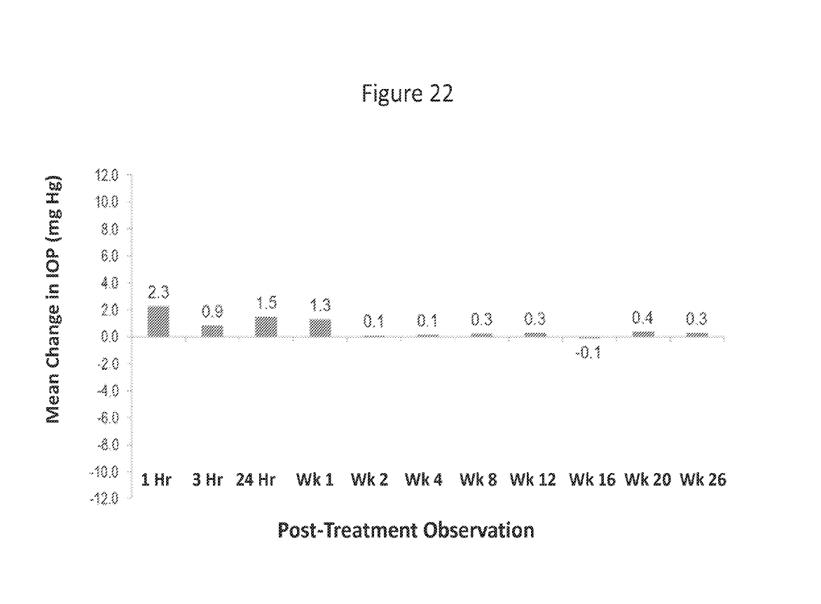

The graph in FIG. 22 shows the mean change in IOP for patients in the trial, as measured at different time points post-treatment. The number of patients included in the results for the various measurement time points below varies because the four patients were treated on different dates and only two patients have currently completed the full 26-week observation period.

In addition to these IOP observations, the drug was considered generally well-tolerated. One patient, who had a history of pulmonary embolisms, was hospitalized for a embolism 10 weeks after the treatment. This serious adverse event was considered to be unrelated to the treatment and resolved after three days.

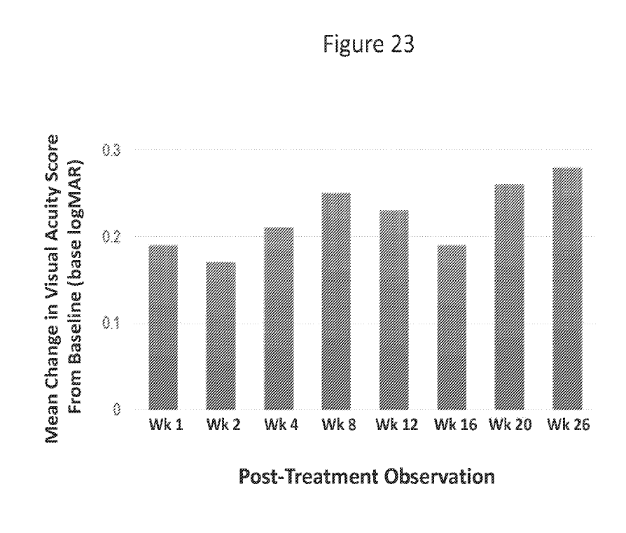

Visual Acuity. BCVA (best corrected visual acuity) was measured for all eight patients. BCVA is a common measurement of a patient's ability to see at distances and changes are measured as the difference in number of letters read on a standard eye chart. FIG. 23 summarizes the mean improvement in BCVA observed. Four of the eight patients showed meaningful improvements in BCVA (a gain of about 3 lines) at 26 weeks following a single suprachoroidal injection of TA on Day 1.

Retinal Thickness. Seven patients were enrolled with macular edema. Change in macular edema was evaluated by measuring change in retinal thickness. A reduction in retinal thickness in patients with macular edema occurs with the removal of excess fluid from the retina, reflecting a reduction of the swelling of the macula and other parts of the retina, affected by the edema.

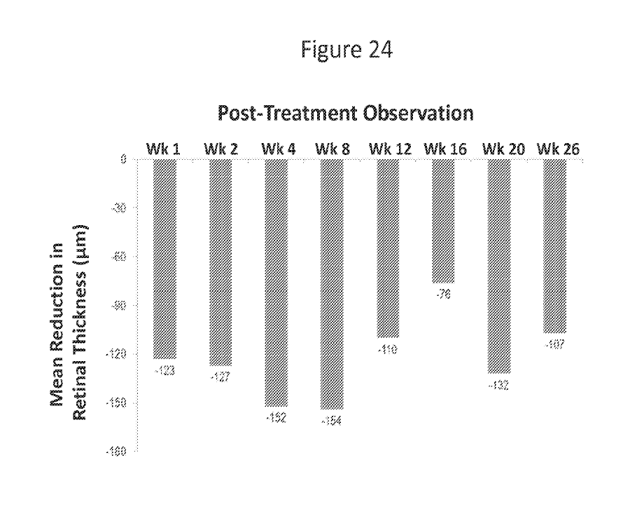

The graph in FIG. 24 summarizes the mean change in retinal thickness observed to date in the trial. The mean reduction in macular edema at week 26 was over 100 microns with a range from 76 to 154 microns reduction over the 26-week post treatment observation period following the single TA injection into the SCS. An average reduction of about 20 percent in CME was observed for the seven patients.

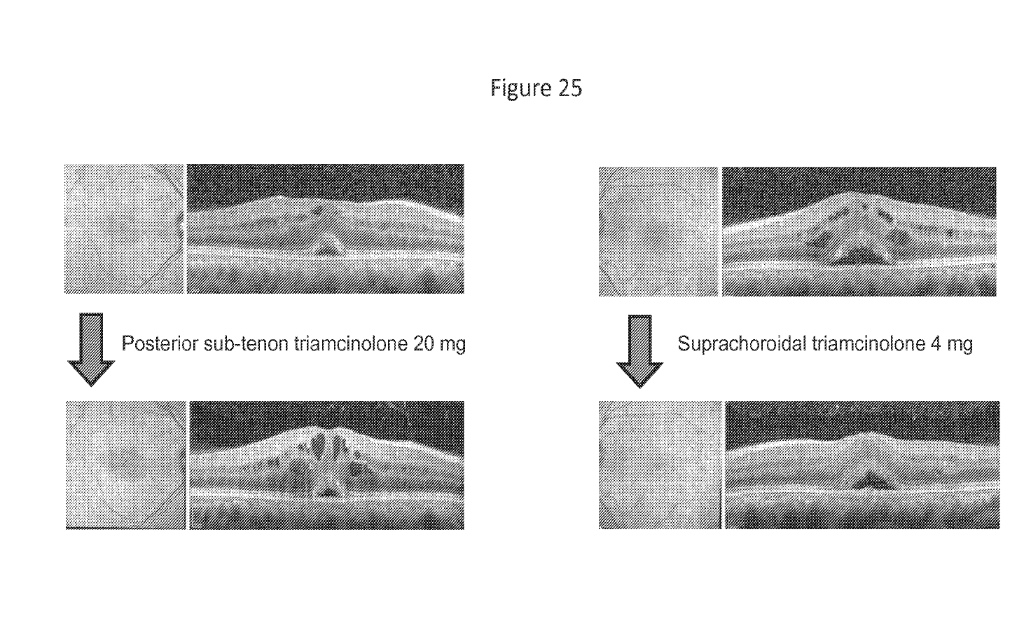

One patient, a 52 year old woman, presented with bilateral uveits with macular edema in both eyes. She was treated with TA via SCS injection in one eye (4 mg TA) and sub-tenon TA injection in the other eye (20 mg TA). FIG. 25 provides OCT images of the eyes of this patient prior to and subsequent to the dosing session. results for this patient (FIG. 25). The eye treated with TA via suprachoroidal injection provides a greater decrease in retinal thickness as compred to the subtenon injection (FIG. 25).

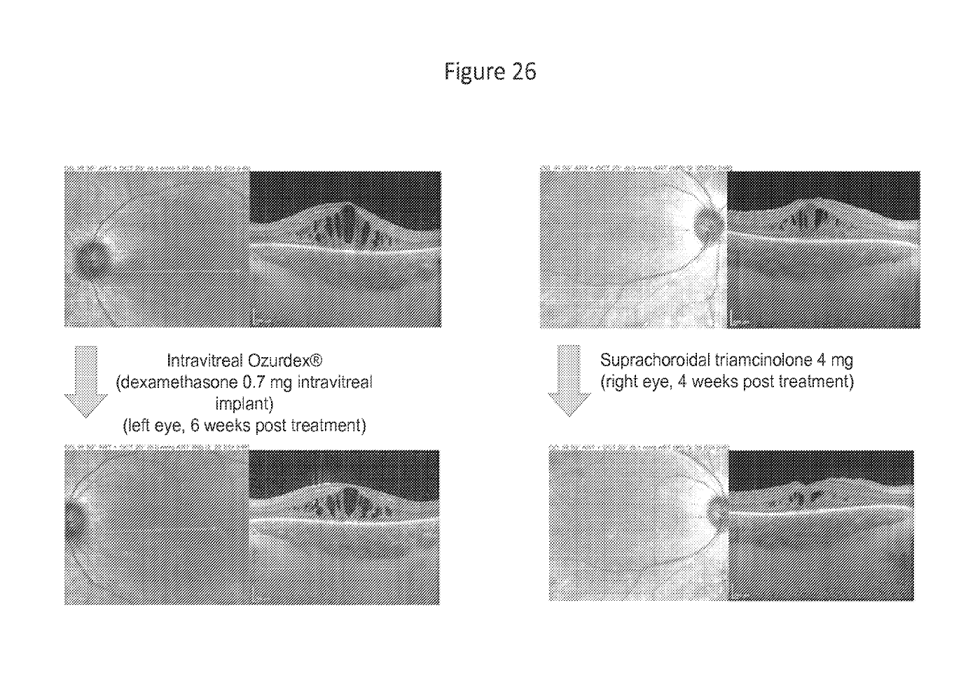

A 25-year old male patient presented with bilateral uveitis, with macular edema in both eyes. The patient was treated with Ozurdex in the left eye and with TA in the right eye. Four to six weeks after treatment, the eye treated with TA via suprachoroidal injection looks better than the intravitreally Ozurdex treated eye (FIG. 26).

### Example 3

### A Randominzed, Masked, Multicenter Study to Assess the Safety and Efficacy of CLS-TA, Triamcinolone Acetonide Injectabile Suspension in the Treatment of Subjects With Macular Edema Following Uveitis

The trial described in this Example is a Phase 2, randomized, masked, multicenter study to assess the safety and efficacy of CLS-TA in the treatment of subjects with ME following non-infectious uveitis. The purpose of this study is to evaluate the safety and efficacy of CLS-TA in subjects with ME following non-infectious uveitis. Two different doses, 4 mg and 0.8 mg, of CLS-TA will each be evaluated for safety and efficacy.

Oral corticosteroids remain the main initial choice in treating patients with uveitis not responding to topical treatment, however their chronic use can be toxic, especially for bones, including osteoporosis or growth retardation. Non-steroidal immunosuppressive agents can either be used to treat uveitis directly, or they are used as corticosteroid sparing therapy or they are used as agents to control refractory uveitis when the condition is sight threatening. Agents commonly used are cyclosporine A, methotrexate, azathioprine, cyclophosphamide and chlorambucil. Cyclosporine is efficacious but is nephrotoxic, particularly in elderly patients. It is rarely sufficient for use as monotherapy, and is infrequently used in uveitis practices. Methotrexate is well tolerated, and has been the usual first line steroid sparing agent for many years. Although it is quite effective in many patients, its onset of action is very delayed (months), and it carries with it the risks of liver toxicity and decreased white blood cell counts (Kalinina 2011). As well, it causes significant fatigue and nausea, making it difficult for some patients to tolerate. It is also absolutely contraindicated in pregnancy (pregnancy category X). Azathioprine is another drug in the same class as methotrexate. It also has a delayed onset of action, and may not be very well tolerated. Mycophenolate mofetil is another steroid sparing agent. It has a somewhat faster onset of action than the other two agents, but can decrease white blood cell count and increase blood pressure. As well, it has significant gastrointestinal side effects. Cyclophosphamide and intravenous steroids are helpful for emergency treatment. Chlorambucil is toxic and carcinogenic, but might lead to an increased rate of remission and may be useful for short term therapy. In short, all of the systemic agents mentioned above carry the risk of significant systemic side effects.

This clinical trial will be conducted in compliance with the protocol, International Conference on Harmonisation (ICH), GCP guidelines, and other applicable regulatory requirements. The study population will include approximately 20 adult subjects, 18 years or older, diagnosed with macular edema (ME) following non-infectious uveitis who meet all of the inclusion criteria, and none of the Exclusion criteria. All subjects will receive a single injection of study drug into a single eye. Approximately 11 U.S. sites will recruit subjects for this study.

The subjects enrolled in this study will be chosen from subjects with ME in the study eye with a retinal thickness of at least 310 microns in the central subfield (average retinal thickness in the central 1 mm) as measured by SD-OCT, using a Heidelberg SPECTRALIS®, and confirmed by the Central Reading Center.

The formulation of triamcinolone used in this study (CLS-TA, triamcinolone acetonide injectable suspension) is an unpreserved, terminally sterilized, aqueous suspension, formulated for administration into the SCS as a single injection of up to 4 mg in 100 microliters (μL), or up to 0.8 mg in 100 microliters (μL) using a microinjector. The drug product is intended for single use. CLS-TA is supplied as a 1.3 mL fill of a 40 mg/mL or 8 mg/mL sterile TA suspension in a 2 mL/13 mm TopLyo® single use vial, with a rubber stopper and an aluminum seal.

The study has 2 arms randomized 4:1. See Table ______ below. Subjects are randomized in a 4:1 ratio to receive a single injection of CLS-TA, 4 mg in a volume of 100 μL or CLS-TA, 0.8 mg in a volume of 100 μL. Study personnel, study patients, the sponsor, and project teams at the Contract Research Organizations (CROs) involved in the study will be masked to treatment assignments. Approximately 20 total subjects at approximately 11 U.S. sites will be enrolled. The study design includes 5 clinic visits over roughly two (2) months. Subjects will be in the study for no more than 70 days. Subjects will receive treatment at Day 1 (Visit 2), approximately 1-10 days after the initial screening visit (Visit 1). They will continue to be monitored for safety and efficacy for 2 months following their injection.

Eligibility will be established at Visit 1 (Screening Visit). Subjects must qualify on SD-OCT readings confirmed by a Central Reading Center prior to being treated. Depending on the treatment arm to which subjects are assigned, subjects will either receive a single suprachoroidal injection of CLS-TA, up to 4 mg in 100 μL, or a single suprachoroidal injection of CLS-TA, up to 0.8 mg in 100 μL in a single eye. Injection is carried out as described in FIG. 21.

If both eyes are eligible (see below for inclusion and exclusion criteria), the eye with worse edema (that with the greater degree of macular thickening per SD-OCT) will be selected. If ME is equivalent in both eyes, the right eye will be chosen.

Subjects remain at the clinic after treatment for at least 30 minutes for evaluation. A follow-up examination will be conducted approximately 7-10 days after the injection procedure (Visit 3). All subjects will return to the clinic monthly for Visits 4-5 (Months 1 and 2). Visit 4 is 28 days ±3 days after treatment at visit 2 and visit 5 is 28 days ±3 days after visit 3. A final evaluation is conducted at Visit 5—End of Study (Month 2). The treatment arms for the study are provided in Table 3, below.

TABLE 3

| Treatment arm | Number of subjects |
|---|---|
| CLS-TA 4.0 mg | ~16 |
| CLS-TA 0.8 mg | ~4 |
| Total subjects | ~20 |

### Endpoints

The primary objective of this study is to determine the safety and efficacy of CLS-TA, with doses up to 4 mg and 0.8 mg, each in a volume of up to 100 μL, by determining change in retinal thickness from baseline in central subfield thickness (CST) as measured by spectral domain optical coherence tomography (SD-OCT) in subjects with ME following non-infectious uveitis. Accordingly, the primary endpoint is the mean absolute change from baseline in CST as measured by SD-OCT after treatment with CLS-TA (4 mg and 0.8 mg) at 2 months in eyes with ME following uveitis.

The safety endpoints of this study are as follows

Incidence of treatment-emergent adverse events (TEAEs) and serious adverse events (SAEs), grouped by organ system, relatedness to study medication, and severity

Percentage of subjects whose IOP increase is above 30 mmHg

Percentage of subjects whose IOP increases >10 mmHg from their own baseline IOP.

The secondary endpoints of the study are to:

Percentage of subjects with a ≧20% reduction in CST after treatment with CLS-TA (4 mg and 0.8 mg) at 1 and 2 months.

Percentage of subjects with a CST of ≦310 um at 1 and 2 months.

Mean change from baseline in BCVA after treatment with CLS-TA (4 mg and 0.8 mg) at 1 and 2 months.

Percentage of subjects who gain ≧5 letters in BCVA at 1 and 2 months compared with baseline [BCVA score based on the Early Treatment of Diabetic Retinopathy Study (ETDRS) visual acuity charts assessed at a starting distance of 4 meters].

Percentage of subjects who gain ≧10 letters in BCVA at 1 and 2 months compared with baseline (BCVA score based on the ETDRS visual acuity charts assessed at a starting distance of 4 meters).

Percentage of subjects who gain ≧15 letters in BCVA at 1 and 2 months compared with baseline (BCVA score based on the ETDRS visual acuity charts assessed at a starting distance of 4 meters)

Percentage of subjects who lose <15 letters in BCVA at 1 and 2 months compared with baseline (BCVA score based on the ETDRS visual acuity charts assessed at a starting distance of 4 meters).

General Inclusion Criteria. Individuals are eligible for participation in this study if they meet the following criteria:

Understand the language of the informed consent and are willing and able to provide written informed consent prior to any study procedures.

Are at least 18 years of age.

Are willing to comply with the instructions and attend all scheduled study visits.

If female, the subject must be non-pregnant, non-lactating and not planning a pregnancy. Females of childbearing potential must agree to use an acceptable method of contraception throughout participation in the study. Acceptable methods of contraception include double barrier methods (condom with spermicide or diaphragm with spermicide), hormonal methods (oral contraceptives, implantable, transdermal, or injectable contraceptives), or an intrauterine contraceptive device (IUCD) with a documented failure rate of less than 1% per year. Abstinence may be considered an acceptable method of contraception at the discretion of the investigator, but the subject must agree to use one of the acceptable birth control methods if she becomes sexually active.

Ophthalmic Inclusion Criteria

Only one eye can be treated under this protocol. If both eyes are eligible, the eye with worse measurement of ME associated with uveitis will be designated as the study eye. Subjects are eligible for participation if their study eye has:

A history of non-infectious uveitis, including anterior, intermediate, posterior or panuveitis.

ME, with or without subretinal fluid, associated with non-infectious uveitis.

A retinal thickness of ≧310 microns in the central subfield (average retinal thickness in the central 1 mm ring, as measured by SD-OCT (using the Heidelberg SPECTRALIS) and confirmed by the Central Reading Center.

ETDRS BCVA score of ≧20 letters read (20/400 Snellen approximate) in each eye.

Exclusion Criteria. An individual is not eligible for participation in this study if they meet any of the following criteria:

Has any uncontrolled systemic disease that, in the opinion of the investigator, would preclude participation in the study (e.g., unstable medical status including uncontrolled elevated blood pressure, cardiovascular disease, and glycemic control) or put the subject at risk due to study treatment or procedures.

Has a likely need for hospitalization or surgery within the study period, including planned elective surgery or hospitalization that cannot be deferred.

Has a known human immunodeficiency virus infection, other immunodeficiency disease or other medical condition for which corticosteroid therapy would be contraindicated in the investigator's opinion.

Has a known hypersensitivity to any component of the formulation of TA, fluorescein, or to topical anesthetics.

Has a systemic infection for which prescription anti-infectious pharmacological therapy is indicated.

Is currently enrolled in an investigational drug or device study or have used an investigational drug or device within 30 days of entry into this study.

Is an employee of the site who is directly involved in the management, administration, or support of this study or is an immediate family member of the same.

History of any serious or active psychiatric illness that, in the opinion of the investigator, would interfere with subject treatment, assessment or compliance with the protocol.

Has used acetazolamide (Diamox®) in the 2 weeks prior to study treatment.

Has taken systemic corticosteroids at doses greater than 20 mg per day for oral prednisone (or equivalent for other corticosteroids) in the 2 weeks prior to study treatment needed to maintain subject care.

Is currently using prescribed nonsteroidal anti-inflammatory drugs (excluding over-the-counter use) or prescribed immunomodulatory therapies, unless the dose has been stable for at least 2 weeks, and no change in dosing is anticipated for the study duration.

Has taken any interferon/fingolimod or any other drug in the 6 weeks prior to study treatment, where the drug is known to induce or exacerbate ME.

Has uncontrolled diabetes.

Ophthalmic Exclusion Criteria. Subjects are ineligible for participation if the subject:

Is monocular.

Has uveitis of infectious etiology.

Has significant media opacity precluding evaluation of the retina and vitreous in the study eye.

Has chronic ME, macular scarring or significant ischemia such that visual acuity is unlikely to improve with treatment per the Investigator's judgment.

Has ME with etiology other than uveitis.

Has an ocular condition that, in the opinion of the Investigator, would put the subject at risk due to study treatment or procedures (i.e. active ocular infection, history of a suprachoroidal hemorrhage, etc.).

Has had uveitis unresponsive to prior systemic corticosteroid treatment in either eye.

Has active ocular disease, other than uveitis in the study eye, or infection including external ocular infections, such as conjunctivitis, herpetic infection, chalazion, or significant blepharitis in either eye.

Has ocular hypertension (IOP>22 mmHg) irrespective of topical treatment or evidence of glaucomatous optic nerve damage in the study eye. Anyone who has a history of clinically significant IOP elevation in response to corticosteroid treatment (“steroid responder”) in the study eye will also be excluded.

Has had a change in IOP lowering medications in the 30 days prior to study treatment.

Has a history of any vitreoretinal surgery (scleral buckle, pars plana vitrectomy, retrieval of a dropped nucleus or intraocular lens, etc.; prior photocoagulation and IVT injections are acceptable) in the study eye. Prior cataract extraction or Yttrium-Aluminum-Garnet (YAG) laser capsulotomy is allowed, but must have been performed at least 3 months prior to treatment.

Has a history of cyclodestructive procedures and multiple filtration surgeries (2 or more) in the study eye.

Has evidence of an epiretinal membrane affecting the macula or vitreomacular traction in the study eye that in the investigator's opinion could prevent improvement in visual acuity.

Has presence of a staphyloma in the study eye.

Demonstrates the presence of a toxoplasmosis scar in the study eye.

Has eye diseases other than uveitis that could compromise central visual acuity (e.g. clinically significant diabetic retinopathy, scleritis, ischemic optic neuropathy or retinitis pigmentosa) in the study eye.

Has high myopia defined as a spherical equivalent >−6 diopters or an axial length ≧26 mm in the study eye.

Has any condition in the study eye that in the opinion of the Investigator, may predispose to scleral thinning.

Has any ocular trauma, within the immediate 6 months prior to study treatment, in the study eye.

Has had photocoagulation or cryotherapy within the 6 months prior to study treatment in the study eye.

Has had any IVT injection of anti-VEGF treatment (bevacizumab, aflibercept, pegaptanib or ranibizumab) in the 2 months prior to study treatment in the study eye.

Has had any ophthalmic topical corticosteroid within 10 days of study treatment, injection of periocular or intraocular corticosteroids within 60 days of study treatment, Ozurdex® implant in the 120 days prior to the study treatment, or any prior use of Retisert™ or Iluvien™ implant in the study eye in the past 1 year prior to study treatment.

Has had a previous suprachoroidal injection of TA in the study eye in the past 30 days.

Randomization Criteria. Subjects are eligible for randomization at Visit 2 if the following criteria are met:

Central Reading Center confirmation of ME by SD-OCT (from Visit 1 OCT data), with or without subretinal fluid, caused by non-infectious uveitis (per the Investigator's judgment) in the study eye.

Central Reading Center confirmation of a retinal thickness of ≧310 microns in the central subfield (average retinal thickness in the central 1 mm ring, as measured by SD-OCT using the Heidelberg SPECTRALIS®) from the Visit 1 OCT data.

Subject continues to meet inclusion/exclusion criteria.

Post-Injection Procedures. The following assessments must occur following the injection (Visit 2):

Assess for AEs

Review changes to concomitant medications
Measure seated, resting heart rate and blood pressure

Perform ophthalmic assessments on the study eye only
Perform slit-lamp biomicroscopy
Evaluate IOP 30 (±5) minutes post injection • If IOP remains elevated, subject must remain on site until IOP is under control per investigator's best medical judgment.
Perform indirect ophthalmoscopy

Schedule subject to return for Visit 3.

Visit 3 occurs 7 to 10 days post Visit 2 (Randomization/Treatment). During Visit 3, the following procedures will be performed:

Assess for AEs

Review changes to concomitant medications

Measure seated, resting heart rate and blood pressure

Perform ophthalmic assessments on the study eye only
BCVA exams performed by certified site personnel using the ETDRS protocol
Perform slit-lamp biomicroscopy
Evaluate IOP
Perform dilated indirect ophthalmoscopy
Obtain FP-4W field color fundus photographs and upload to the Central Reading Center
Obtain SD-OCT images and upload to the Central Reading Center

Schedule subject to return for Visit 4

Visit 4 occurs approximately 1 month post injection. The visit should be 28±3 days from Visit 2. During Visit 4, the following procedures will be performed:

Assess for AEs

Review changes to concomitant medications

Measure seated, resting heart rate and blood pressure

Perform ophthalmic assessments on the study eye only
BCVA exams performed by certified site personnel using the ETDRS protocol
Perform slit-lamp biomicroscopy
Evaluate IOP
Perform indirect ophthalmoscopy
Obtain SD-OCT images and upload to the Central Reading Center

Schedule subject to return for next visit

Visit 5 will be the final evaluation visit and exit from the study. Visit 5 occurs within 56 ±4 days from Visit 2. The following procedures will be performed:

Assess for AEs

Review changes to concomitant medications

Measure seated, resting heart rate and blood pressure

Perform urine pregnancy test on females of childbearing potential

Perform ophthalmic assessments on both eyes (except FA; study eye only)
BCVA exams performed by certified site personnel using the ETDRS protocol
Perform slit-lamp biomicroscopy
Evaluate IOP
Perform dilated indirect ophthalmoscopy
Obtain FP-4W field color fundus photographs and upload to the Central Reading Center
Obtain SD-OCT images and upload to the Central Reading Center
Perform FA with the early series of the study eye and upload to the Central Reading Center.

### Efficacy Assessments

Central Subfield Thickness as measured by SD-OCT will be assessed as a measure of efficacy. Each site will be provided an imaging protocol and submission procedures from the Central Reading Center. The SD-OCT instrument and technician must be certified prior to submission of study data. They will be trained on imaging and uploading images to EyeKor's Excelsior system for this specific protocol. Retinal thickness and disease characterization will be assessed via SD-OCT (Heidelberg SPECTRALIS®) at every visit. OCT will be performed on both eyes at Visits 1 and 5, and in the study eye only at Visits 2, 3 and 4.

The Central Reading Center will evaluate study images in a masked, independent manner. At Screening (Visit 1), the Central Reading Center will confirm subject eligibility on the basis of retinal thickness criteria prior to subject enrollment. Upon confirmation from the Central Reading Center via email, the site may proceed with qualifying the subject for Randomization/Treatment which will occur at Visit 2.

SD-OCT submissions will include a volume (cube) scan consisting of 49 B-scans of 6 mm length centered on the fovea. An additional Enhanced Depth Imaging (EDI) scan will be obtained horizontally through the fovea. SD-OCT scans will be evaluated for quality and any segmentation errors affecting the measurement of central subfield retina thickness will be corrected. Additional evaluation outputs will include macular grid volume and assessment of retinal and choroidal anatomy.

BCVA assessed using the ETDRS protocol will also be assessed. Each site will have at least one certified exam lane that includes all required equipment to assess BCVA by one or more certified visual acuity examiner. Training/certification on the ETDRS protocol will be completed prior to subject enrollment. In addition, ETDRS training/certification documentation will be kept on site and with the sponsor. Site staff will be masked to treatment. BCVA will be assessed at every visit. BCVA will be measured on both eyes at Visits 1 and 5, and in the study eye only at Visits 2, 3 and 4.

Safety and tolerability will be evaluated using the following assessments:

Intraocular pressure. Tonopen or Goldmann Applanation Tonometer is allowed for measuring IOP, however, an average of 3 measurements should be used. Mean IOP values should be rounded up to the next whole number if the value is greater than or equal to 0.5 mmHg and rounded down if less than 0.5 mmHg All instruments used to measure IOP must be calibrated according to the manufacturer's specifications and documented (i.e., calibration log). The same tool for measuring IOP should be used for every visit. IOP will be measured at every visit. It will be measured on both eyes at Visits 1 and 5 and the study eye only at Visits 2, 3 and 4.

Slit-lamp biomicroscopy. Slit-lamp biomicroscopy will be performed using the investigator's standard slit lamp equipment and procedure. This procedure should be the same for all subjects observed at the investigator's site. Observations for each eye should be made for the following variables (including but not limited to): conjunctiva, cornea, lens, anterior chamber, iris, and pupil. Slit-lamp biomicroscopy will be assessed at every visit. It will be measured on both eyes at Visits 1 and 5 and the study eye only at Visits 2, 3 and 4.

Indirect ophthalmoscopy. Dilated ophthalmoscopy should be performed according to the investigator's standard dilation procedure. This procedure should be the same for all subjects observed at the investigator's site. The fundus will be examined thoroughly and the following variables (including but not limited to): vitreous haze, vitreous, retina, choroid, and optic nerve/disc. Dilated indirect ophthalmoscopy will be assessed at every visit. It will be measured on both eyes at Visits 1 and 5 and the study eye only at Visits 2, 3 and 4.

Fluorescein Angiogram. It is recommended that when both fundus photos and FA are conducted in the same visit that the Fundus Photos be taken first. Digital equipment will be registered and photographers certified for the imaging procedures. The same equipment should be used throughout the study. All testing should be carried out by the same operator, whenever possible, on all subjects per research site. The designated person must be on the site delegation log. It is recommended that a backup also be named. All data/images will be uploaded to EyeKor's Excelsior system. As a reminder all images should be de-identified before uploading. FA will be performed at Visits 1 and 5 on the study eye only. Anatomic assessments will include the area of fluorescein leakage, area of capillary nonperfusion, the presence of retinal vascular and optic nerve head staining, and retinal pigment epithelium abnormalities.

Fundus Photographs. FP-4W fields (4 standard Wide Angle Fields). The same camera should be used throughout the study. All photos should be taken by the same photographer, whenever possible, on all subjects per research site. De-identified images will be uploaded to EyeKor's Excelsior system. Fundus photographs will be taken at Visits 1 and 5 on both eyes and the study eye only at Visit 3. Characteristics graded from fundus photographs includes vitreous haze score, lesions consistent with posterior uveitis, optic disc swelling, and vascular abnormalities.

Vitreous Haze. Photographic vitreous haze will be assessed clinically at every visit via indirect ophthalmoscopy using a standardized photographic scale ranging from 0 to 4, with 0-4 defined below in Table 4 (Nussenblatt 1985 as modified in Lowder 2011). Vitreous haze will also be graded from the color fundus photographs according to a similar scale. It will be assessed on both eyes at Visits 1 and 5, and in the study eye only at Visits 2, 3 and 4.

TABLE 4
Vitreous Haze Scale

| Score | Description |
|---|---|
| 0 | No inflammation |
| +0.5 | Trace inflammation (slight blurring of the optic disc margins |
| | and/or loss of the nerve fiber layer reflex) |
| +1 | Mild blurring of the retinal vessels and optic nerve |
| +1.5 | Optic nerve head and posterior retina view obsuration greater |
| | than +1 but less than +2 |
| +2 | Moderate blurring of the optic nerve head |
| +3 | Marked blurring of the optic nerve head |
| +4 | Optic nerve head not visible |

### Example 4

### Safety and Efficacy of Suprachoroidal CLS-TA in Combination with Intravitreal Aflibercept in Subjects with Macular Edema Following Retinal Vein Occlusion

This Phase 2, multicenter, randomized, active-controlled, masked, parallel arm study seeks to evaluate the safety and efficacy of a single suprachoroidal injection of CLS-TA given concomitantly with an intravitreal (IVT) injection of aflibercept compared to IVT aflibercept alone in subjects with macular edema (ME) following retinal vein occlusion (RVO). RVO is a condition that affects vision, resulting from a blockage in one of the veins returning blood flow from the retina. RVO is the second most common cause of vision loss due to retinal vascular disease.

This study assesses the safety and efficacy of a suprachoroidal injection of CLS-TA plus IVT aflibercept compared to subjects administered a sham suprachoroidal procedure plus IVT aflibercept in the treatment of subjects with ME following retinal vein occlusion (RVO). Each subject will receive at least one IVT aflibercept injection and approximately half of the subjects will receive a single suprachoroidal injection of CLS-TA. The subjects enrolled in this study will be treatment naïve RVO subjects (HRVO, CRVO and BRVO) with ME in the study eye. All qualifying subjects will be randomized (Day 1) to receive an IVT injection of an anti-VEGF treatment (aflibercept) plus a suprachoroidal injection of CLS-TA or an IVT injection of aflibercept plus a sham suprachoroidal procedure. Subjects will be followed for approximately 3 months following randomization. The subject, sponsor, visual acuity technician and the optical coherence tomography (OCT) reading center will be masked to treatment.

Approximately 40 subjects at approximately 10 U.S. sites will be enrolled. The study design includes 5 clinic visits and one safety phone call over approximately three (3) months. Subject eligibility will be established at Visit 1 during the screening process (Day −14 to −1) where subjects must qualify on spectral-domain optical coherence tomography (SD-OCT) readings confirmed by a Central Reading Center (CRC) prior to being treated. Eligible subjects will return to the clinic for Visit 2—Randomization (Day 1) where subjects will be randomized via the interactive web response system (IWRS). Subjects will be randomized to receive either an IVT aflibercept injection followed by a suprachoroidal CLS-TA injection or an IVT aflibercept injection followed by a suprachoroidal sham procedure. Subjects remain at the clinic after the suprachoroidal CLS-TA injection or sham for about 30 minutes for evaluation. A follow-up safety phone call will be required on Day 2 (24-48 hours following treatment). Subjects will then receive an IVT aflibercept injection at Visits 3 (Month 1) and 4 (Month 2) only if criteria for additional therapy are met. If subjects do not qualify for the IVT injection of aflibercept, they will be given a sham IVT aflibercept procedure. Subjects will have their final evaluation conducted at Visit 5—End of Study (Month 3). No study injections will occur at Visit 5.

### Endpoints

The primary endpoint is the total number of times subjects qualify to be administered IVT aflibercept in each arm through Month 3.

Safety Endpoints

Incidence of TEAEs and SAEs, grouped by organ system, relatedness to study medication, and severity.

Incidence of changes in safety parameters as described in Section 8.1, including: IOP, slit lamp biomicroscopy, indirect ophthalmoscopy, imaging parameters and vital signs.

Secondary Endpoints

Total number of aflibercept treatments in each arm at Month 1, at Month 2 and at Month 3.

Percentage of subjects with a CST of ≦310 nm at 1, 2 and 3 months.

Mean change from baseline in CST at 1, 2 and 3 months.

Mean change from baseline in BCVA at 1, 2 and 3 months.

Percentage of subjects who gain ≧15 letters in BCVA at 1, 2 and 3 months compared with baseline.

Percentage of subjects who lose <15 letters in BCVA at 1, 2 and 3 months compared with baseline.

Trial Treatments

CLS-TA, triamcinolone acetonide injectable suspension, is a sterile aqueous suspension formulated for administration into the eye. The drug product is terminally sterilized and is intended for single use. CLS-TA is supplied as a 1.3 mL fill of 40 mg/mL sterile CLS-TA suspension in a 2 mL/13 mm TopLyo® single use vial, with a rubber stopper and an aluminum seal. CLS-TA must be stored under ambient temperature conditions at ca. 20°-25° C. (68°-77° F.); do not freeze. Protect from light by storing in the kit.

The 4 mg dose of CLS-TA contains 40 mg/mL of TA. Subjects will be randomized 1:1 to receive a single suprachoroidal injection of 40 mg/mL (4 mg in 100 μL) CLS-TA (Active arm) or a sham suprachoroidal procedure (Control arm). This will be based on the randomization code and what is assigned via the IWRS.

EYLEA® (aflibercept) Injection is an FDA approved prescription medicine for the treatment of RVO. In this study, the dosage for EYLEA® is 2 mg (0.05 mL) administered by intravitreal injection. Aflibercept will be acquired commercially by the clinical sites.

All qualifying subjects will be randomized on Day 1 to one of the following arms and will receive:

ACTIVE ARM: an IVT injection of aflibercept [2 mg (0.05 mL)] plus a suprachoroidal injection of CLS-TA [4 mg (100 μL)] or

CONTROL ARM: an IVT injection of aflibercept [2 mg (0.05 mL)] plus a sham suprachoroidal procedure.

Subjects randomized to the Active arm (those receiving CLS-TA) or the Control arm will be retreated with an IVT injection of aflibercept at Visits 3 (Month 1) and 4 (Month 2) only if criteria for additional therapy are met. If they do not qualify for the IVT injection of aflibercept, they will be given a sham IVT aflibercept procedure. The Clearside microinjector is designed for suprachoroidal administration of drug through the SCS. The microinjector used for injection into the SCS will be supplied to the site.

Retreatment Criteria. If any of the following criteria is met in the study eye at Month 1 and 2 (Visits 3 and 4), retreatment with an IVT injection of aflibercept is required. Dosing is to be done per the current package insert.

Macular edema or subretinal fluid (new or persistent) in conjunction with a CST ≧340 microns as measured by SD-OCT.

A decrease in BCVA of 10 letters (ETDRS) or greater between the current visit and the BCVA reading from the previous visit.

A decrease in BCVA of 10 letters (ETDRS) or greater from the best measurement (during the study) with an increase in CST of >50 microns from the previous visit, associated with new fluid.

At month 1 and 2 (Visits 3 and 4), if a subject does not qualify for retreatment with an IVT injection of aflibercept, an IVT aflibercept sham procedure will be performed.

### Enrollment Criteria

General Inclusion Criteria. An individual is eligible for participation in this study if he/she meets the following criteria: (1) Understand the language of the informed consent and is willing and able to provide written informed consent prior to any study procedures; (2) Is at least 18 years of age; (3) Is willing to comply with the instructions and attend all scheduled study visits; (4) if female, the subject must be non-pregnant, non-lactating and not planning a pregnancy. Females of childbearing potential must agree to use an acceptable method of contraception throughout participation in the study.

Ophthalmic Inclusion Criteria. Individuals are eligible for participation in this study if he/she meet the following criteria: (1) Clinical diagnosis of ME following RVO in the study eye; (2) CST of ≧310 microns (average retinal thickness in the central 1mm ring) in the study eye as measured by SD-OCT (using the Heidelberg SPECTRALIS®) with or without subretinal fluid and confirmed by the CRC; (3) ETDRS BCVA score of ≧20 letters read (20/400 Snellen equivalent) in each eye, and ≦70 letters read (20/40 Snellen equivalent) in the study eye; (4) Macular Edema with the following characteristics: a. Involving the fovea, b. Due to any RVO and not due to other causes of ME, c. History of ME ≦12 months, d. Visual acuity decrease due to edema.

General Exclusion Criteria. Individuals are ineligible for participation in this study if he/she meet the following criteria: (1) Has any uncontrolled systemic disease that, in the opinion of the investigator, would preclude participation in the study (e.g., infection uncontrolled elevated blood pressure, cardiovascular disease, and glycemic control) or put the subject at risk due to study treatment or procedures; (2) Myocardial infarction or stroke within 90 days of treatment; (3) Any new or change in an existing prescription medication within 30 days of randomization; (4) Has taken systemic corticosteroids at doses greater than 10 mg per day for oral prednisone (or equivalent for other corticosteroids) in the 30 days prior to study treatment needed to maintain subject care for stable, non-exclusionary medical conditions; (5) Has a likely need for hospitalization or surgery within the study period, including planned elective surgery or hospitalization; (6) Has a known human immunodeficiency virus infection, other immunodeficiency disease or other medical condition for which corticosteroid therapy would be contraindicated according to best medical judgment; (7) Has a known hypersensitivity to any component of the formulation of TA, aflibercept, fluorescein, or to topical anesthetics; (8) Is currently enrolled in an investigational drug or device study or have used an investigational drug within 30 days of entry into this study or participated in an ocular device study in the last 90 days; (9) Is an employee of the site who is directly involved in the management, administration, or support of this study or is an immediate family member of the same.

Ophthalmic Exclusion Criteria. An individual is ineligible for participation in this study if he/she meets the following criteria: (1) Has had any IVT injection of anti-VEGF (bevacizumab, aflibercept, pegaptanib or ranibizumab) for RVO in the study eye; (2) In the study eye, any intraocular and periocular corticosteroid injection in the 3 months prior to treatment, OZURDEX® implant in the 6 months prior to treatment, RETISERT™ implant in the 1 year prior to treatment or ILUVEIN® implant in the 3 years prior to treatment; (3) Evidence of or history of any ophthalmic condition in the study eye, other than RVO, that, in the investigator's opinion, might compromise visual acuity (e.g., AMD, diabetic retinopathy, retinal detachment, central serious chorioretinopathy, scleritis, optic neuropathy or retinitis pigmentosa); (4) History of any vitreoretinal surgery (scleral buckle placement, pars plana vitrectomy, retrieval of an intraocular lens, sheathotomy) ever, in the study eye or any ocular surgery in the 3 months prior to randomization. History of IVT injections are allowed; (5) History of an ocular procedure or condition, within the 3 months prior to randomization, or condition that, in the investigator's opinion, could compromise globe or retinal integrity (e.g., staphyloma, cryotherapy, high myopia [defined as a spherical equivalent >−8 diopters], predisposition to scleral thinning, etc.) in the study eye; (6) An ocular condition that in the opinion of the Investigator would put the subject at risk due to study treatment or procedures in the study eye (e.g., active ocular infection, history of a suprachoroidal hemorrhage, chalazion, significant blepharitis); (7) In the study eye, >3 treatments of macular laser photocoagulation. Previous macular laser photocoagulation must have been >60 days prior to injection. Panretinal photocoagulation is allowed; (8) Significant media opacity precluding evaluation of retina and vitreous in the study eye. This includes significant hemorrhage or cataract that is felt to be a major contributor to reduced visual acuity; (9) A study eye that, in the investigator's opinion, would not benefit from resolution of ME, such as eyes with foveal atrophy, dense pigmentary changes, chronic ME greater than 12 months or dense subfoveal hard exudates; (10) Uncontrolled ocular hypertension (IOP >22 mmHg) irrespective of topical treatment or evidence of glaucomatous optic nerve damage in the study eye; (11) Have a history of glaucoma surgery (filtration surgery/trabeculectomy or tube shunt) in the study eye; (12) Have a history of clinically significant IOP elevation in response to corticosteroid treatment (“steroid responder”); (13) Have used any systemic or topical ophthalmic nonsteroidal anti-inflammatory drugs (NSAIDs) to treat ophthalmic conditions in the 1 month prior to treatment; (14) Have had a previous suprachoroidal injection of TA in the study eye.

Randomization Criteria. Subjects are eligible for randomization at Visit 2 if the following criteria are met: (1) CRC confirmation of ME by SD-OCT (from Visit 1 OCT data), with or without subretinal fluid, caused by RVO in the study eye; (2) CRC confirmation of a retinal thickness of ≧310 microns in the central subfield from the Visit 1 SD-OCT data; (3) The study eye gained no more than 10 letters of vision between the Screening visit and Randomization (Visit 2) in the study eye; (4) Subject continues to meet all of the inclusion and none of the exclusion criteria.

General Procedures. The study will consist of 5 study visits and one safety phone call over a maximum of 101 days (14 weeks). Subjects will attend all study visits. All ocular assessments at Visit 1 and Visit 5 will be performed on both eyes except for fluorescein angiography (FA) which will be performed on the study eye only. Ocular assessments at all other visits (Visits 2-4) will be performed on the study eye only. Subjects will be screened for entry (Visit 1) and then return to the clinic within 14 days to be randomized/treated (Visit 2). At randomization, subjects will receive a single IVT injection of aflibercept (per package insert) into the study eye, followed by a single, unilateral, suprachoroidal injection of CLS-TA or a suprachoroidal sham procedure in the study eye, depending on the randomization code assigned. Subjects will remain in the clinic for approximately 30 minutes following the suprachoroidal injection or sham and be assessed for safety. Subjects will receive a Safety Phone Call from the site 24-48 hours post injection and then return 1 month post injection for evaluation (Visit 3). Additional follow-up visits will occur at Months 2 and 3 (Visits 4 and 5).

Visit 1-Screening (Day −14 to −1). At Visit 1, subjects will be screened for eligibility. Before any study-specific assessments are performed, written informed consent will be obtained for each subject. During Visit 1, the following assessments will be performed:

Obtain written informed consent

Assign subject number

Collect demographics, medical and ocular history

Review current and past concomitant medications

Measure seated, resting heart rate and blood pressure

Collect blood and urine for central lab tests prior to FA

Perform ophthalmic assessments on both eyes (except FA; study eye only)
BCVA exams performed by certified site personnel using the ETDRS protocol
Perform slit-lamp biomicroscopy
Evaluate IOP
Perform dilated indirect ophthalmoscopy
Obtain SD-OCT images and upload to the CRC
Obtain 4 Wide Field Color Fundus Photographs (FP-4W) and upload to the CRC
Perform FA with the early series of the study eye and upload to the CRC
Verify subject eligibility based on Inclusion/Exclusion requirements
Determine study eye based upon eligibility criteria. If both eyes are eligible, the eye with worse edema (that with the greater degree of macular thickening per SD-OCT) will be selected. If ME is equivalent in both eyes, the right eye will be chosen.
Perform brief physical exam
Schedule subject to return for Visit 2, Randomization/Treatment.

Visit 2-Randomization/Treatment (Day 1). Visit 2 must occur within 14 days of Visit 1 (Screening) and may only occur once subject is eligible for treatment which includes central lab results being received and reviewed, and confirmation of eligibility by the CRC. No subject may be treated without CRC confirmation of qualifying disease and CST ≧310 microns. Once qualification is confirmed, subjects will be randomized via the IWRS. All qualifying subjects will be randomized (Day 1) to receive either:

ACTIVE: an IVT injection of aflibercept [2 mg (0.05 mL)] plus a suprachoroidal injection of CLS-TA [4 mg (100 μL)] or CONTROL: an IVT injection of aflibercept [2 mg (0.05 mL)] plus a sham suprachoroidal procedure.

Subjects randomized to the Active arm (those receiving CLS-TA) or the Control arm will be retreated with an IVT injection of aflibercept at Visits 3 (Month 1) and 4 (Month 2) only if criteria for additional therapy are met. If they do not qualify for the IVT injection of aflibercept, they will be given a sham IVT aflibercept injection.

Pre-injection procedures. The following must be performed immediately prior to the IVT aflibercept injection:

Assess for AEs

Review changes to concomitant medications

Review central lab results for any significant abnormalities that would exclude the subject from entry

Review the results received from the CRC to confirm that subject is eligible based on disease and CST

Review eligibility based on Inclusion/Exclusion and Randomization criteria

Measure seated, resting heart rate and blood pressure

Perform urine pregnancy test on females of childbearing potential

Perform ophthalmic assessments on the study eye only.
BCVA exams performed by certified site personnel using the ETDRS protocol (remember BCVA technician is to be masked to treatment assignment)
Perform slit-lamp biomicroscopy
Evaluate IOP
Perform indirect ophthalmoscopy
Obtain SD-OCT images and upload to the Central Reading Center (Visit 1 images will be used for qualification; Visit 2 pre-dose images will be used as baseline)

Log onto the IWRS system and randomize subject. Kit number will be assigned.

IVT Injection of aflibercept: Prepare study eye for IVT injection of aflibercept. Administer aflibercept IVT injection per package insert. It is recommended that the intravitreal injection and the suprachoroidal injection are approximately 2 clock hours apart. The superior temporal quadrant is the recommended location for suprachoroidal injections.

Suprachoroidal Injection of CLS-TA (ACTIVE KIT): Suprachoroidal injection should be administered following the IVT injection of aflibercept when the study eye IOP is <30 mmHg, either spontaneously or by treatment, as determined by the Investigator. The injection of 100 μL of CLS-TA is administered into the SCS of the study eye using the Clearside microinjector approximately 2 clock hours from where the IVT aflibercept was administered, preferably in the superior temporal quadrant. See FIG. 21 for method.

Suprachoroidal sham procedure (CONTROL KIT): Sham procedure is administered following the IVT injection of aflibercept when the study eye IOP is <30 mmHg, either spontaneously or by treatment, as determined by the Investigator. The eye is prepared as it would for a suprachoroidal CLS-TA injection. A mock suprachoroidal injection to the study eye is performed.

Post-Injection Procedures. The subjects remain on site for observation for approximately 30 minutes after injection. The following assessments occur following the IVT injection and suprachoroidal injection or sham procedure: (1) Assess retinal artery for perfusion; (2) Assess for AEs; (3) Review changes to concomitant medications; (4) Measure seated, resting heart rate and blood pressure; (5) Perform ophthalmic assessments on the study eye only (a. slit-lamp biomicroscopy; b. Evaluate IOP 10-30 minutes post injection; c. Perform indirect ophthalmoscopy). If IOP remains elevated, subject must remain on site until IOP is under control per investigator's best medical judgment. If IOP is <30 mmHg, the subject may leave the clinic.

Visits 3 (Month 1 Post Injection Follow-Up (Day 28±3)) Visit 4 (2 Month Post Injection Follow-Up (Day 56±3)). Visit 3 occurs approximately 1 month post Visit 2 (Randomization/Treatment). The visit is 28±3 days from Visit 2. Visit 4 occurs approximately 2 months post Visit 2 (Randomization/Treatment). Visit 4 is 56±3 days from Visit 2. During Visits 3 and 4, the following procedures are performed:

Assess for AEs

Review changes to concomitant medications

Measure seated, resting heart rate and blood pressure

Perform ophthalmic assessments on the study eye only
BCVA exams performed by certified site personnel using the ETDRS protocol
Perform slit-lamp biomicroscopy
Evaluate IOP
Perform dilated indirect ophthalmoscopy
Obtain SD-OCT images and upload to the CRC
OPTIONAL: Perform FA with the early series of the study eye and upload to the CRC only if the investigator feels it is necessary for medical judgment.

Administer IVT aflibercept to subjects only if they qualify for additional treatment. If subject does not qualify for additional treatment, administer an IVT sham procedure.

Visit 5-Month 3; End of Study Visit (Day 84±4 days). Visit 5 is the final evaluation visit and exit from the study. Visit 5 occurrs within 84±4 days from Visit 2. During Visit 5, the following procedures are performed:

Assess for AEs

Review changes to concomitant medications

Measure seated, resting heart rate and blood pressure

Perform ophthalmic assessments on the study eye only
BCVA exams performed by certified site personnel using the ETDRS protocol
Perform slit-lamp biomicroscopy
Evaluate IOP
Perform dilated indirect ophthalmoscopy
Obtain SD-OCT images and upload to the CRC
OPTIONAL: Perform FA with the early series of the study eye and upload to the CRC only if the investigator feels it is necessary for medical judgment.

Assessments of efficacy is as follows.

Central Subfield Thickness as measured by SD-OCT. Retinal thickness and disease characterization will be assessed via SD-OCT (Heidelberg SPECTRALIS®) at every visit. OCT will be performed on both eyes at Visits 1 and 5, and in the study eye only at Visits 2, 3 and 4. The CRC will evaluate study images in a masked, independent manner. At Screening (Visit 1), the CRC will confirm subject eligibility on the basis of retinal thickness criteria prior to subject enrollment. Upon confirmation from the CRC via email, the site may proceed with qualifying the subject for Randomization/Treatment which will occur at Visit 2. SD-OCT submissions will include a volume (cube) scan consisting of 49 B-scans of 6 mm length centered on the fovea. An additional Enhanced Depth Imaging scan will be obtained horizontally through the fovea. SD-OCT scans will be evaluated for quality and any segmentation errors affecting the measurement of central subfield retina thickness will be corrected. Additional evaluation outputs will include macular grid volume and assessment of retinal and choroidal anatomy.

BCVA assessed using the ETDRS protocol. BCVA is assessed at every visit. BCVA will be measured on both eyes at Visits 1 and 5, and in the study eye only at Visits 2, 3 and 4.

Safety and tolerability will be evaluated using the following assessments.

Intraocular pressure. Tonopen or Goldmann Applanation Tonometer is allowed for measuring IOP. Mean IOP values are rounded up to the next whole number if the value is greater than or equal to 0.5 mmHg and rounded down if less than 0.5 mmHg All instruments used to measure IOP are calibrated according to the manufacturer's specifications and documented (i.e., calibration log). The same tool for measuring IOP should be used for every visit. IOP is measured at every visit. It will be measured on both eyes at Visits 1 and 5 and the study eye only at Visits 2, 3 and 4.

Slit-lamp biomicroscopy. Slit-lamp biomicroscopy is performed using the investigator's standard slit lamp equipment and procedure. Observations for each eye are made for the following variables (including but not limited to): conjunctiva, cornea, lens, anterior chamber, iris, and pupil. Slit-lamp biomicroscopy is assessed at every visit. It will be measured on both eyes at Visits 1 and 5 and the study eye only at Visits 2, 3 and 4.

Indirect ophthalmoscopy. The fundus is examined thoroughly and the following variables (including but not limited to): vitreous, retina, choroid, and optic nerve/disc. Dilated indirect ophthalmoscopy will be assessed at every visit. It will be measured on both eyes at Visits 1 and 5 and the study eye only at Visits 2, 3 and 4.

Fluorescein Angiogram (FA). All data/images are uploaded to EyeKor's Excelsior system. As a reminder all images should be de-identified before uploading. FA will be performed at Visits 1 and 5 on the study eye only. FA may be performed (it is optional) at Visits 3 and 4 if the investigator feels it is necessary for medical judgment only. Anatomic assessments include the area of fluorescein leakage, area of capillary nonperfusion, the presence of retinal vascular and optic nerve head staining, and retinal pigment epithelium abnormalities.

Fundus Photographs. FP-4W (4 Wide Field Color Fundus Photography). Fundus photographs are taken at Visits 1 and 5 on both eyes. Characteristics graded from fundus photographs includes optic disc swelling, and vascular abnormalities.

### Example 5

### Study Comparing the Effects of SCS and Intravitreal Injections of Triesence in Rabbits

A study was conducted in rabbits to compare the results of SCS injections against results of intravitreal injections with commercially available TA, Triesence, related to the distribution of Triesence through the different tissues of the eye as well as measuring the drug levels in the plasma using our microinjector.

In this study, each rabbit received a single dose of 4.0 mg of Triesence on day 1 of the study injected either intravitreally or into the SCS. The rabbits were then observed for periods of up to 90 days and the concentration of Triesence in various parts of the eye was measured at days 14, 28, 56 and 91.

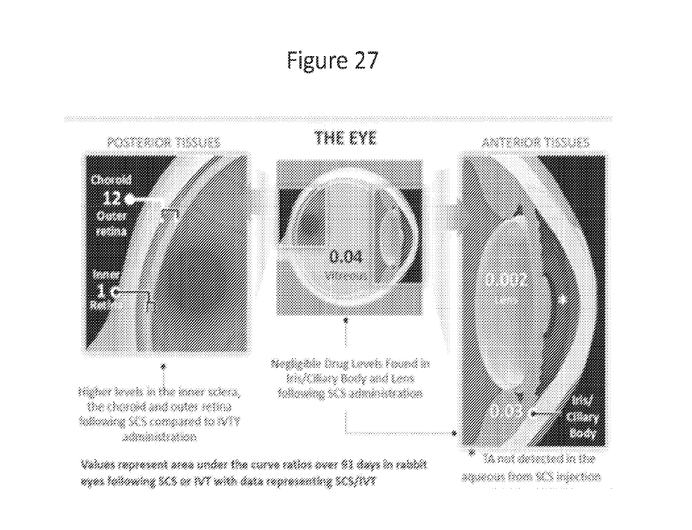

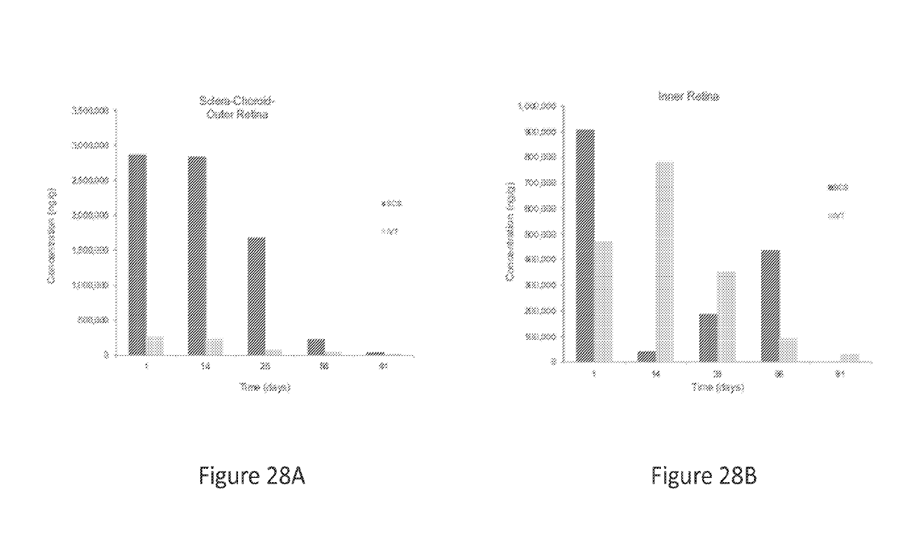

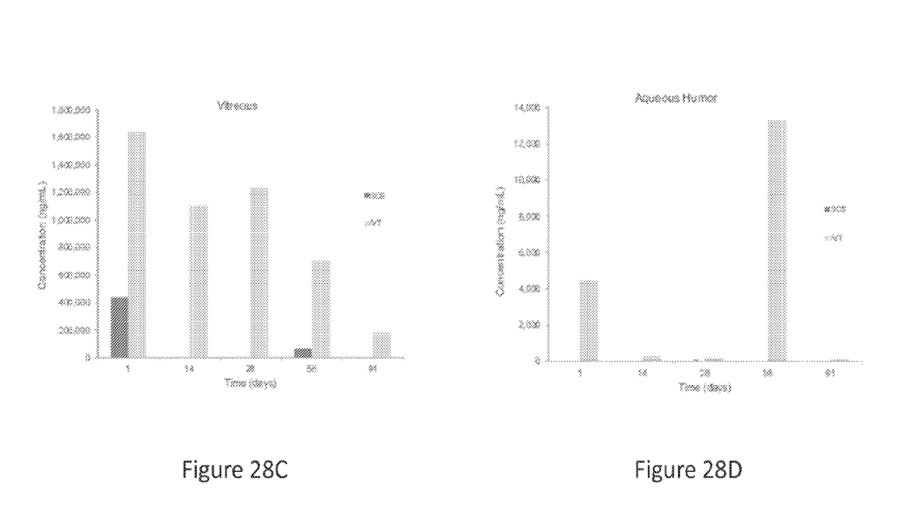

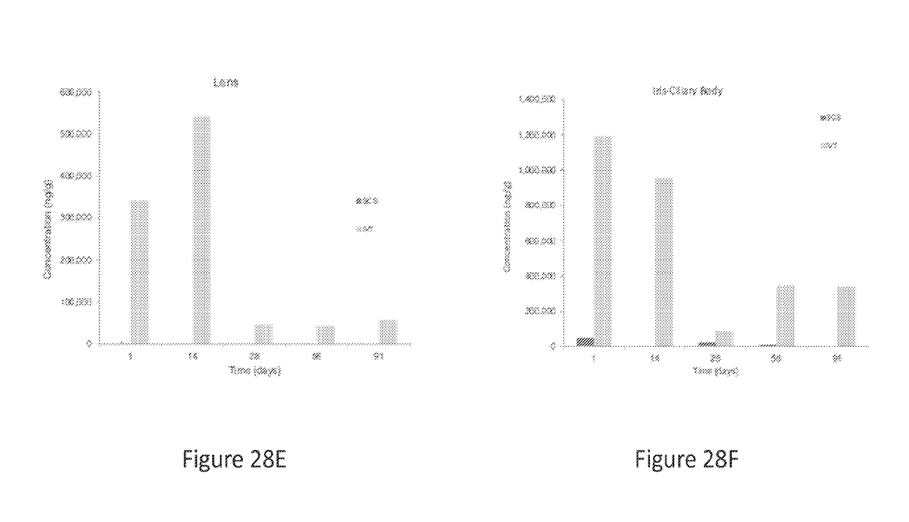

FIGS. 27, 28A-28F illustrate the results of this study. The values shown in FIG. 28 for various parts of the eye represent ratios of total drug over the 91-day time frame of the study, when comparing the two routes of injection. The measures in FIGS. 28A-28F represent the amount of drug found in a specific tissue or area following SCS injection expressed as a proportion of the amount found in the same tissue or area following intravitreal injection. For example, a ratio of 1.0 indicates that there is an equal amount of drug found in the specific tissue following both routes of injection, whereas a ratio of 10 indicates that there is ten times more drug present in the tissue following SCS administration as compared to intravitreal administration. A ratio of 0.03 indicates that there is approximately thirty three times more drug in the specific area following intravitreal injection as compared to SCS injection.

In the case of intravitreal injection, the highest concentrations of Triesence were present in the iris, ciliary body and lens, all of which are located at the front of the eye, throughout the 91-day period. Throughout the period, significantly lower concentrations of Triesence were present in the choroid and outer retina, and almost no Triesence could be seen in the choroid or outer retina by day 91. By contrast, in the case of SCS injection, significantly higher concentrations of Triesence were present in the choroid and outer retina throughout the 91-day period, with only minimal levels present in the iris, ciliary body and lens. These results suggest that drug administered through the SCS can remain localized away from other parts of the eye and that SCS injection provides significantly better bioavailability in the targeted retinal and choroidal tissue than intravitreal injection.

### Example 6

### Study Comparing the Effects of SCS Administration of CLS-TA with SCS Administration of Triesence in Rabbits

Aspects of the invention are directed to a triamcinolone acetonide formulation (“CLS-TA”) having characteristics as provided in Table 5 below:

TABLE 5
CLS1001, triamcinolone acetonide injectable suspension

| | 40 mg/mL | | | |
|---|---|---|---|---|
| Ingredient | formulation | | 8 mg/mL formulation | |
| Triamcinolone acetonide | 40 | mg/mL | 8 | mg/mL |
| Particle Size | D₅₀: ~2 | μm | D₅₀: ~2 | μm |
| | D₉₉: <10 | μm | D₉₉: <10 | μm |
| Sodium Chloride | 0.55% | w/v | 0.55% | w/v |
| Carboxymethylcellulose | 0.5% | w/v | 0.5% | w/v |
| sodium | | | | |
| Polysorbate 80 | 0.02% | w/v | 0.01% | w/v |
| KCl | 0.075% | w/v | 0.075% | w/v |
| CaCl₂ (dihydrate) | 0.048% | w/v | 0.048% | w/v |
| MgCl₂ (hexahydrate) | 0.030% | w/v | 0.030% | w/v |
| Sodium acetate (trihydrate) | 0.39% | w/v | 0.39% | w/v |
| Sodium citrate (dihydrate) | 0.17% | w/v | 0.17% | w/v |

| | | |
|---|---|---|
| NaOH/HCl | Adjust to pH 6.0-7.5 | Adjust to pH 6.0-7.5 |

A pharmacokinetic study in rabbits was conducted comparing the pharmacokinetic profile of CLS-TA with the profile of Triesence, each administered into the SCS. Pharmacokinetics refers to the process by which a drug is distributed and metabolized in the body, which provides information on drug levels in specific tissues and how these levels change over time. Each rabbit received a single dose of 4.0 mg of either CLS-TA or Triesence administered through the SCS on day 1 of the study. The rabbits were then observed for periods of up to 90 days and the resulting concentration of each of the two TA formulations in various parts of the eye was measured at days 15, 29, 58, 63 and 91.

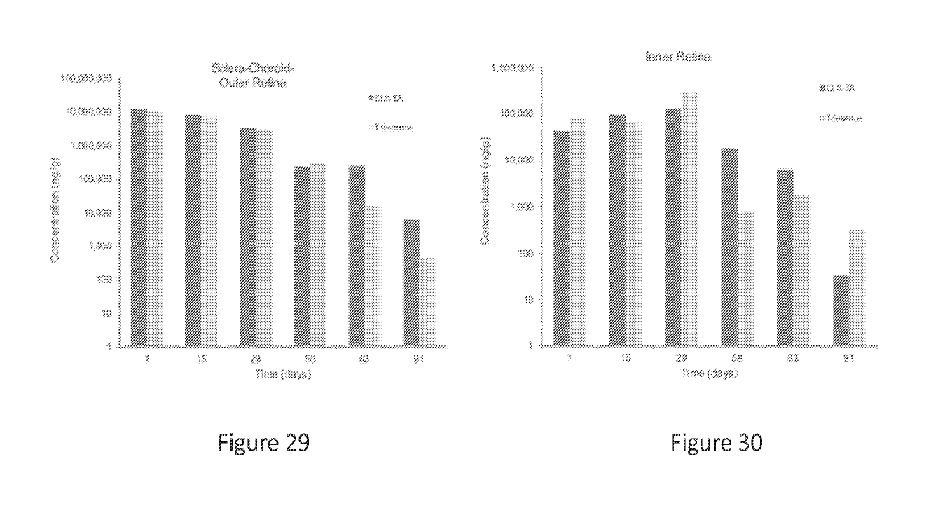

In this study, CLS-TA and Triesence had comparable distributions throughout the eye over the 90-day period. As shown in FIGS. 29-30 both CLS-TA and Triesence, administered into the SCS, remained present in the retina and choroid at high concentration levels throughout the 90-day period following injection.

### Example 7

### Animal Toxicology Studies

Toxicology studies in rabbits demonstrated that both CLS-TA and Triesence were well tolerated when injected to the SCS. In one study, rabbits received a single injection of Triesence into the SCS and were evaluated for the following 17 weeks. In the other study, rabbits received an injection of CLS- TA into the SCS and were evaluated for the following 13 weeks; a subgroup of the rabbits then received a second injection of CLS- TA into the SCS and the rabbits were evaluated for an additional 13 weeks. Both studies showed CLS-TA and Triesence to be generally well-tolerated and safe after single and repeat dosing, supporting the administration of CLS- TA and Triesence in clinical studies.

### Example 8

### Evaluatoion of Suprachoroidal Triamcinolone Injection and Oral Prednisone in a Porcine Model of Uveitis

In this experiment, the anti-inflammatory effects following both high and maintenance daily dose of oral steroid were evaluated, and compared to the anti-inflammatory effects of suprachoroidal steroid injection in a porcine model of acute posterior uveitis. The questions asked included whether administration of triamcinolone to the SCS demonstrated anti-inflammatory properties in the pig model of acute uveitis, whether this effect was comparable to the effect from the most commonly used oral high daily dose regimen in uveitis, and whether the anti-inflammatory effect of triamcinolone matched the oral daily maintenance low dose regimen also used quite often for longer term control of intraocular inflammation. The study design is presented in Table 6 below.

TABLE 6
Study design.

| | | | | Number |
|---|---|---|---|---|
| | | | | of pigs |
| Group | Eyes | Treatment | Exams | per group |
| 1 (negative | OD | LPS/BSS SCS | Time −24 hrs.* | 4 |
| control) | | | (prior to 1^{st} | |
| 2 (oral high | OD | LPS/prednisone | injection of LPS), | 4 |
| dose | | 1 mg/kg/day PO | 0* (prior to | |
| prednisone) | | | treatment), 24, 48 | |
| 3 (CLS-TA) | OD | LPS/2 mg CLS-TA | and 72 hrs. | 4 |
| 4 (oral low | OD | LPS/prednisone | | 4 |
| dose | | 0.1 mg/kg/day PO | | |
| prednisone | | | | |

OD - right eye (Treated) OS - left eye (Untreated)
¹50 ul CLS-TA (Clearside product) or BSS injected into the SCS using microneedles on Day 0, 24 hours after intravitreal LPS, or oral (PO) prednisone on Day 0 and repeated every 24 hours until euthanasia.
²Examinations consist of Modified Hackett McDonald Inflammation Scores and IOPs (TonoVet)
*Examinations at −24 hours (prior to LPS injection) and at day 0 (Prior to treatment), and at 3 days after injection of CLS-TA, consisted of full-field scotopic

Twenty four hours after the induction of acute uveitis by intraocular lipopolysaccharide (LPS) injection (Day 0) into the vitreous, 50 μL of balanced salt solution (BSS, Group 1) or triamcinolone (CLS-TA) (2 mg, Group 3) was injected into the suprachoroidal space (SCS). In Groups 2 and 4, oral prednisone (1 mg/kg/day, Group 2 or 0.1 mg/kg/day, Group 4) was dosed on Day 0, and repeated every 24 hours until euthanasia on day 3. Eyes were examined every 24 hours, which included measuring inflammation scores (Modified Hackett-McDonald) and intraocular pressure (TOP) until euthanasia 3 days after initiation of treatment. Safety assessments and histopathology were performed on all eyes. Electroretinography and wide-field fundus photography was performed at −24 hours, time 0 (before treatment) and on Day 3. Histopathology was performed on eyes after euthanasia.

The oral doses chosen for this study reflected the doses typically used to treat patients with uveitis, for initial dose (1 mg/kg/day) and maintenance dose (0.1 mg/kg/day). Only the right eye of each animal was used in the study and the left eye was unaltered (n=4/group).

Uveitis Model. Twenty-four hours (Time −24) prior to SCS injection of CLS-TA or vehicle, or oral administration of prednisone, and with the pigs anesthetized (intramuscular Telazol-Ketamine-Xylazine and isoflorane in oxygen via mask), 100 ng of lipopolysaccharide (LPS; E. coli 055:B55; Sigma, Inc. St. Louis, Mo.) in 100 uL BSS (Alcon Laboratories, Inc, Forth Worth, Tex.), was injected using a 27 gauge needle into the central posterior vitreous. All injections were performed aseptically. Prior to all ocular injections, the eye was prepped with sterile 5% betadine solution then followed by irrigation with sterile eyewash Immediately following the injections, 1 drop of moxifloxacin ophthalmic solution (Vigamox®, Alcon Laboratories, Fort Worth, Tex.) was applied topically, and the pigs were allowed to recover from anesthesia.

Treatment. Twenty-four hours after the LPS injection (Time 0), 50 uL of CLS-TA (2 mg) (Group 3) or BSS (Group 1) was injected either into the SCS (30 gauge, approximately 1100 μM microneedle) in eyes prepared aseptically. Sterile microneedles were used to inject into the SCS of pigs. All injections were made superiorly (12 o'clock), approximately 5-6 mm posterior to the limbus. In Groups 2 and 4, oral prednisone (Roxane Laboratories, Columbus, Ohio) (1 mg/kg/day PO [Group 2] or 0.1 mg/kg/day PO [Group 4]) was dosed on recovery from anesthesia on Day 0, and repeated every 24 hours until euthanasia.

Ocular Inflammation Score. A modified Hackett-McDonald microscopic ocular inflammation scoring system was used to evaluate the ocular anterior segment, lens, and anterior vitreous. Specifically, both eyes of each animal were examined by a board-certified veterinary ophthalmologist using a handheld slit lamp and indirect ophthalmoscope as follows. Lenticular Examination: Approximately one drop of a short-acting mydriatic solution was instilled onto each eye in order to dilate the pupil. After acceptable dilation has occurred, the lens of each eye was examined using a slit-lamp biomicroscope. Hackett, R. B. and McDonald, T. O. Ophthalmic Toxicology and Assessing Ocular Irritation. Dermatoxicology, 5^{th} edition. Ed. F. N. Marzulli and H. I. Maibach. Washington, D.C.: Hemisphere Publishing Corporation. 1996; 299-305 and 557-566, incorporated by reference herein in its entirety.

Using a portable slit lamp biomicroscope (Zeiss HSO-10, Carl Zeiss Meditec, Inc. USA), ocular inflammation scores were evaluated at Time −24 (prior to LPS injection), at Time 0 (prior to vehicle or CLS-TA injection, then at times 24, 48 and 72 hours after injection. Scores were summed to provide a single inflammation score for each animal for each examination.

Intraocular Pressure. Intraocular pressure (TOP) was measured with the pigs awake and hand restrained at −144, −96, −24, 0, 24, 48, and 72 hours (see FIG. 1) using a TonoVet Tonometer (iCare, Finland). The measurements were performed with the pigs awake and without use of topical anesthetic. The tip of the probe was directed to contact the central cornea and 6 measurements were made consecutively. After the six measurements, the mean IOP was shown on the display providing the IOP that was recorded.

Scotopic Electroretinography (ERG). All animals were dark adapted for 15 minutes prior to ERG. With the pigs anesthetized at times −24, 0 and 72 hours, and pupils dilated with 1% tropicamide HC1, whole field scoptic ERGs were recorded from the right eye prior to injections. A monopolar contact lens electrode (ERG-jet, La Chaux des Fonds, Switzerland) was placed on the cornea to serve as an active electrode. A subdermal electrode at the lateral canthus served as the indifferent electrode. A Barraquer eyelid speculum was placed to maintain open eyelids and a subdermal needle electrode was inserted dorsally as the ground electrode. ERGs were elicited by brief flashes at 0.33 Hz delivered with a mini-ganzfeld photostimulator (Roland Instruments, Wiesbaden, Germany) at maximal intensity. Twenty responses were amplified, filtered, and averaged (Retiport Electrophysiologic Diagnostic Systems, Roland Instruments, Wiesbaden, Germany) B wave amplitudes were recorded from each pig at the designated times.

Wide-field Ocular Fundus Digital Photography. With the pigs anesthetized at times −24, 0 and 72 hours, and pupils dilated with tropicamide 1%, the ocular fundus was photographed with standardized illumination and focus using a wide-field digital imaging system (Retcam II, Clarity Medical Systems, Pleasanton, Calif.).

Ocular Histopathology. The pigs were euthanized at study time 72 hours after clinical scoring, ERG, and wide-field ocular fundus photography was completed. After euthanasia with an overdose of intravenous barbiturate, the right eye was removed. Aqueous humor (AH) was aspirated and the globe was then fixed in Davidson's solution for 24 hours, followed by alcohol. Central, saggital sections of each globe, including the optic nerve, were stained with hematoxylin and eosin and examined by light microscopy. Degree of inflammatory infiltrate of the ocular anterior and posterior segments was graded by two observers masked to the study groups and the final grade was an average of the two scores. The grading scale used was modified from Tilton, et al (IOVS 1994):

Anterior chamber tissues including the iris, ciliary body, ciliary process, corneal endothelium, and the anterior chamber, were scored for severity of inflammation as follows:

0=normal tissue

1=dilated iris vessels and thickened iris stroma with exudate, protein, and/or a few scattered inflammatory cells in the anterior chamber

2=infiltration of inflammatory cells into the stroma of the iris and/or ciliary body, with a moderate number of inflammatory cells within the anterior chamber

3=heavy infiltration of inflammatory cells within the iris stroma and ciliary body and a heavy infiltration of inflammatory cells within the anterior chamber

4=heavy exudation of cells in dense protein aggregation in the anterior chamber and inflammatory cell deposits on the corneal endothelium.

The histologic classification system the retina and posterior segment was:

0=normal tissue

1=minimal infiltration of inflammatory cells within the vitreous cavity and/or retina

2=moderate infiltration of inflammatory cells within the vitreous cavity and/or retina.

3=severe infiltration of inflammatory cells within the vitreous cavity and/or retina

Data and Statistical Analysis. Parametric normally distributed data (i.e., IOP, ERG, retinal thickness) were compared by time point for each group using 1-way ANOVA models with Tukey-Kramer post-hoc analysis. For non-parametric data (i.e., clinical scores, histologic grades), Wilcoxon tests were conducted per animal by time point. Differences were considered significant at P<0.05. Results and probabilities were calculated using computerized statistical software (JMP 10, SAS Inc. Cary, N.C.).

### Results

Injection Procedure Observations. Injections of CLS-TA or BSS into the SCS (Groups 1 and 3) were accomplished using microneedles without difficulty or adverse effect. Eyes were examined via slit lamp biomicroscopy and indirect ophthalmoscopy following each injection. No evidence of backleakage of treatment materials through the microneedle scleral perforation or leakage of drug into the vitreous was observed. Furthermore, there was no evidence of injection site or vitreal hemorrhage following any injections (SCS).

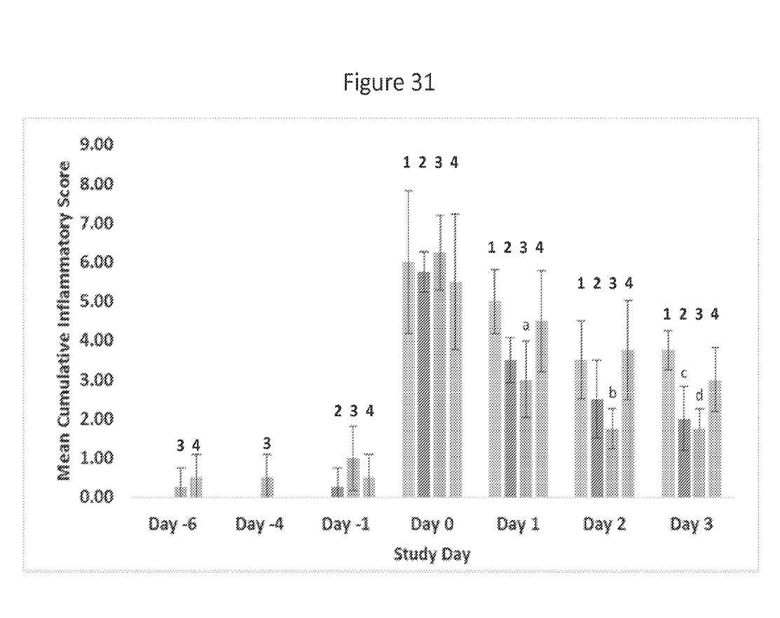

Ocular Inflammation Scores. Mean cumulative inflammation scores as assessed by ophthalmoscopy at time −24 hours ranged between 0 and 1 for all groups and were not significantly different. Following intravitreal injection of LPS, by time 0, mean cumulative inflammation scores elevated to between 5.5 and 6.25 in all groups (FIG. 31) and there were no significant differences between treatment groups. Following treatment, mean inflammation scores generally decreased in all groups over the next 3 days. On Days 1 and 2 (24 and 48 hours after initiation of treatment, respectively), only Group 3 (CLS-TA) had mean cumulative inflammation scores significantly lower than Group 1 (BSS treated; P=0.04 and P=0.023 for Days 1 and 2, respectively). After 72 hours of treatment, Groups 2 (high dose of prednisone) and Group 3 (CLS-TA) had significantly lower mean cumulative inflammation scores than Group 1 (P<0.034). Group 4 (low dose oral prednisone) mean cumulative inflammation scores were not significantly different than saline treated eyes at any treatment time. These results suggest that 2 mg of CLS-TA injected into the SCS resulted in more rapid reduction of inflammation than high dose oral prednisone (one day vs. three days) and both CLS-TA and high dose prednisone were more effective than low dose prednisone in reduction of ocular inflammation in this model of uveitis.

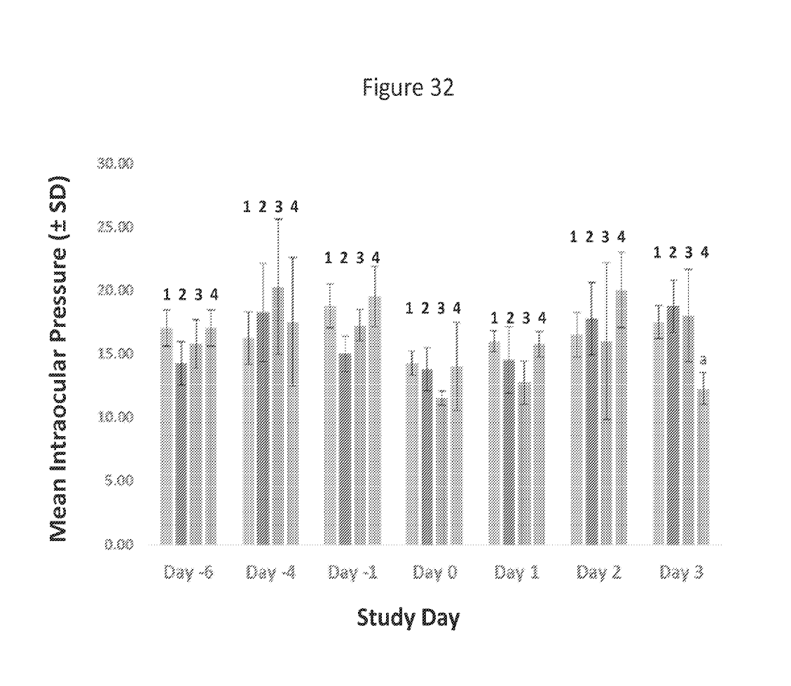

Intraocular Pressure. Mean intraocular pressure ranged from 14.24 to 17 mmHg during acclimation and increased slightly as pigs became accustomed to being handled. On induction of uveitis, the mean IOP decreased by Time 0 to between 11.5 and 14.25 mmHg in all groups, which were not significantly different. Following treatment, IOP returned to baseline by Day 1 in all groups. On Day 3, Group 4 eyes (low dose oral prednisone) had significantly lower IOP than all other groups (P<0.0065) suggesting that these eyes had more inflammation compared to the other groups. IOP in Group 3 eyes stayed substantially constant throughout the study period (FIG. 32).

Electroretinography. At time −24 hours, mean scotopic B wave amplitudes were not significantly different between groups and ranged from 121.9+/−58.7 uV to 220+/−16.04 uV. There were also no significant differences between groups in mean scotopic B wave amplitudes at time 0 (after induction of uveitis) with a range of 92.2+/−15.3 uV to 204 +/−62.0 uV. By Day 3 of treatment, a range of 262.7+/−26.5 uV and 91.2 uV+/−24.5 uV was measured. On Day 3, mean scotopic B wave amplitude in Group 3 (CLS-TA) was significantly lower than other groups (P=0.034). This decreased B-wave amplitude was interpreted as biologic variability and not toxicologically significant as there was no correlating abnormality observed on ocular fundus examination or retinal histology.

Wide-field Ocular Fundus Digital Photography. Wide-field ocular fundus images revealed substantial cloudiness of the ocular posterior segment 24 hours after LPS injection. The cloudiness observed in eyes on Day 0 was a result of predominantly cellular infiltrate into the vitreous humor and some changes to the retina. In BSS treated eyes (Group 1), the cloudiness appeared to worsen from 24 to 72 hours. Treatment with high dose prednisone (Group 2) and CLS-TA (Group 3) resulted in fundus images near pre-treatment appearance at 72 hours. However, treatment with low dose prednisone (Group 4) resulted in images only slightly improved over vehicle treated eyes.

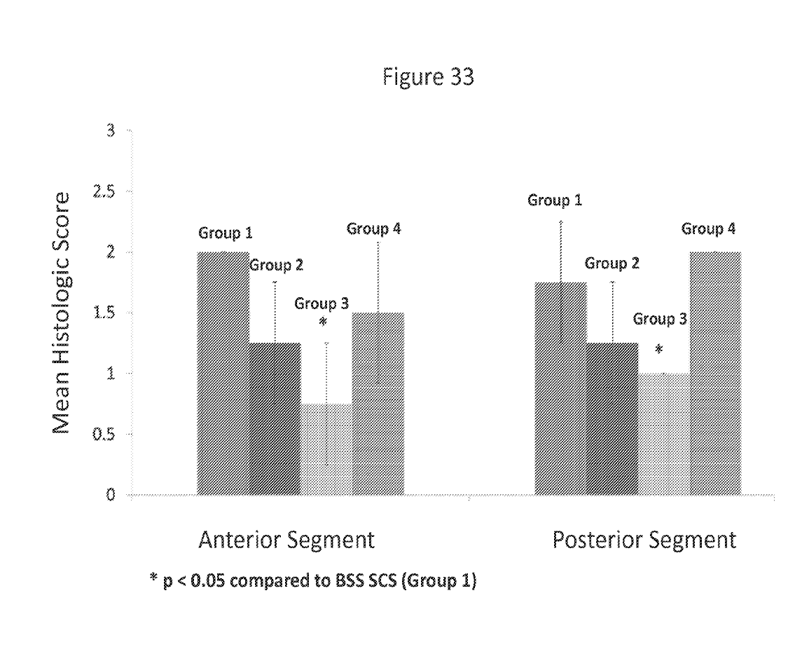

Ocular histopathology. No signs of inflammation or degeneration associated with the SCS injection in Group 3 animals were observed by ocular histopathology. Each eye of this group had evidence of TA crystals in the SCS. There was no evidence of test-article related toxicity of the ocular anterior or posterior segment in any group. Mean histologic scores of the anterior segment of eyes with CLS-TA (Group 3) were significantly lower (P=0.018) than eyes treated with saline (Group 1), while mean anterior segment scores of the eyes treated with oral prednisone (Groups 2 and 4) were not significantly different than Group 1 (FIG. 33). Mean histologic scores of the posterior segment of eyes treated with CLS-TA were significantly lower than eyes treated with saline (Group 1) and eyes treated with high dose prednisone (Group 2) and CLS-TA (Group 3) were lower than the mean score of eyes treated with low dose oral prednisone (Group 4) (P<0.013) (FIG. 33). These results suggest that CLS-TA was as effective as high dose and more effective than low dose oral prednisone in reduction of histologic inflammation compared to saline treated eyes.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. A method of treating macular edema associated with uveitis or macular edema associated with retinal vein occlusion (RVO) in a human subject in need thereof, the method comprising,
in a dosing session, non-surgically administering an effective amount of a drug formulation comprising a first drug to the suprachoroidal space (SCS) of the eye of the human subject in need of treatment of the macular edema associated with uveitis.

2. The method of claim 1, wherein the uveitis is selected from the group consisting of infectious uveitis, noninfectious uveitis, acute uveitis, chronic uveitis, intermediate uveitis, posterior uveitis, and pan uveitis.

3-9. (canceled)

10. The method of claim 1, wherein the RVO is selected from the group consisting of branch retinal vein occlusion (BRVO), hemiretinal vein occlusion (HRVO), and central retinal vein occlusion (CRVO).

11-12. (canceled)

13. The method of claim 1, wherein the effective amount of the drug formulation is present in a volume of from about 10 μL to about 200 μL.

14. (canceled)

15. The method of claim 1, wherein the first drug comprises an anti-inflammatory drug, a non-steroid anti-inflammatory drug (NSAID), or a steroid.

16. The method of claim 15, wherein the anti-inflammatory drug is selected from mycophenolate, infliximab, nepafenac, azathioprine, cyclosphosphamide, dexamethasone, difluprednate, fluocinolone, fluorometholone, leteprednol, prednisolone acetate, prednisolone sodium phosphate, rimexolone, triamcinolone, bromfenac, diclofenac, fluibiprofen, ketorolac, adalimumab, etanercept, certolizumab, gotimumab, daclizumab, rituximab, abatacept, basiliximab, belimumab, anakinra, efalizuma, alefacept, and natalizumab.

17. (canceled)

18. The method of claim 15, wherein the anti-inflammatory drug is triamcinolone acetonide.

19-20. (canceled)

21. The method of claim 1, wherein the intraocular pressure of the eye of the human subject remains substantially constant about 10 minutes, about 20 minutes, about 30 minutes or about 1 hr. after a dosing session of the drug formulation has been completed.

22. The method of claim 21, wherein the intraocular pressure of the eye of the human subject varies by no more than about 10% about 10 minutes, about 20 minutes, about 30 minutes or about 1 hr. after a dosing session of the drug formulation has been completed.

23. The method of claim 1, wherein administration of the first drug to the SCS of the eye provides a decreased number of side effects, or a reduced severity of one or more side effects, as compared to the identical dosage of the first drug administered intravitreally, intracamerally, sub-tenonally, topically, parenterally or orally.

24. The method of claim 1, wherein the dosage of the first drug sufficient to elicit a therapeutic response when administered to the SCS is less than the dosage of the drug sufficient to elicit a therapeutic response when administered intravitreally, intracamerally, sub-tenonally, topically, parenterally or orally.

25-36. (canceled)

37. The method of claim 1, further comprising non-surgically administering a second drug to the eye of the patient.

38. The method of claim 37, wherein the second drug is present in the drug formulation.

39. The method of claim 37, wherein the second drug is present in a second drug formulation.

40. The method of claim 37, wherein the second drug is a VEGF modulator.

41. (canceled)

42. The method of claim 37, wherein the second drug is a VEGF antagonist selected from a VEGF-receptor kinase antagonist, an anti-VEGF antibody or fragment thereof, an anti-VEGF receptor antibody, an anti-VEGF aptamer, a small molecule VEGF antagonist, a thiazolidinedione, a quinoline or a designed ankyrin repeat protein (DARPin).

43. The method of claim 42, wherein the VEGF antagonist is aflibercept, ziv-aflibercept, bevacizumab, sonepcizumab, VEGF sticky trap, cabozantinib, foretinib, vandetanib, nintedanib, regorafenib, cediranib, ranibizumab, lapatinib, sunitinib, sorafenib, plitidepsin, regorafenib, verteporfin, bucillamine, axitinib, pazopanib, fluocinolone acetonide, nintedanib, AL8326, 2C3 antibody, AT001 antibody, XtendVEGF antibody, HuMax-VEGF antibody, R3 antibody, AT001/r84 antibody, HyBEV, ANG3070, APX003 antibody, APX004 antibody, ponatinib, BDM-E, VGX100 antibody, VGX200, VGX300, COSMIX, DLX903/1008 antibody, ENMD2076, INDUS815C, R84 antibody, KD019, NM3, MGCD265, MG516, MP0260, NT503, anti-DLL4/VEGF bispecific antibody, PAN90806, Palomid 529, BD0801 antibody, XV615, lucitanib, motesanib diphosphate, AAV2-sFLT01, soluble Flt1 receptor, AV-951, Volasertib, CEP11981, KH903, lenvatinib, lenvatinib mesylate, terameprocol, PF00337210, PRS050, SP01, carboxyamidotriazole orotate, hydroxychloroquine, linifanib, ALG1001, AGN150998, MP0112, AMG386, ponatinib, PD173074, AVA101, BMS690514, KH902, golvatinib (E7050), dovitinib, dovitinib lactate (TKI258, CHIR258), ORA101, ORA102, Axitinib (Inlyta, AG013736), PTC299, pegaptanib sodium, troponin, EG3306, vatalanib, Bmab100, GSK2136773, Anti-VEGFR Alterase, Avila, CEP7055, CLT009, ESBA903, GW654652, HMPL010, GEM220, HYB676, JNJ17029259, TAK593, Nova21012, Nova21013, CP564959, smart Anti-VEGF antibody, AG028262, AG13958, CVX241, SU14813, PRS055, PG501, PG545, PTI101, TG100948, ICS283, XL647, enzastaurin hydrochloride, BC194, COT601M06.1, COT604M06.2, MabionVEGF, Apatinib, RAF265 (CHIR-265), Motesanib Diphosphate (AMG-706), Lenvatinib (E7080), TSU-68 (SU6668, Orantinib), Brivanib (BMS-540215), MGCD-265, AEE788 (NVP-AEE788), ENMD-2076, OSI-930, CYC116, Ki8751, Telatinib, KRN 633, SAR131675, Dovitinib (TKI-258) Dilactic Acid, Apatinib, BMS-794833, Brivanib Alaninate (BMS-582664), Golvatinib (E7050), Semaxanib (SU5416), ZM 323881 HC1, Cabozantinib malate (XL184), ZM 306416, AL3818, AL8326, 2C3 antibody, AT001 antibody, HyBEV, bevacizumab (Avastin®), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, sunitinib malate (Sutent®), INDUS815C, R84 antibody, KDO19, NM3, allogenic mesenchymal precursor cells combined with an anti-VEGF antagonist (e.g., anti-VEGF antibody), MGCD265, MG516, VEGF-Receptor kinase inhibitor, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Flt1 receptor, cediranib (Recentin™), AV-951, tivozanib (KRN-951), regorafenib (Stivarga®), volasertib (BI6727), CEP11981, KH903, lenvatinib (E7080), lenvatinib mesylate, terameprocol (EM1421), ranibizumab (Lucentis®), pazopanib hydrochloride (Votrient™), PF00337210, PRS050, SP01 (curcumin), carboxyamidotriazole orotate, hydroxychloroquine, linifanib (ABT869, RG3635), fluocinolone acetonide (Iluvien®), ALG1001, AGN150998, DARPin MP0112, AMG386, ponatinib (AP24534), AVA101, nintedanib (VargatefrM), BMS690514, KH902, golvatinib (E7050), everolimus (Afinitor®), dovitinib lactate (TKI258, CHIR258), ORA101, ORA102, axitinib (Inlyta®, AG013736), plitidepsin (Aplidin®), PTC299, aflibercept (Zaltrap®, Eylea®), pegaptanib sodium (Macugen™, LI900015), verteporfin (Visudyne®), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPL010, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PTI101, TG100948, ICS283, XL647, enzastaurin hydrochloride (LY317615), BC194, quinolines, COT601M06.1, COT604M06.2, MabionVEGF, SIR-Spheres coupled to anti-VEGF or VEGF-R antibody, Apatinib (YN968D1), or AL3818.

44-46. (canceled)

47. The method of claim 37, wherein the second drug is administered to the suprachoroidal space (SCS) of the eye of the subject.

48. The method of claim 37, wherein the second drug is administered intravitreally in a second drug formulation.

49-53. (canceled)

54. The method of claim 1, wherein subsequent to a dosing session, the patient substantially maintains his or her vision, as measured by losing fewer than 15 letters in a best-corrected visual acuity (BCVA) measurement, compared to the patient's BCVA measurement prior to the dosing session, and the loss of fewer than 15 letters is measured at least about 1 week, at least about 2 weeks, at least about 1 month, at least about 2 months, at least about 3 months or at least about 4 months after the at least one dosing session.

55. The method of claim 1, wherein the patient experiences an improvement in vision subsequent to a dosing session, as measured by gaining ≧5 letters, ≧10 letters or ≧15 letters in a best-corrected visual acuity (BCVA) measurement, compared to the patient's BCVA prior to the dosing session, and the gain of letters in the BCVA is measured at least about 1 week, at least about 2 weeks, at least about 1 month, at least about 2 months, at least about 3 months or at least about 4 months subsequent to the at least one dosing session.

56. (canceled)

57. The method of claim lany onc of claims 1, wherein subsequent to the at least one dosing session in the eye in need of treatment, the patient experiences a decrease in retinal thickness in the treated eye, as measured by optical coherence tomography (OCT) compared to the patient's retinal thickness in the eye in need of treatment prior to the at least one dosing session, and the decrease in retinal thickness is measured at least about 1 week, at least about 2 weeks, at least about 1 month, at least about 2 months, at least about 3 months or at least about 4 months after the at least one dosing session.

58. The method of claim 57, wherein the retinal thickness is central subfield thickness (CST).

59. The method of claim 57, wherein the decrease in retinal thickness is ≧25 μm, ≧50 μm, ≧75 μm or ≧100.

60. The method of claim 57, wherein the decrease in retinal thickness is ≧5%, ≧10% or ≧25%.

61-102. (canceled)

103. The method of claim 1, wherein upon administration, the drug formulation flows away from the insertion site and is substantially localized to the posterior segment of the eye.
